SEQUENCE LISTING (4 sequences)

SEQ ID NO: 1 - DNA, 20 bp, synthetic construct
  source 1..20: mol_type = other DNA
accgtggaaggttcttctgg

SEQ ID NO: 2 - DNA, 22 bp, synthetic construct
  source 1..22: mol_type = other DNA
cacaaagagtaggacttgaccc

SEQ ID NO: 3 - DNA, 20 bp, synthetic construct
  source 1..20: mol_type = other DNA
tgtgtccgtcgtggatctga

SEQ ID NO: 4 - DNA, 21 bp, synthetic construct
  source 1..21: mol_type = other DNA
ttgctgttgaagtcgcaggag

(19) United States
(12) Patent Application Publication
(10) Pub. No.: US 2024/0093289 A1
(43) Pub. Date: Mar. 21, 2024

(54) Image-seq: A New Technology for Spatially-Resolved Single-Cell RNA Sequencing

(71) Applicant: The General Hospital Corporation, Boston, MA (US)

(72) Inventors: Christa Haase, Somerville, MA (US); Dmitry Richter, Somerville, MA (US); Charles Lin, Arlington, MA (US)

(21) Appl. No.: 18/363,298

(22) Filed: Aug. 1, 2023

Related U.S. Application Data
(60) Provisional application No. 63/394,274, filed on Aug. 1, 2022.

(51) Int. Cl. C12Q 1/6869; A61B 10/02; A61B 18/20; A61B 90/20

(52) U.S. Cl. CPC C12Q 1/6869; A61B 10/02; A61B 18/20; A61B 90/20; A61B 2018/00565

(57) ABSTRACT

A system and method for image-guided cell isolation from a region of interest of a subject is disclosed. The system and method include imaging the subject using optical microscopy to identify the region of interest in a target anatomy, inserting a micropipette into the region of interest under guidance of the optical microscopy, and aspirating at least one cell of a target population of cells in the region of interest under guidance of the optical microscopy. The method further includes analyzing the at least one cell aspirated from the region of interest.

### CROSS REFERENCE TO RELATED APPLICATIONS

The present application is based on, and claims priority to, U.S. Provisional Application Ser. No. 63/394,274, filed Aug. 1, 2022, which is incorporated herein by reference in its entirety for all purposes.

### STATEMENT OF GOVERNMENT SUPPORT

This invention was made with government support under 5R01CA194596-04, 5R01DK115577-05, P01HL142494-04, and 5R01DK123216-03 awarded by the National Institutes of Health (NIH). The government has certain rights in the invention.

### INCORPORATION BY REFERENCE OF SEQUENCE LISTING

The present application is being filed with a Sequence Listing in electronic format. The Sequence Listing is provided in XML format as a file entitled “2023-10-7_125141.04359_WIPO_Sequence_Listing_XML.xml”, which is 4,492 bytes in size and was created on Oct. 7, 2023. The sequence listing is electronically submitted with this application via Patent Center and is incorporated herein by reference in its entirety.

### BACKGROUND

Spatial transcriptomics is a rapidly advancing field, encompassing a range of different technologies and capable of spatially-resolved gene expression analysis. In contrast to single-cell RNA sequencing, which provides deep insights into heterogeneities within cell populations but does not preserve the spatial relationship among individual cells, techniques such as MERFISH, seqFISH, and Slide-seq can link these heterogeneities to differences in spatial composition and cellular proximity. However, apart from Niche-seq, they have either been applied only in vitro, or rely on generating tissue sections, which has confined their applicability to tissues that are easily sectioned. They map the gene expression profiles onto two-dimensional images, and extrapolation to three-dimensional architecture of intact tissues is still limited. For example, 3D expression profiles and cell segmentation have been demonstrated in tissue sections but the section thickness is limited by mRNA probe diffusion. In addition, tissue sections can provide only static images, necessitating the use of indirect methods such as pseudo-time analysis to infer cellular trajectories over time, and none of the currently available spatial transcriptomics and multi-omics technologies have been combined with in vivo imaging.

### SUMMARY

The present disclosure provides systems and methods that overcome one or more of the aforementioned drawbacks via methods and systems that enable image-guided cell isolation for scRNA-seq and multi-omics analysis, which may be referred to as Image-seq herein. The core of the Image-seq system may include a one-photon or multiphoton microscope with two optical paths, one for imaging and one for laser micromachining, or a single optical path for both imaging and micromachining which creates an access channel in tissue through which a micropipette is brought to the target location and aspirates cells under image guidance. Because it captures viable cells, Image-seq can be combined with state-of-the-art library preparation protocols, leading to higher mRNA detection efficiencies and broader transcript coverage than other spatial sequencing technologies. In addition, standard computational tools can be used for data analysis.

According to one aspect of the present disclosure, a method for image-guided cell isolation from a region of interest of a subject is disclosed. The method includes imaging the subject using optical microscopy to identify the region of interest in a target anatomy, inserting a micropipette into the region of interest under guidance of the optical microscopy, and aspirating at least one cell of a target population of cells in the region of interest under guidance of the optical microscopy. The method further includes analyzing the at least one cell aspirated from the region of interest.

According to another aspect of the present disclosure, a method for image-guided tissue ablation from a region of interest of a subject is provided. The method includes the steps of imaging the subject using optical microscopy to identify a tissue in a region of interest of a target anatomy and ablating the tissue in the region of interest using an ablation laser under guidance of the optical microscopy. The method further includes confirming a spatial location of the ablated tissue using the optical microscopy.

According to another aspect of the present disclosure, a system for image-guided cell isolation from a region of interest of a subject is provided. The system includes an optical microscope, a flushing assembly, a micropipette assembly, and a processor in communication with the optical microscope and the micropipette. The processor is configured to image the subject using the optical microscope to identify the region of interest in a target anatomy, identify from one or more images generated of the subject at least one cell of the target population of cells in the region of interest under guidance of the optical microscope, and control the micropipette assembly to aspirate at least one cell of a target population of cells in the region of interest under guidance of the optical microscope.

These aspects are nonlimiting. Other aspects and features of the systems and methods described herein will be provided below.

### BRIEF DESCRIPTION OF THE DRAWINGS

The foregoing features of embodiments will be more readily understood by reference to the following detailed description, taken with reference to the accompanying drawings, in which:

FIG. 1A is a schematic of a cell aspiration system, according to aspects of the present disclosure.

FIG. 1B is a schematic of a cell aspiration and tissue ablation system, according to aspects of the present disclosure.

FIG. 2A is flow chart of a method for cell aspiration, according to aspects of the present disclosure.

FIG. 2B is a flow chart of a method for tissue ablation, according to aspects of the present disclosure.

FIG. 3A is an example of a detailed design for the optical microscopy with two optical paths, one for imaging and one for laser micromachining, according to aspects of the present disclosure.

FIG. 3B is an example of a detailed design for optical microscopy with a single optical path for both imaging and micromachining according to aspects of the present disclosure. Radiation at 775 nm is generated by frequency doubling the 1550 nm output from a fiber laser. Radiation at 920-980 nm is generated by soliton self-frequency shift of 1550 nm radiation to 1840-1960 nm in a LMA photonic crystal fiber, which is subsequently frequency doubled to obtain the imaging wavelength. A scanning GM (y scan) and scanning PM (x scan) are conjugated to one another, along with the back aperture of the OL so that a scan along the x and y dimensions is achieved in the image plane. Multiphoton emission wavelengths are collected with a combination of DCs and PMTs to generate an image. A confocal reflectance channel that utilizes an APD and 20 μm-size pinhole can also be used to generate images. λ/2: λ/2-plate. PBS: polarization beam splitter. BiBO: BiBO3—Bismuth orthoborate crystal. LMA: large mode-area. APD: avalanche photodiode. ph: pinhole. DC: dichroic mirror. GM: galvo mirror. PM: polygonal mirror. PMT: photomultiplier tube. MP: micropipette. OL: objective lens.

FIG. 4 is a simplified example optical diagram of the optical design of FIG. 3 showing the integral components for combined multiphoton microscopy and laser ablation setup. CP: conjugate plane, L: lens, M: mirror.

FIG. 5A is a schematic of an example optical setup for cell aspiration, according to aspects of the present disclosure. The setup shows multiphoton microscopy of a subject.

FIG. 5B is a diagram of an example optical setup for cell aspiration, according to aspects of the present disclosure. The setup shows the insertion of a micropipette into the bone marrow for cell aspiration.

FIG. 5C is a diagram of an example optical setup for tissue ablation, according to aspects of the present disclosure. The setup shows plasma-mediated laser ablation.

FIG. 5D is a diagram of an example cell aspiration procedure with laser-assisted tissue microdissection under guidance from an optical microscope, according to aspects of the present disclosure.

FIG. 6A is a schematic of the micropipette assembly and micropipette positioned next to a target cell for aspiration, according to aspects of the present disclosure.

FIG. 6B shows chronological optical images of cell aspiration with the micropipette (dashed line outline) from the region of interest in vivo, transferred into the micropipette, and extracted, according to aspects of the present disclosure.

FIG. 7A is characterization results of micropipette samples. Tiled, maximum intensity projection image of a 4×8 mm region of skull bone marrow (tiles shown as squares on the schematic of the skull below the image), obtained by stitching together individual microscope fields of view. Green, β-actin-GFP-positive bone marrow cells; gray, second-harmonic generation (SHG) bone signal. White square: region of interest that is shown in the insets before and after cell isolation. Scale bars: main image, 1 mm; insets, 100 Tiled calvarium bone marrow images were obtained from three independent mice.

FIG. 7B is characterization results of micropipette samples. Average cell viability for the calvarium (MC), from a total of nine micropipette samples n=3 mice. Cell viability for the tibia (MT), from four micropipette samples in n=2 mice.

FIG. 7C is characterization results of micropipette samples. 3D visualization of a target location before and after cell aspiration. The bottom panel is obtained by rotating the top panel by 180° around the x axis. Isolated cell volume is marked by an orange outline, and the extracted cell volume (diameter ˜80 μm, height 50 μm) contained in a typical micropipette sample is outlined by a cylinder.

FIG. 7D is characterization results of micropipette samples. Averaged cell proportions from flow cytometry analysis of 15 calvarial micropipette (M) and 8 whole calvarial bone marrow (W) preparations in a total of n=10 mice, and 14 tibia micropipette (M) and 5 whole tibia bone marrow (W) preparations in a total of n=7 mice. Cells were classified based on their cell surface properties as T cells: Ter119−CD71−, CD11b−, CD3+; mature B cells: Ter119−CD71−, CD3−, CD11b−, B220+, CD19+, cKit− IgM+; pro-B cells: Ter119−CD71−, CD3−, CD11b−, B220+, CD19+, IgM−, cKit+; pre-B cells: Ter119−CD71−, CD3−, CD11b−, B220+, CD19+, IgM−, cKit−; Pre-Pro-B cells: Ter119−CD71−, CD3−, CD11b−, CD19−, B220+; macrophages: Ter119−CD71−, CD3−, CD11b+, Ly6C−, F4/80+; inflammatory monocytes: Ter119−CD71−, CD3−, CD11b+, F4/80−, Ly6CHigh; patrolling monocytes: Ter119−CD71−, CD3−, CD11b+, F4/80−, Gr1−, Ly6CLow; granulocytes: Ter119−CD71−, CD3−, CD11b+, F4/80−, Ly6CLow, Gr1+.

FIG. 7E is characterization results of micropipette samples. Maximum intensity projection image of a bone marrow region before and after stromal cell aspiration with the micropipette. Red, CXCL12-DsRed bone marrow cells; gray, SHG bone signal. Scale bar, 100 μm. Fifty biological replicates of stromal cell extraction were performed in 14 independent mice.

FIG. 8A is the gating strategy for the analysis of various leukocyte populations in a whole calvarium bone marrow preparation (WCBM) and concatenated data from all micropipette samples. Analyzed cells were gated on singlet live cells and percentages refer to percent of parent gate. The same gating strategy was used for the analysis of whole tibia and tibia micropipette samples.

FIG. 8B is the gating strategy for flow cytometry analysis of CXC112⁺, CD45⁻ stromal cells from 5 concatenated micropipette samples.

FIG. 9A is a 3D visualization of a target location in the tibia of a β-actin-GFP mouse before and after cell aspiration. The bottom panel is obtained by rotating the top panel by 180° around the x axis. The isolated cell volume is marked by an orange outline.

FIG. 9B is a comparison of micropipette and whole bone marrow preparations from the calvarium and tibia. Statistical analysis was performed using the two-way ANOVA for each individual cell population, with the two independent variables being bone type (tibia vs calvarium) and procedure (micropipette vs whole bone marrow preparations). Listed p values were not adjusted for multiple comparisons. Using a Bonferroni correction, statistical significance is achieved for p<0.006 (α=0.05).

FIG. 10A shows the scRNA-seq workflow. For the experiments in FIGS. 10B-12, step 5a was chosen as the final step. Scale bar, 50 μm.

FIG. 10B shows the UMAP embedding of joint alignment of all Image-seq data, color-coded by the major cell populations. B, B cell; DC, dendritic cell; DP, diverse progenitors; GP, granulocyte progenitor; MP, monocyte progenitor; Pre/Pro, Pre- and Pro-B cell; Mono, monocyte. UMAP: uniform manifold approximation and projection.

FIG. 10C shows the UMAP embedding of joint alignment of all WCBM data, color-coded by the major cell populations. UMAP: uniform manifold approximation and projection.

FIG. 10D is a heatmap showing Spearman correlation coefficients of gene average expression level between WCBM and Image-seq for each cell type.

FIG. 10E shows dot plots showing selected marker gene expression across major cell populations for the combined Image-seq and WCBM data. The color represents scaled average expression of marker genes in each cell type, and the dot size indicates the proportion of cells expressing the individual marker gene.

FIG. 10F shows violin plots comparing the number of expressed genes and total unique molecular identifiers (UMIs) per cell between WCBM and Image-seq samples. Statistical significance was determined using a two-sided Wilcoxon rank sum test.

FIG. 10G is a tiled, maximum intensity projection image of leukemic bone marrow at day 10 after transplantation; examples of high-burden (HB) and low-burden (LB) regions marked by white squares and shown in the inset. Green, AML-GFP; gray, bone SHG. Scale bars: main image, 1 mm; insets, 50 μm. Also included is a distribution of GFP expression in UMAP embeddings of scRNA-seq data obtained from HB and LB regions. Tilescans were obtained from n=5 independent mice. A total of n=4 biological replicates of cell extraction from HB and n=4 biological replicates of cell extraction from LB regions were performed in six independent mice. A total of four HB and LB cell extractions were performed in the same mice from which we also obtained tilescans. Sequencing was performed on one LB and one HB sample.

FIG. 10H shows UMAP embeddings of HB and LB samples color coded by cell type, and using the annotations given in FIG. 10B. UMAP: uniform manifold approximation and projection.

FIG. 11A shows the distribution of cell proportions for the major hematopoietic cell populations from FIGS. 10B-10C, shown as averaged proportions over all Image-seq vs all WCBM samples.

FIG. 11B shows the breakdown of FIG. 11A showing the distribution of cell proportions for each sample. WCBM samples outlined by red border, Image-seq samples outlined by blue border.

FIG. 11C shows a table with p values for differences in cell proportions between WCBM and Image-seq samples (obtained using a two-sided Wilcoxon rank sum test).

FIG. 11D shows the top GO terms obtained from upregulated genes in Image-seq samples, broken down by cell type.

FIG. 11E shows the top GO terms obtained from downregulated genes in Image-seq samples, broken down by cell type. WCBM: whole calvarial bone marrow. GO: gene ontology. Pre/Pro-B cell: Pre and pro B cell. DP: Diverser progenitor.

FIG. 12A shows the percentage of cells that passed quality control after sequencing in each 10× run (WCBM N=3, Image-seq N=11 biological replicates). A two-sided Mann-Whitney test was used to assess statistical significance. 3 (WCBM) and 5 (Image-seq) independent mice were used, respectively.

FIG. 12B shows the number of unique molecular identifiers and expressed genes per cell for each Image-seq and whole calvarium bone marrow (WCBM) sample.

FIG. 12C shows a table that summarizes the number of cells sequenced from each 10× run, along with the number of cells and percentage of cells that passed quality control.

FIG. 12D is a comparison of the number of expressed genes and total number of molecules (=total UMI) between our WCBM, Image-seq and AML SMARTseq-v4 data. Statistical significance was determined using a two-sided Wilcoxon rank sum test.

FIG. 12E are plots of the number of expressed genes per cell and total mapped reads (=molecules) per cell for the AML SMARTseq-v4 data, including scatter plot. WCBM N=5295 (3 independent mice), Image-seq N=8078 (5 independent mice), SMARTseq N=84 cells (11 independent mice). Boxplot represents the interquartile range (IQR, the range between the 25th and 75th percentile), the mid-point of the data is indicated as a line within. Whiskers indicate the upper and lower value that is within 1.5 times the IQR.

FIG. 13A is an example of the gating strategy used to isolate CXC112⁺, CD45⁻ stromal cells from a micropipette sample for cell lysis and SMARTseq-v4 library preparation. Sorted cells were gated on singlet live cells (DAPI−).

FIG. 13B shows UMAP embedding of the stroma SMARTseq-v4 data using the top 30 principal components, color coded by cell population. UMAP: uniform manifold approximation and projection.

FIG. 13C is a heatmap of scaled normalized expression for the 20 most highly expressed genes within each cell cluster.

FIG. 13D is a heatmap of scaled normalized expression for Lepr-MSC and Endothelial cell marker genes from the literature. MSC: Mesenchymal stromal cells, EC: endothelial cells.

FIG. 14A is a schematic of in vivo imaging timepoints. IVM, intravital microscopy.

FIG. 14B is a plot of cell counts per bone marrow cavity on day 0 (n=143 cavities, n=6 independent mice), day 1 (n=103 cavities, n=6 independent mice) and day 3 (n=132 cavities, n=7 independent mice) after transplantation. Error bars represent the standard deviation and are centered at the arithmetic mean. The 99.9% confidence interval for proliferating (P) cells is shown in red, that for intermediate (IM) cells in blue and that for non-proliferating (NP) cells in green.

FIG. 14C is a plot of the number of cells per cavity on day 3 plotted for P, IM, and NP regions. The dashed line represents the median and the dotted line, the quartiles in each distribution. Statistical significance was determined using Welch's ANOVA test and the adjusted P values were determined using Dunnett's multiple comparisons test. NP, 10 biological replicates, n=3 independent mice; IM, 32 biological replicates, n=3 independent mice; P, 107 biological replicates, n=3 independent mice.

FIG. 14D shows tiled, maximum intensity projection image of leukemic bone marrow on day 3 after transplantation. Red square and inset, P cells; blue square and inset, IM cells; green square and inset, NP cell (marked by white arrow). Scale bars: main image, 500 μm; insets, 100 Bone, bone SHG. Imaging of the entire calvarium bone marrow at day 3 was performed in 17 independent mice, including 122 biological replicates with P, 35 biological replicates with IM, and 79 biological replicates with NP cells.

FIG. 14E shows images of proliferating (top; scale bars, 100 μm) and non-proliferating (bottom; scale bars, 50 AML cells imaged longitudinally in the same animal and bone cavity on day 1 and 3 after transplantation. Bone SHG signal is shown in grey, AML-GFP signal is shown in green. The same depth along the z dimension is shown (Zrel=0) on both days, along with a maximum intensity projection for P cells. For the NP cell a single Z slice (Zrel=28 μm) shows that the AML cell has moved deeper into the bone marrow on day 3. A total of 107 biological replicates with P, 10 biological replicates with NP and 32 biological replicates with IM cells (n=3 independent mice, respectively) were identified by longitudinal imaging (definition of P, NP, IM based on the fold-change difference between day 1 and day 3).

FIG. 14F are distributions of the cell cycle in the three imaging phenotypes (P value obtained using a two-sided Fisher's exact test).

FIG. 14G is a bar-plot representing the fraction of P, NP, and IM cells in each AML cluster. AP1, AML-AP1; GMP, AML-GMP; Mono, AML-mono (P value obtained using a two-sided Fisher's exact test).

FIG. 14H shows a plot of Leiden clustering of 84 AML cells isolated by Image-seq after regression of cell cycle genes using Seurat, visualized as UMAP embedding.

FIG. 14I is a heatmap of scaled normalized expression for the 10 most strongly upregulated genes (based on logFoldChange) in each AML cluster.

FIG. 15A is a plot showing the percentage of AML cells that are CellTracker Red+ on day 3 (CellTracker+(%)) as a function of the number of cells per cavity.

FIG. 15B is violin plots of selected cell cycle relevant genes. Statistical significance was determined using a two-sided Student's t-test (Sample size: NP=33, IM=17, P=34 cells, 11 independent mice).

FIG. 15C is a heatmap showing scaled average expression of gene sets in distinct phase of cell cycle, each column indicates a cell. AML cells are clustered into three groups by hierarchical clustering based on cell cycle genes.

FIG. 15D shows representative images of P and NP cells from mice injected with rat IgG2a,κ-AF568 antibody (DPP4 isotype control). Imaging of DPP4 isotype control labeling was performed in 14 biological replicates with NP cells and 6 biological replicates with P cells (1 independent mouse). Definition of P/NP/IM based on cell number at day 3.

FIG. 16A is the gating strategy used for sorting AML cells into individual wells. Examples of GFP gating strategy used to isolate proliferating and non-proliferating HA9MI cells for cell lysis and SMARTseq-v4 library preparation.

FIG. 16B is gating strategy for exclusion of DAPI-labeled (dead) cells.

FIG. 17A is a plot of differentially expressed genes between P and NP cells calculated using DESeq2, which determines statistical significance using the Wald test and uses an interpretation of the Benjamini-Hochberg method to control the false discovery rate. The vertical dashed lines show the cut-off for gene filtering (log₂ FoldChange 2 and −2) and the horizontal dashed line signifies an adjusted P value of 0.01.

FIG. 17B is a plot showing the percentage of leukemia cells expressing DPP4 during disease progression, as analyzed using cell surface staining and flow cytometry. DPP4⁺ cells are defined as DPP4high and DPP4int cells according to the gating strategy disclosed herein (day 4 n=2, day 3, and 4 weeks n=3, days 9-13 and 2 weeks n=3, days 5-7 n=5 biological replicates). Error bars denote standard deviation. Data were collected from three independent experiments.

FIG. 17C is a plot showing DPP4 antibody labeling detected using intravital microscopy after i.v. injection of DPP4-AF568 antibody. Ratio of red (that is, DPP4-AF568) to green (that is, AML-GFP) fluorescence is plotted for P (5 biological replicates) and NP cells (25 biological replicates), as well as control P (6 biological replicates) and NP (14 biological replicates) cells labeled with isotype antibody (IT). Statistical significance was determined using a two-tailed Mann-Whitney test. Data were collected from three independent mice.

FIG. 17D shows examples of intravital multiphoton microscopy images used for the analysis in c. Red (DPP4-AF568), green (HA9M1-GFP), gray (bone SHG signal) channels and merged images are shown at a single z plane. Images of P cells have red borders; images of NP cells have green borders. Scale bars, 50 μm. Imaging of DPP4 antibody staining: 25 biological replicates with NP cells and 5 biological replicates with P cells (two independent mice). For FIGS. 17B and 17D the definition of P/NP/IM is based on cell number at day 3.

FIG. 17E is a plot of cell cycle analysis on day 7 after transplantation for DPP4⁺ and DPP4⁻ AML cells by Ki-67 staining in HA9M1 cells. (HA9M1, n=8 biological replicates).

FIG. 17F is a plot of cell cycle analysis on day 7 after transplantation for DPP4⁺ and DPP4⁻ AML cells by Ki-67 staining in an MLL-AF9 leukemia model. (MLL-AF9, n=3 biological replicates).

FIG. 17G is the overlap of the top 300 DPP4-correlated genes in the Oregon Health and Science University (Tyner, J. W. et al. Functional genomic landscape of acute myeloid leukemia. Nature 562, 526-531 (2018)) and Firehose (Acute myeloid leukemia (TCGA, Firehose Legacy) datasets plotted as a Venn diagram.

FIG. 17H shows the top GO terms for overlapping genes from FIG. 17G. The statistical analysis was done using a hypergeometric test. HA9M1, Hoxa9/Meis1-Ubiquitin-c-GFP.

FIG. 18A shows Ki-67 flow cytometry analysis of HA9M1 cells 13 days after transplantation (N=7 biological replicates, 2 independent experiments). Statistical significance was determined by unpaired, two-sided Student's t-test, error bars denote standard error of the mean.

FIG. 18B shows Ki-67 cell cycle gating strategy.

FIG. 18C is the gating strategy used for identifying DPP4high, DPP4int and DPP4neg cells.

FIG. 18D is a plot of Day 10 leukemia burden in mice transplanted with 1,000 DPP4high (N=3) or DPP4neg (N=4) HA9M1 cells (2 independent experiments) (left). Error bars denote standard deviation. The Percentage of DPP4high-int-neg cells isolated from recipients transplanted with DPP4high (N=3, red bars) or DPP4neg (N=4, beige bars) HA9M1 cells (2 independent experiments) (Right). An unpaired, two-sided Student's t-test was used for statistical analysis, error bars denote standard deviation.

FIG. 18E shows plots of mean fluorescence intensity of Itgb7, Flt3 and CD48 detected in DPP4high-neg HA9M1 cells by cell surface staining and flow cytometry 3 weeks after transplantation (N=5 biological replicates, 2 independent experiments). Statistical significance was determined by one-way ANOVA followed by Tukey's post-hoc test, error bars denote standard deviation.

FIG. 18F DPP4, Itgb7, Flt3 and CD48 expression level for the AML clusters from FIG. 14H. A two-sided Wilcoxon rank sum test was used for statistical analysis (AML-AP1 N=21, AML-GMP N=28, AML-mono N=35 cells, 11 independent mice).

FIG. 18G shows the GO analysis of top 300 up-regulated genes comparing P and NP cells. The statistical analysis was performed by hypergeometric test. Terms related to the inhibition of T cells are highlighted by the red rectangle. HA9M1: Hoxa9/Meis1-Ubiquitin-c-GFP. GO: gene ontology.

FIG. 19A is a plot showing the percentage of DPP4+ cells in various myeloid cell populations (N=6 biological replicates). Error bars denote standard deviation. Data were collected from two independent experiments. LKS, Lineage-cKit+Sca-1+; MEP, megakaryocytic-erythroid progenitor; CMP, common myeloid progenitor; StemGMP, Stem-like granulocyte-monocyte progenitor; MonoGMP, monocytic granulocyte-monocyte progenitor; GrGMP, granulocytic granulocyte-monocyte progenitor; Inf monocytes, inflammatory monocytes; Patr monocytes, patrolling monocytes.

FIG. 19B shows a gating strategy for the myeloid cell populations from FIG. 19A, along with DPP4 and isotype count data for each cell type. Analyzed cells were gated on singlet live cells and percentages refer to percent of parent gate. Gray curves represent the Isotype control, red curves DPP4.

FIG. 20A is a plot of cell surface antibody labeling with DPP4 in HA9M1, analyzed by flow cytometry.

FIG. 20B is a plot of the analysis of Dpp4 expression by QPCR on in vitro cultured HA9M1 and MLL-AF9 cells. Results are compared to splenic T cells, which express reproducible levels of Dpp4. (N=3 biological replicates from 2 independent experiments). Error bars denote standard deviation.

FIG. 20C is a plot of the flow cytometry analysis of DPP4 expression in HA9M1 by intracellular staining with DPP4 antibody.

FIG. 20D is a plot of the analysis of Dpp4 by QPCR on AML cells collected from the bone marrow 3 weeks after transplantation. DPP4⁺ and DPP4⁻ HA9M1 cells were sorted by flow cytometry and their gene expression compared to that of splenic T cells by Q-PCR. After initial analysis, the T cells and DPP4^{+/-}HA9M1 cells were cultured for 4 days using the same media and analyzed once again for Dpp4 expression. HA9M1: Hoxa9/Meis1-Ubiquitin-c-GFP (N=3 biological replicates from 2 independent experiments). Statistical significance was calculated by one-way ANOVA followed by Sidak's post-hoc test, error bars denote standard deviation.

FIG. 21A shows plots of DPP4 expression on AML cells, analyzed by cell surface staining and flow cytometry, after 3 day co-culture with stromal cells. BM stroma, immortalized bone marrow stroma from D.B.S.'s laboratory; MC 3_T3_E1, osteoblast precursor cell line; MLO_A5, murine long bone osteocyte cell line; MS-5, mouse stroma cell line; NIH-3T3, fibroblast cell line. (Control, NIH-3T3, MS-5 and BM stroma, n=6; MC_3T3 and MLO_A5, n=3 biological replicates; two independent experiments). Statistical significance was calculated using one-way ANOVA followed by multiple comparisons test. Error bars represent the standard deviation and are centered at the arithmetic mean.

FIG. 21B is a plot of the distribution of HA9M1 cells in D, M and R cavities on day 0, 1 and 3 after transplantation. Day 0: D0, n=22 cavities, M0 n=82 cavities, R0 n=41 cavities (6 independent mice); day 1: D1 n=21, M1 n=84, R1 n=43 cavities (6 independent mice); day 3: D3 n=16, M3 n=95, R3 n=21 cavities (7 independent mice). Error bars represent the standard deviation and are centered at the arithmetic mean. Statistical significance was determined using an unpaired, two-sided Mann-Whitney U-test. The 99.9% confidence intervals for P, IM and NP cells from FIG. 11B-11C are marked by red, blue and green rectangles, respectively. HA9M1, Hoxa9/Meis1-Ubiquitin-c-GFP; IsoT, isotype; MFI, mean fluorescence intensity.

FIG. 22A is a diagram of an example flushing system, according to aspects of the present disclosure. A 3D-printed holder secures two blunt-tipped needles on the objective lens. They are used to flush a continuous stream of PBS across the sample (in this case a mouse skull) during the laser ablation procedure.

FIG. 22B is a detailed diagram of an example flushing system, according to aspects of the present disclosure. A peristaltic pump is used to flow a continuous stream of PBS, saline, or water across the sample. Flow direction marked by red arrows.

FIG. 23 is a diagram of the micropipette assembly that is mounted on a sliding stage. The sliding stage has an IN (post IN and micropipette IN the microscope field of view), as well as an OUT position (post is OUT, along with the micropipette). It is created by removing the actuator from a translational stage.

FIG. 24 is a schematic of a bone thinning procedure. A razor blade is advanced parallel to the bone surface in a single direction to remove bone material. This step is repeated until the red bone marrow is visible and the desired bone thickness is achieved.

FIG. 25 shows photographs of unthinned (left) and thinned (right) tibia bone. The bone marrow has a redder hue after thinning. Note: PBS drops have been added to the bone to ensure sample integrity.

FIG. 26 is a cross-section of a multiphoton microscopy image from an unthinned (top) and thinned (bottom) tibia bone. Bone marrow cells cannot be resolved for an unthinned sample. Scale bar: 50 μm.

FIG. 27 is a photograph of a side-view of the bone mount. Region of interest is positioned parallel to the surface of the glass slide.

FIG. 28A is a schematic of a mouse head indicating an incision site (black lines). Typical dimensions are 7-9 mm in length and 3-5 mm across.

FIG. 28B is a photograph of a mouse skill. The positions of Lambda (L) and Bregma (B) are marked by arrowheads.

FIG. 29 is a diagram of the setup for ejecting the BM aspirate into a microfuge tube. After aspiration, the micropipette is removed from the sample using the sliding stage and the aspirated BM is ejected into a microfuge tube.

FIG. 30A is a photograph of the perfusion procedure. Skin is removed to aid in the visualization of the surgical procedure.

FIG. 30B is a photograph of the perfusion procedure. The needle is inside the left ventricle and the mouse is being perfused.

FIG. 31A is a diagram of an example of laser ablation and insertion of micropipette for cell aspiration. Placement of the microchannel at a 20 μm distance to the target cells, along with the inserted micropipette.

FIG. 31B is an image of a 3D representation of a 3D volume of bone after the laser ablation procedure. Bone signal is visualized by second harmonic generation of the incident wavelength.

FIG. 31C is a diagram is a schematic of a micropipette that has been inserted into the bone marrow, along with the air syringe used for cell aspiration.

### DETAILED DESCRIPTION

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

System Component

In one aspect, the disclosed system comprises an optical and mechanical apparatus that can perform both multiphoton and/or confocal imaging (at arbitrary frame rates) as well as plasma-mediated laser ablation. This makes it possible to aspirate/isolate cells with a micropipette under image guidance from small, spatially confined volumes of mouse bone marrow tissue (on the order of 50×50×50 μm, although the exact volume can be controlled by the device and can be significantly smaller or larger). Note that in tissues like the bone marrow, plasma-mediated laser ablation is required to perform a microsurgery procedure on bone and generate a small opening or access port to the bone marrow underneath. This enables insertion of the micropipette so that cells can be aspirated under image guidance. In other tissues the laser ablation procedure could be used to dissect out small tissue blocks. Further tissue types may not require laser ablation at all and cells could be aspirated directly by micropipette. In all the above settings, the three-dimensional (3D) spatial architecture is first visualized by multiphoton and/or confocal microscopy, the device then isolates cells (that could be viable or fixed) from a precise anatomical location. By imaging the tissue both before and after cell aspiration, the precise 3D spatial position of the isolated cell sample is determined. Through the analysis of these spatially-harvested micropipette samples, it is possible to study spatial variations in cell type, cellular function and cell-cell communication. In a disease setting such studies are important for the development of new therapeutic targets. One of the analysis tools (single-cell RNA-sequencing) is described in greater detail in the Examples below. It is this combination of cell harvest by micropipette under image guidance with single-cell RNA-sequencing that defines a new technology for spatially-resolved transcriptomics.

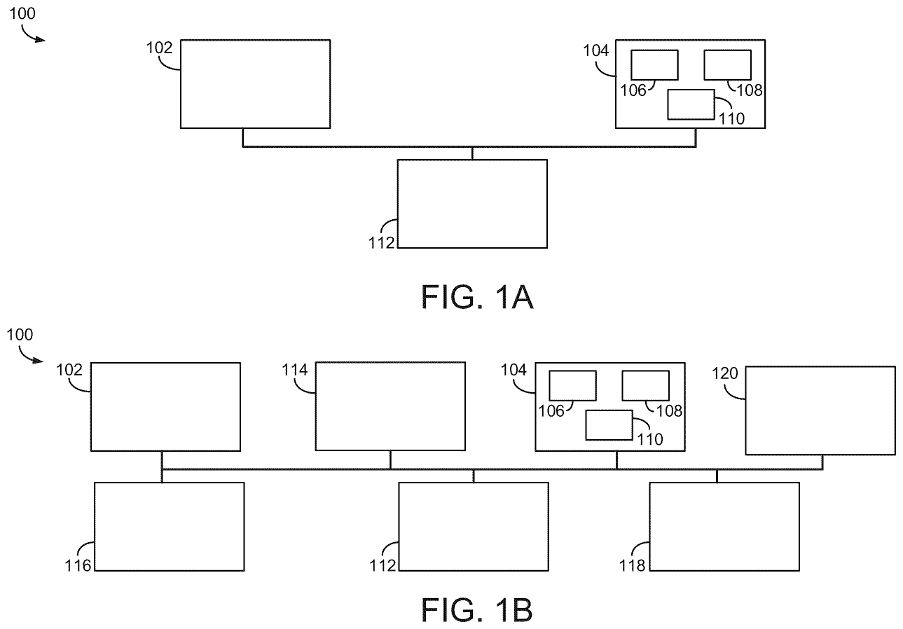

As illustrated in FIG. 1A, the system 100 comprises an optical microscope 102. The optical microscope 102 may be a confocal or multiphoton microscope. The system 100 further comprises a micropipette assembly 104 configured to aspirate a sample using a micropipette 106. In a non-limiting example, the micropipette assembly further comprises a tube 108 and vacuum pump 110 configured to connect to the micropipette for aspirating and ejecting samples. The vacuum pump 110 includes, but is not limited to, an air syringe. The system 100 further includes a processor 112 in communication with the optical microscope 102 and the micropipette assembly 104. As will be described in more detail below, the processor 112 may control the optical microscope 102 to image a subject and identify a region of interest in a target anatomy. In a non-limiting example, the anatomy may include, but is not limited to, organs, other soft tissue, bones, etc. For example, where the target anatomy is a bone, the region of interest may include a bone marrow cavity.

Further, the processor 112 may identify the region of interest from the images generated from the subject. In a non-limiting example, the region of interest includes at least on cell of a target population of cells. As will be described in the Examples below, the target population of cells may include bone marrow stromal cells or acute myeloid leukemia (AML) cells in the bone marrow cavity of a bone. In a non-limiting example, the target population of cells include a label that is identifiable using the optical microscope 102. For example, the label may include a fluorescently tagged molecule that is expressed by or selectively bound to the target population of cells. Examples of labels are provided in the Example section below. In a non-limiting example, the micropipette tip is also coated with a label for visualization under guidance of the optical microscope.

In a non-limiting embodiment, the optical microscope may be further integrated with an ablation laser 104. The ablation laser 104 can be the same as the multiphoton imaging laser. However, it should have enough power to generate a plasma in the microscope's image plane (which requires a pulse energy of at least 10-20 nJ). Optimal pulse energies will vary depending on the exact properties of the optical system (including laser repetition rate and pulse length), as well as the tissue type and exact application.

In a non-limiting example, the processor 112 is further configured to control the micropipette assembly for inserting the micropipette 106 into the region of interest.

As shown in FIG. 1B, the system 100 may further include an ablation laser 114. In a non-limiting example, the ablation laser 114 is configured to transmit an ablation beam towards the target anatomy under control of the processor 112. In one embodiment, the ablation laser 114 may be integrated with the optical microscope 102. For example, the ablation laser beam may be generated by a multiphoton microscope.

In a non-limiting example, the ablation laser emits the ablation beam at a repetition frequency range of 1 kHz to 10 MHz.

In a non-limiting example, the ablation laser emits the ablation beam at a pulse energy in a range of about 5 nJ to 1 μJ.

In a non-limiting example, the ablation laser emits the ablation beam at a pulse duration of less than 1 ps.

As will be described in further detail below, the ablation laser 114 may be used to generate an opening in the target anatomy. For example, to access the target population in a bone marrow cavity, it is necessary to form a channel in the bone configured for insertion of the micropipette. The ablation laser 114 may alternatively or additionally be used to ablate the tissue in the region of interest.

The system 100 in FIG. 1B may further include a flushing system 116. The flushing system may be installed on an objective lens of the confocal or multiphoton microscope 102. In a non-limiting example, a holder secures two blunt-tip needles opposite one another. In this example setup, the efflux from one needle is the influx to the other needle, such that a steady stream of PBS or saline flows across the sample during the ablation procedure and remove any gas and debris. In a non-limiting example, flow is achieved using a dual channel peristaltic pump with tubing, wherein the needles are attached to one end of the tubing.

In another non-limiting example, the system 100 in FIG. 1B further includes a microcontroller 118 configured to move the micropipette in and out of the region of interest for aspiration under guidance of the optical microscope 102. In one example, the microcontroller 118 may be operated by the processor 112 for precise positioning of the micropipette 106. Alternatively, the microcontroller 118 may be manually operated.

In another non-limiting example, the system 100 in FIG. 1B further includes a sliding stage 120 for translating the micropipette assembly 104 in a coordinate frame of reference relative to a sample stage (not shown). In one example, the microcontroller 120 may be operated by the processor 112. Alternatively, the microcontroller 118 may be manually operated.

In a non-limiting example, the system 100 is modular and may include any number and combination of the elements described above.

Method

Single-cell RNA sequencing of spatially-harvested cell samples using the Image-seq device described above represents a new technology for spatial transcriptomics. Spatial transcriptomics includes several commercially available technologies (for example, 10× genomics' Visium or Vizgen's Merscope platform), as well as a number of technologies that are still in development and not yet commercially available (for example Slide-Seq). Because of Image-seq's ability to isolate intact (viable or fixed) cells it can be combined with state-of-the-art library preparation kits and protocols that are commercially available. When combined with Smartseq library preparation it has a higher sensitivity (number of genes that are detected per cell) than any currently available spatial transcriptomics technology. Image-seq can also be used to study rare cells that would not be detected by other spatial transcriptomics technologies (see Example 1 below) and does not require the generation of tissue sections, which is a limitation of commercially available technologies. Image-seq may be combined with state-of-the-art multi-omics analysis tools like (single-cell) proteomics, metabolomics, ATAC-seq and DNA sequencing. Combinations of the device with these single-cell analysis tools represent a new technology for spatially-resolved single-cell multi-omics.

The advantages of Image-seq include the combination of multiphoton/confocal microscopy with spatially-resolved single-cell RNA sequencing, the method's high sensitivity, its applicability to rare cells and stromal cells, as well as its potential for multi-omics analysis. In addition, it does not require the generation of tissue sections, making it the only spatially-resolved single-cell RNA sequencing technology that can currently be applied to bone marrow tissue.

As described in the Example section below, the systems and methods disclosed herein generate high-quality single-cell RNA sequencing data. It has been shown that it can be used to study hematopoietic cells, stromal cells, rare cancer cells, and that it provides higher sensitivity than any currently available spatial transcriptomics technology. It has also been demonstrated that it doesn't require the generation of tissue sections and can be used in conjunction with other single-cell analysis techniques (i.e. flow cytometry and microfluidic cell sorting).

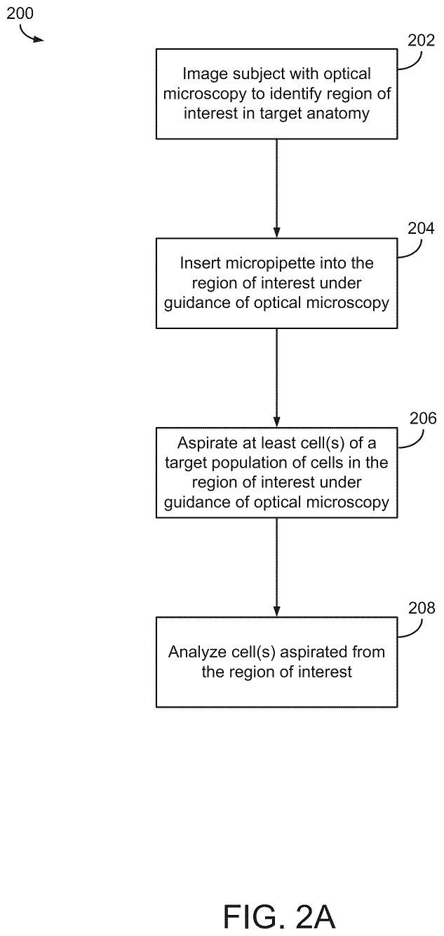

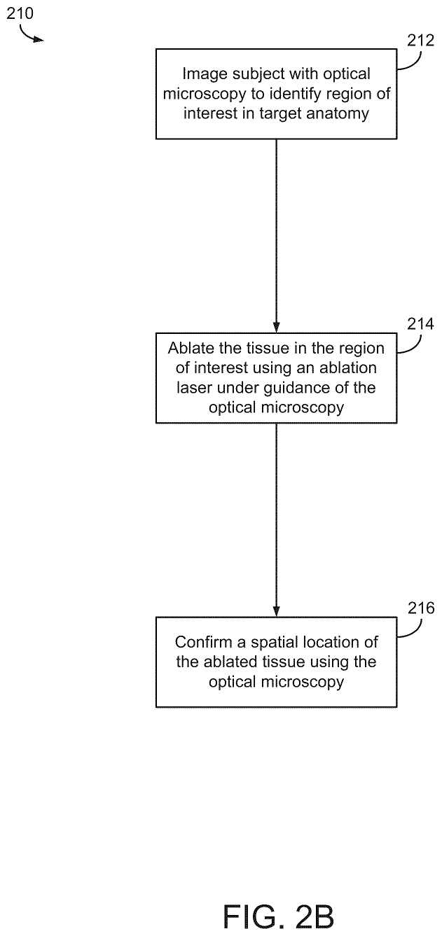

In another aspect of the present disclosure, a method of identifying and aspirating target cells is disclosed. The method illustrated in FIG. 2A may be performed using any of the system embodiments described above in FIGS. 1A-1B.

The method 200 of FIG. 2A includes imaging a subject using optical microscopy to identify the region of interest in a target anatomy at step 202. At step 204, a micropipette is inserted into the region of interest under guidance of the optical microscopy. Next, at step 206, the method involves aspirating at least one cell of a target population of cells in the region of interest under guidance of the optical microscopy. At step 208, the aspirated cell(s) from the region of interest are analyzed.

In a non-limiting example analyzing the aspirated cells at step 208 includes, but is not limited to, spatially resolved transcriptomic analysis, single cell RNA sequencing, bulk RNA sequencing, single-cell multi-omics analysis, bulk multi-omics analysis, genetic manipulation of aspirated the cell(s), transplantation of the aspirated cell(s), and cell colony formation. The Example sections below provide disclosure for single cell RNA-sequencing and spatially resolved transcriptomic analysis.

In a non-limiting example, multi-omic analysis may be performed using droplet based single cell multi-omics sequencing or multi-omic analysis with flow cytometry sorting.

In a non-limiting example, transplantation may involve transplanting spatially harvested sample into irradiated or non-irradiated recipients. The cells of the samples may or may not be (i) sorted prior to transplantation using either a flow cytometer or microfluidic cell sorter and (ii) genetically manipulated (for example gene knockout or gene knockdown) prior to transplantation. Such studies may aid in revealing functional differences between cells from different spatial locations.

In a non-limiting example, colony formation assays involve in vitro culturing of spatially-harvested samples (as described in the Example section below).

In another aspect of the present disclosure, a method of ablating tissue is disclosed. The method illustrated in FIG. 2B may be performed using any of the system embodiments described above including the ablation laser 114.

The method 210 of FIG. 2B includes imaging a subject using optical microscopy to identify a tissue in the region of interest in a target anatomy at step 212. At step 214, the tissue in the region of interest is ablated using an ablation laser under guidance of the optical microscopy. Next, at step 216, the spatial location of the ablated tissue is confirmed using the optical microscopy. In a non-limiting example, the tissue that was identified for ablation, such as by tissue-specific labeling as previously described, can be confirmed for successful ablation by finding an absence of the tissue-specific labeling in images generated from optical microscopy.

In a non-limiting example, method 210 of FIG. 2B may additionally include creating an opening in the target anatomy (for example bone) using ablation, to provide access to the tissue region of interest for cell aspiration. For example, the ablation laser may transmit an ablation beam toward the bone after imaging the subject to create a channel in the bone.

In a non-limiting example, methods 200 and 210 of FIGS. 2A-2B, respectively, may further involve dissecting the target anatomy from the subject before imaging steps 202, 212 or micropipette insertion step 204 or ablation step 214. As will be described in further detail in Example 2 below, a long bone may be dissected from a subject and processed for cell aspiration or tissue ablation.

It will be appreciated by those skilled in the art that while the disclosed subject matter is described above and in the examples below in connection with particular embodiments and examples, the invention is not necessarily so limited, and that numerous other embodiments, examples, uses, modifications and departures from the embodiments, examples and uses are intended to be encompassed by the claims attached hereto. Each article cited herein is incorporated by reference in its entirety.

### EXAMPLES

Various features and advantages of the invention are set forth in the following examples.

### Example 1

The Image-seq methods and system described above have been used to study the bone marrow, the primary site of hematopoiesis, where hematopoietic stem cells give rise to all of the blood cells in the body. The bone marrow is also the site where malignant cells can either originate (such as leukemia) or preferentially metastasize to (such as prostate or breast cancer). Although the functional organization of hematopoietic cells in the bone marrow has been extensively characterized, their 3D spatial organization has been difficult to assess due to their complexity and location deep inside the bone matrix. To demonstrate Image-seq's versatility and high sensitivity, high-throughput, droplet-based sequencing was used with the 10× Chromium platform to profile BM hematopoietic cells, as well as the Smartseq v4 protocol (Takara Bio USA, Inc., Mountain View, USA) to profile rare (<0.01% leukemic burden) acute myeloid leukemia (AML) cells and bone marrow stromal cells. Specifically, AML progression was tracked using intravital microscopy, and observed pronounced spatial heterogeneity in the earliest stage of expansion from single AML cells seeded in the bone marrow. This highlighted the need to capture these cells from distinct bone marrow locations where they either proliferate or remain quiescent, in order to identify the factors that regulate leukemia progression in the earliest stage of the disease development. Using this approach, DPP4 was identified as a key upregulated gene in AML cells from the more proliferative bone marrow compartments. Strikingly, DPP4 was not expressed on the same cells cultured in vitro, suggesting that DPP4 was specifically activated in vivo and was correlated with disease progression.

Results

Cell Isolation Under Image Guidance

The core of the system is a confocal or multiphoton microscope with an additional laser beam capable of tissue ablation. The requirement for the ablation laser is that it needs to have sufficient pulse energy (>10 nJ per pulse, or approximately 10-fold the pulse energy typically used in multiphoton microscopy) to generate a plasma through multiphoton ionization at the laser focus. To minimize thermal damage, it also needs to have a low average power, which can be accomplished by reducing the pulse repetition frequency from the ˜80 MHz typically used for multiphoton microscopy to a few MHz to compensate for the higher pulse energy needed for tissue ablation. These requirements may be readily fulfilled with commercial or industrial femtosecond fiber lasers for micromachining or ophthalmic microsurgery. The plasma is used to ablate or etch away bone with minimal collateral damage to the surrounding tissue. A small, ˜50×100 μm channel is created, through which a micropipette, controlled by a micropipette assembly or micromanipulator, is inserted to aspirate live bone marrow cells under image guidance. The sample is expelled from the micropipette, which directly generates a single-cell suspension.

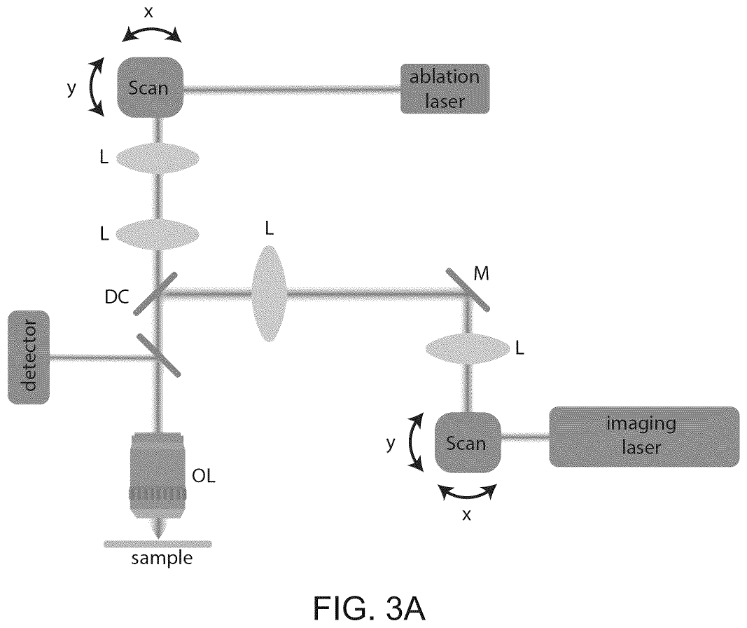

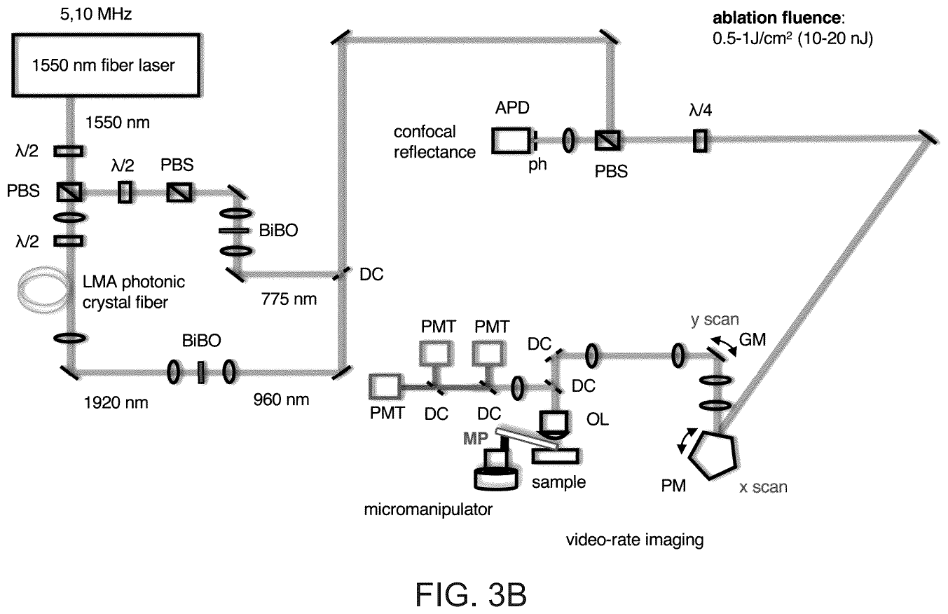

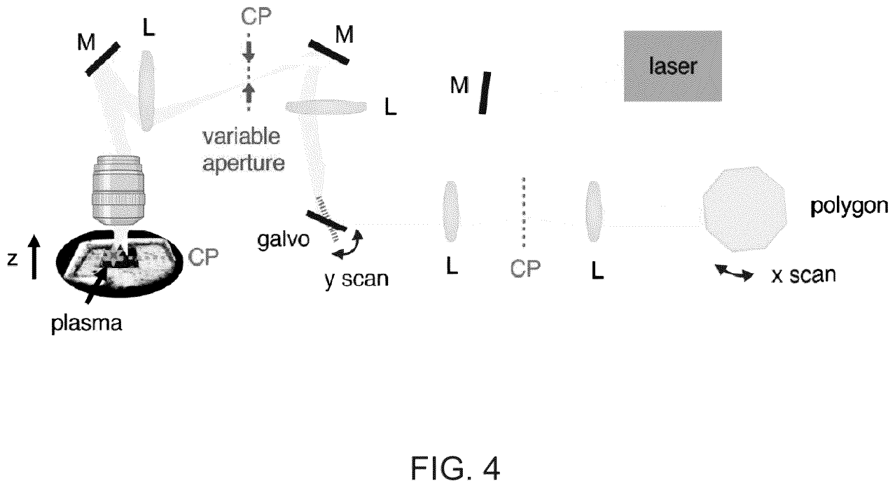

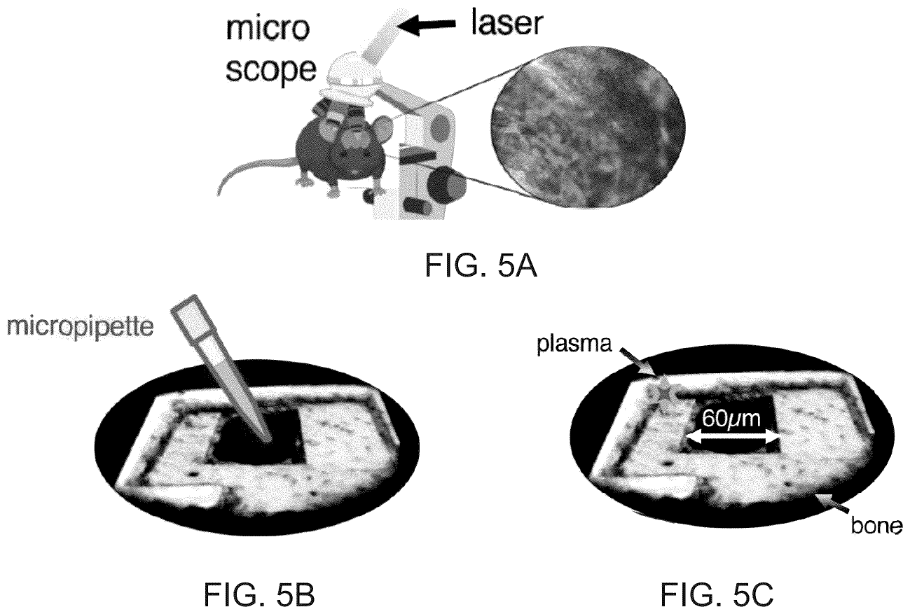

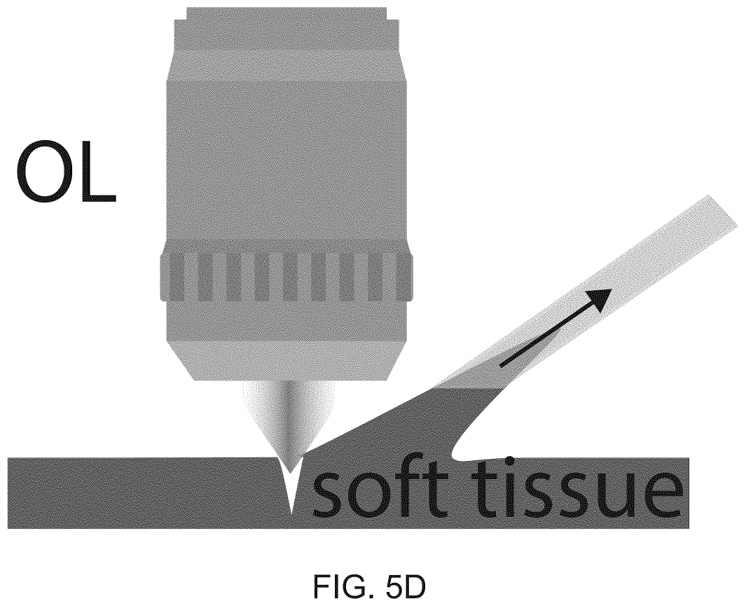

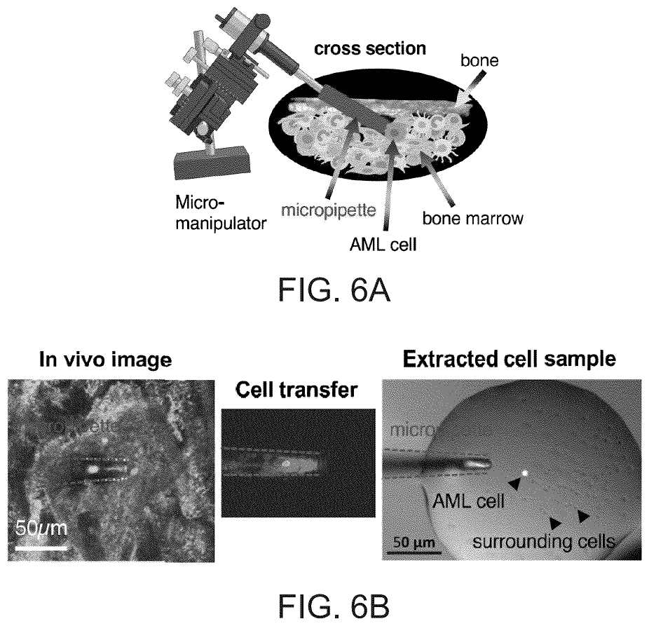

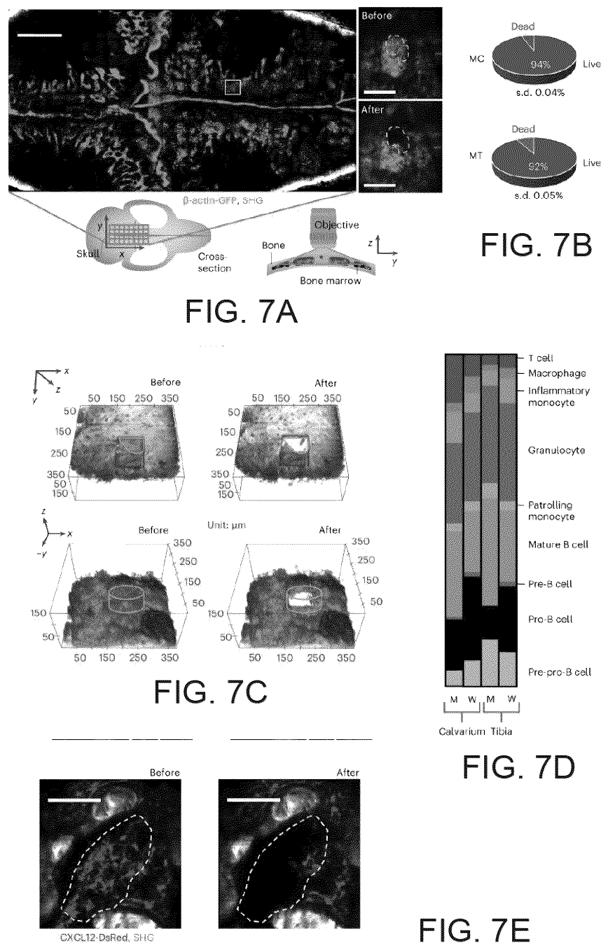

In the implementations presented herein, the imaging arm and the ablation arm are powered by a single femtosecond fiber laser operating at 5 MHz repetition frequency. This repetition frequency enables full-field (500×500 pixels) image acquisition at 15 frames per second (half the video rate) with a single laser pulse per pixel. However, it is also possible to integrate an ablation capability into an existing confocal or multiphoton microscope by adding an ablation laser that fulfills the requirements described above. Detailed optical designs are shown in FIGS. 3A-3B. In FIG. 3B, the output of a 1550 nm femtosecond fiber laser is frequency-doubled to 775 nm and used for both imaging and plasma-mediated laser ablation by adjusting the pulse energy between 1-20 nJ. An additional imaging wavelength that is tunable between 940-980 nm is provided through soliton generation in a photonic crystal fiber. A revolving polygon and scanning galvo mirror, conjugated to one another and the back aperture of the objective lens, steer the ablation/imaging beam across the field of view in the imaging plane (see FIG. 4). A variable aperture at the intermediate image plane controls the 2D ablation geometry along the x and y dimensions (see FIGS. 5A-5C) while the sample is translated along the z dimension to image or ablate a 3D tissue volume. By etching through bone, a small opening (˜50 μm×100 μm) is created, and a micromanipulator is used to insert a fluorescently coated micropipette through the opening at a 20° angle into the bone marrow with micrometer spatial precision (see FIGS. 5A-5C). Prior to making the opening, a larger area of bone is thinned (˜200×300 μm), improving micropipette accessibility, as well as imaging depth. This step takes ˜3 min. Target bone marrow cells are aspirated into the micropipette by suction with an Air Syringe, with the displaced volume controlling the total number of aspirated cells (from a few to a few thousand) (FIGS. 6, 7C, 7E). With this design, bone marrow cells were aspirated from freshly-collected bone samples as well as from live animals while tracking the process in real time. FIGS. 6A-6B show the bone marrow aspirate containing the target cell from the in vivo image after transfer to a glass slide and visualization with a wide-field fluorescence microscope. Using this strategy, recovery of a single, viable target cell has an efficiency ˜70%.

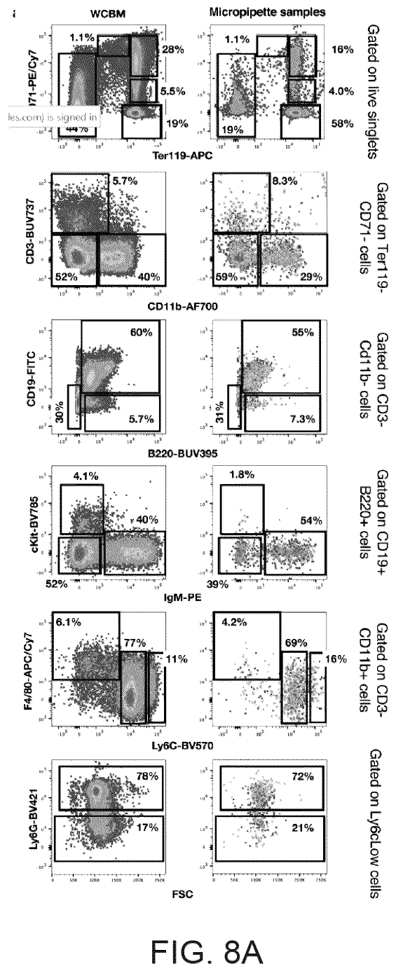

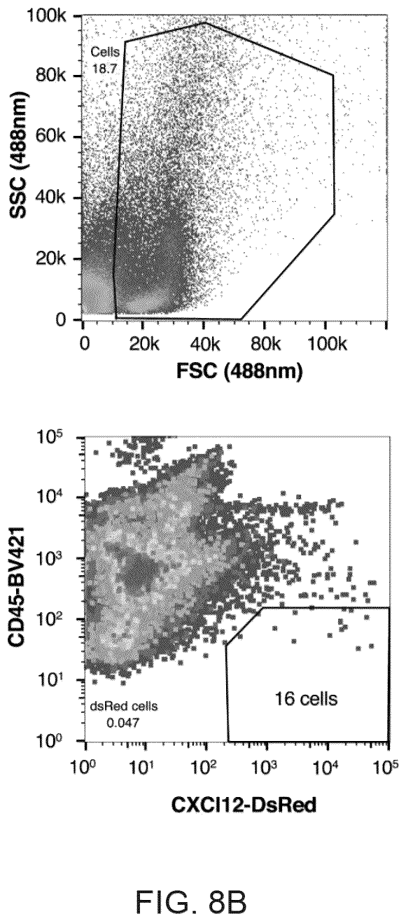

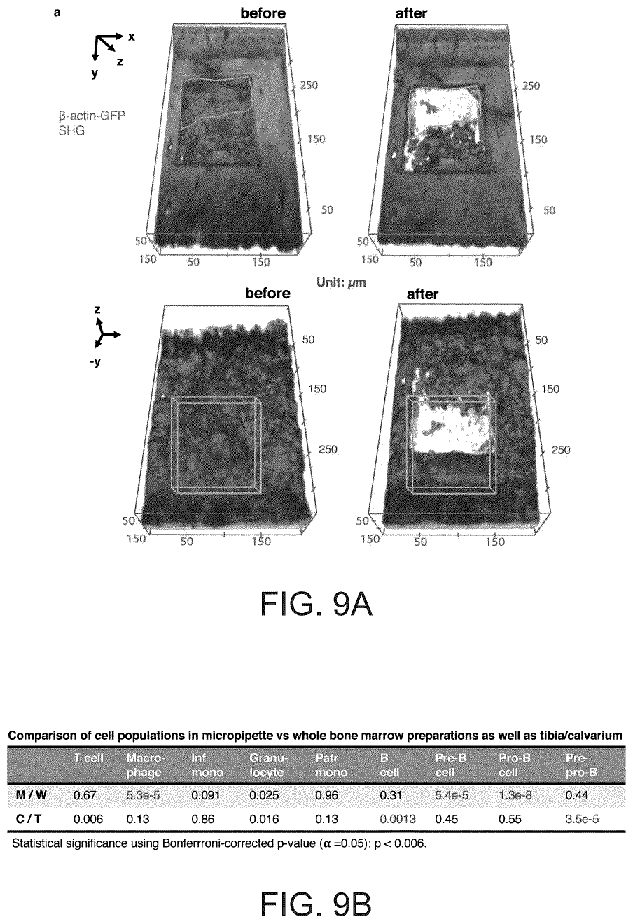

To further demonstrate the system's ability to isolate live cells from defined anatomic regions of murine bone marrow, it was used to isolate bone marrow samples from β-actin-green fluorescent protein (β-actin-GFP) mice. FIG. 7A shows a 4×8 mm, tiled, maximum intensity projection image of the calvarium bone marrow, along with a region of interest from which cells were aspirated both before and after cell extraction. To minimize sample loss, post-extraction sample processing was eliminated by performing transcardial perfusion prior to cell isolation. This reduced the amount of red blood cells in the samples, and a single cell suspension was directly generated by expelling samples from the micropipette. Note that there is a fair amount of variation in the CD71−Ter119+ cell compartment that is attributed to slight differences in perfusion quality (FIG. 8A). A total of nine calvarial bone marrow and four tibia bone marrow micropipette samples, respectively, containing an average of 2,000 cells each, were isolated from a total of five mice, yielding an average viability of 94% and 92%, respectively (FIG. 7B). A 3D visualization of a representative volume of calvarial bone before and after cell extraction is depicted in FIG. 7C. Similarly, FIG. 9A shows a representative volume of tibia bone marrow both before and after cell extraction. Both the view from above the bone surface and that achieved by rotation of 180° around the x axis are shown. Additionally extracted cell samples were analyzed for their cell surface marker expression by flow cytometry and were able to detect the major hematopoietic cell populations in the pooled micropipette samples with similar proportions to those in whole bone marrow control samples (see FIGS. 7D, 8A and 9B). Generally, the cell proportions were in good agreement, although statistically significant differences were observed between tibia and calvarium bone marrow for the B-cell and pre-pro-B-cell populations. Significant differences were also observed between the micropipette and whole bone marrow preparations for macrophage, pre-B-cell and pro-B-cell populations. It is unclear whether the differences between micropipette and whole bone marrow preparations are due to technical effects or the fact that micropipette samples were typically harvested from within ˜80 μm of the endosteal surface.

By perfusing with enzymatic digestions buffer after the initial perfusion (to reduce the number of red blood cells) and incubating the bone sample for −20 min, a protocol for the isolation of stromal cells was established. FIG. 7E shows a maximum intensity projection image of a volume of CXCL12 (C-X-C motif chemokine ligand 12)-DsRed-expressing bone marrow cells both before and after cell extraction. FIG. 8B shows the gating strategy used for quantification of CD45⁻CXCL12⁺ stromal cells in the micropipette samples: 3-4 cells in a typical aspirate were detected.

Validation of Image-Seq Technology

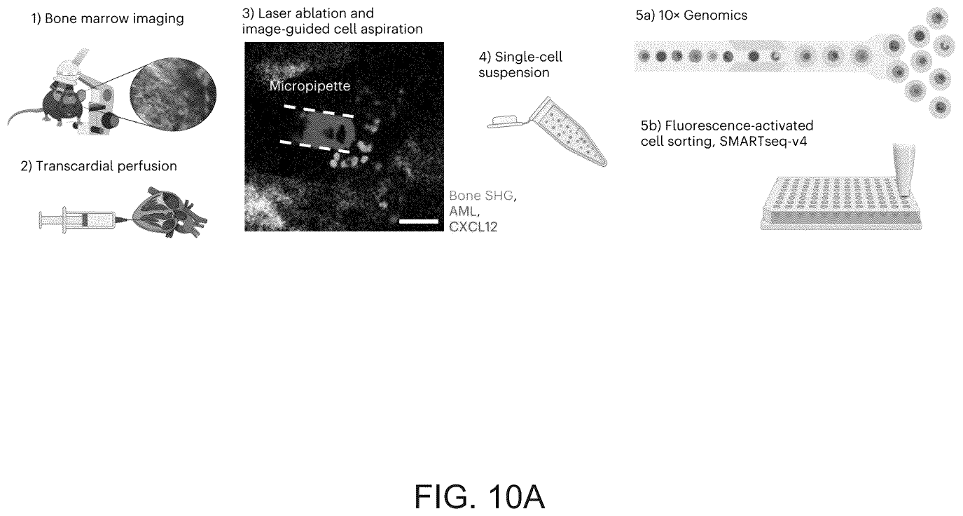

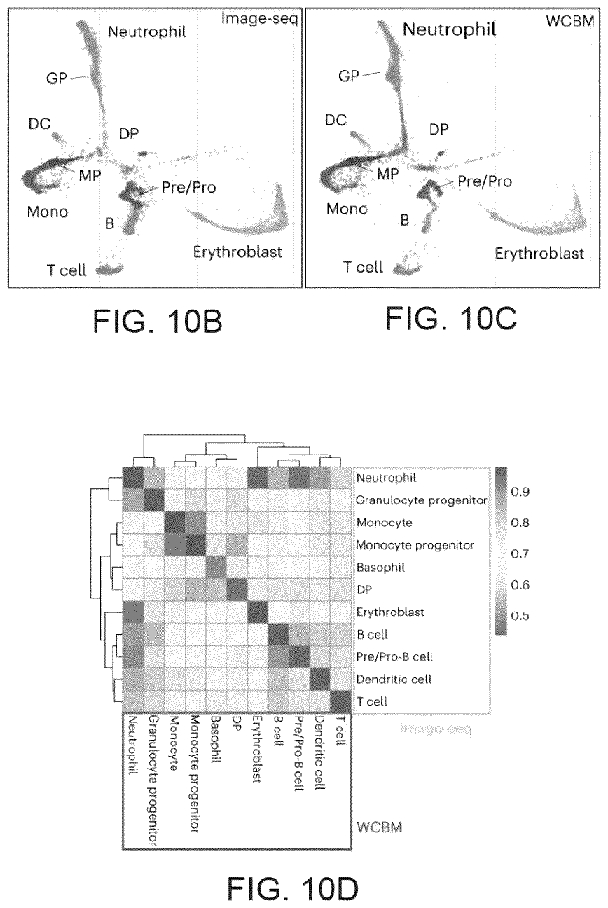

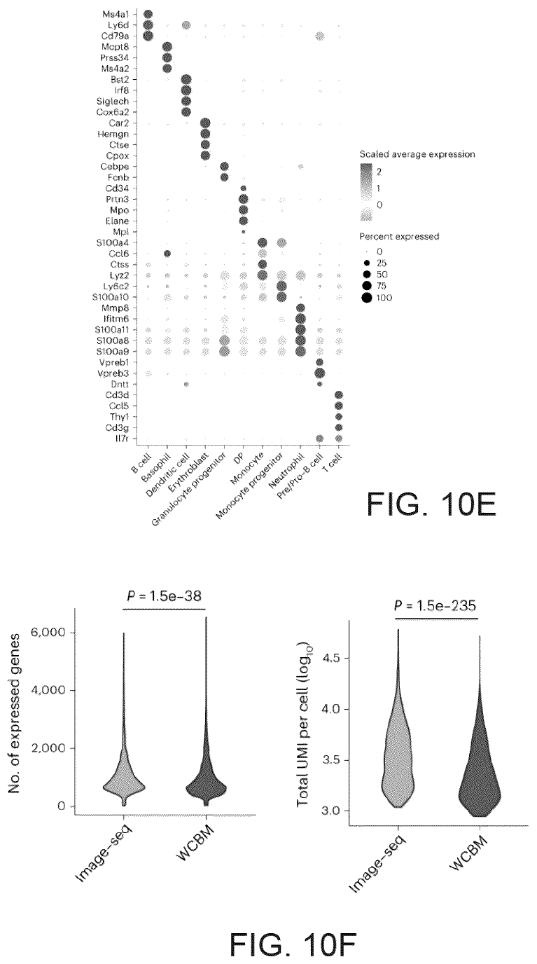

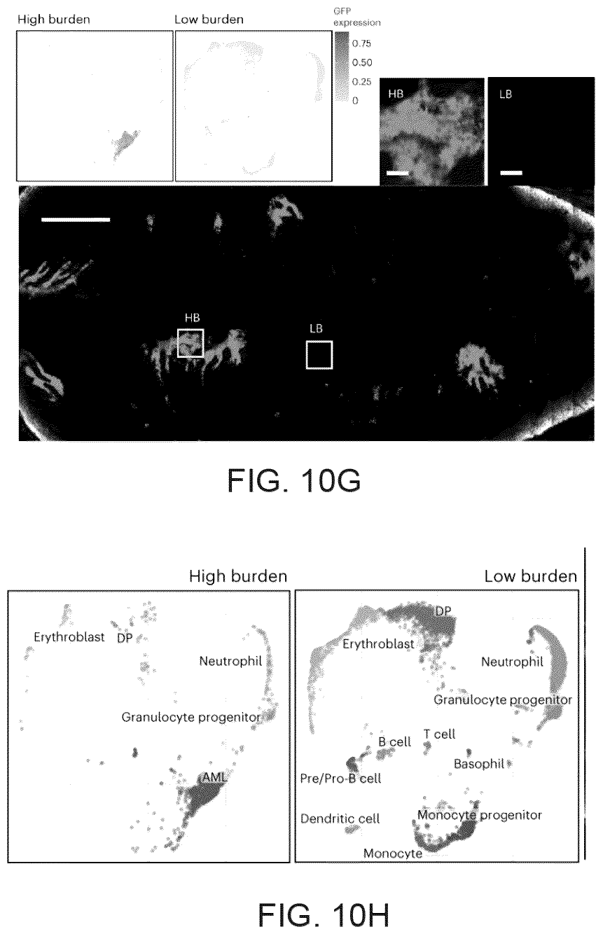

An outline of the Image-seq workflow is shown in FIG. 10A. Regions of interest (ROIs) are identified by multiphoton or confocal imaging, mice are perfused, and laser ablation, combined with cell aspiration by micropipette, is then used to isolate cell samples. The entire procedure takes ˜20 min per location.

A single-cell suspension is generated by expelling the sample from the micropipette into a tube that is transferred directly to the 10× Chromium chip for high-throughput characterization of the entire cell sample. Alternatively, samples can be stained, and individual cell populations sorted into wells by flow cytometry, for subsequent library preparation and sequencing using the Smartseq-v4 protocol. To validate the technology, 11 Image-seq samples from a total of n=5 C57B16 mice were collected, along with three whole calvarium bone marrow (WCBM) samples (n=3 mice) for single cell isolation and library preparation using the 10× Chromium platform. After sequencing and read alignment, the Conos package in R was utilized to integrate the multiple scRNA-seq datasets and align them with other public scRNA-seq data. Leiden clustering was used to determine joint cell clusters across the entire dataset, identifying most major hematopoietic cell populations, and visualizing them by uniform manifold approximation and projection (UMAP) embedding (FIGS. 10B-10C). The population at the plot center expresses multi-lineage progenitor markers such as CD34, C/EBPa, Hoxa9 and Meis1, as well as genes associated with granulocytic and monocytic priming (MPO, Elane, Ctsg, Prtn3), and with megakaryocytic priming (Mpl, Mycn), and was therefore termed ‘diverse progenitors’. From here, lymphoid (marked by expression of Vpreb1-3, Rag1-2, Cd19, Dntt, Mzbl, Ebf1), erythroid (marked by expression of Car1-2, Hemgn, Ctse, Cpox, Smim1), granulocytic (Cebpe, Fcnb, S100a8, S100a9, Camp) and monocytic (Irf8, Klf1, F13a1, S100a4, S100a10, Ly6c2) lineages branch off in different directions. Dot plots of selected marker genes for each cell population are shown in FIG. 10E. A list of differentially expressed genes within each cluster is included in Table 51. Here 11 Image-seq samples were pooled to characterize the distribution of cell populations in the limit of a large number of samples (for which the same distribution as for WCBM samples were expected if there were no technical differences between the two sampling strategies).

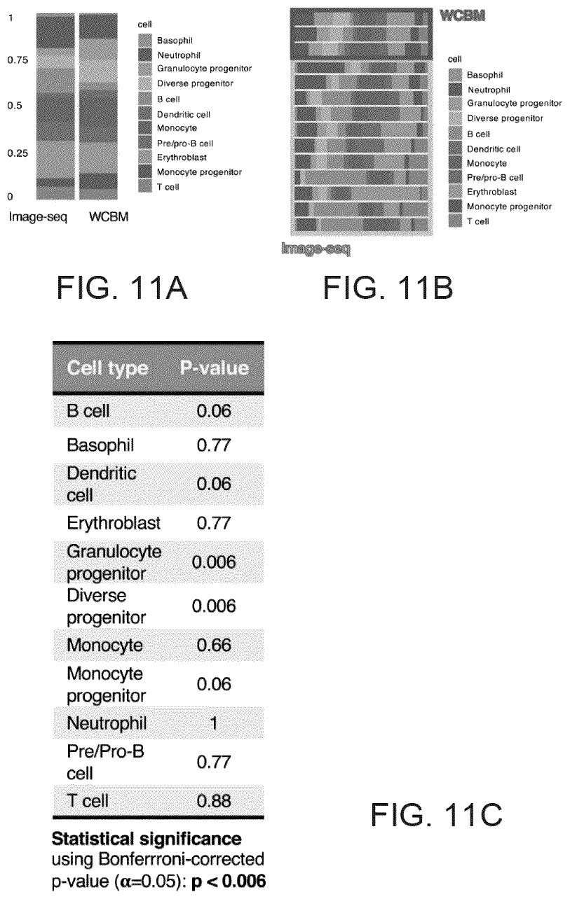

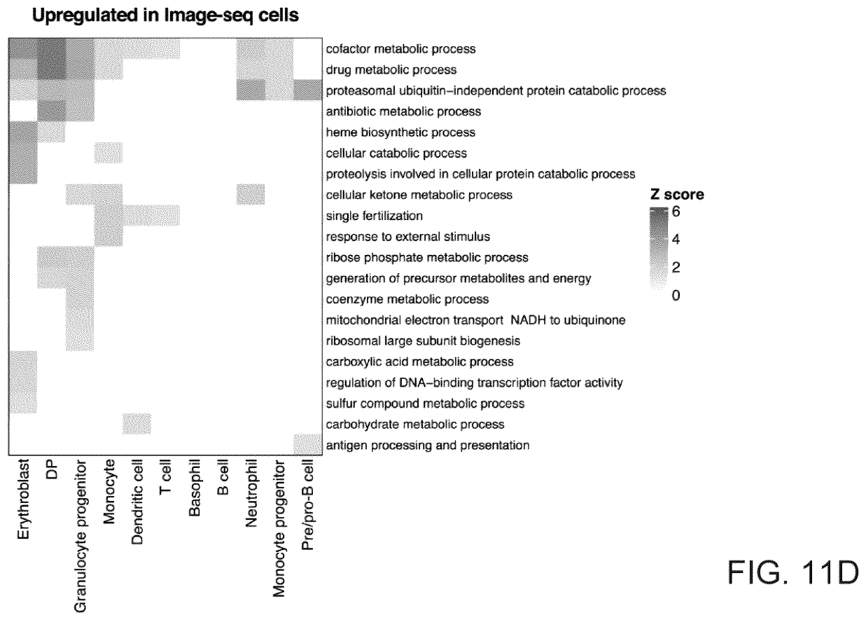

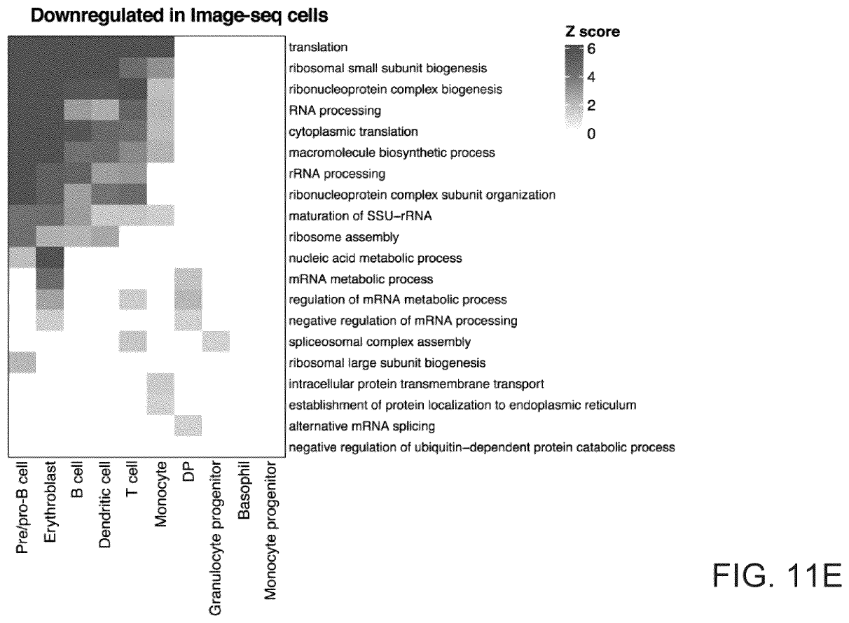

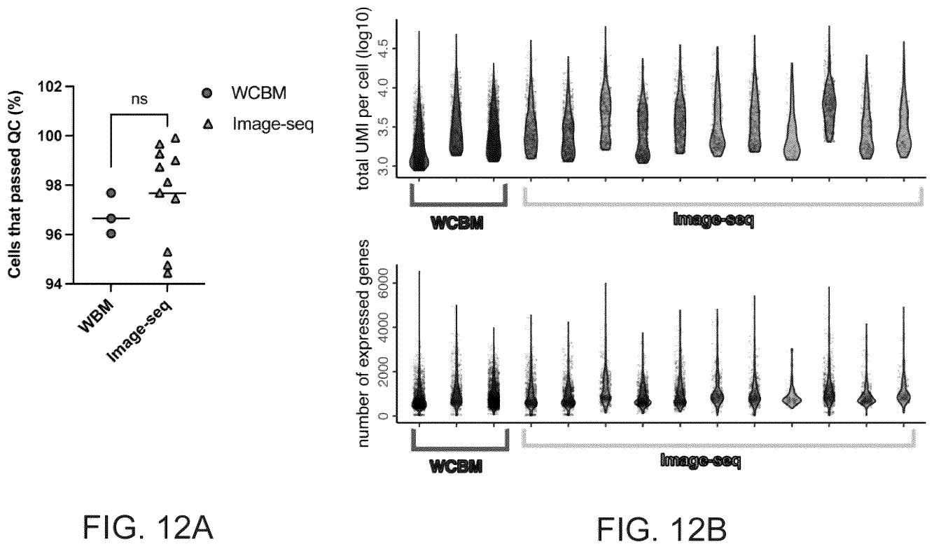

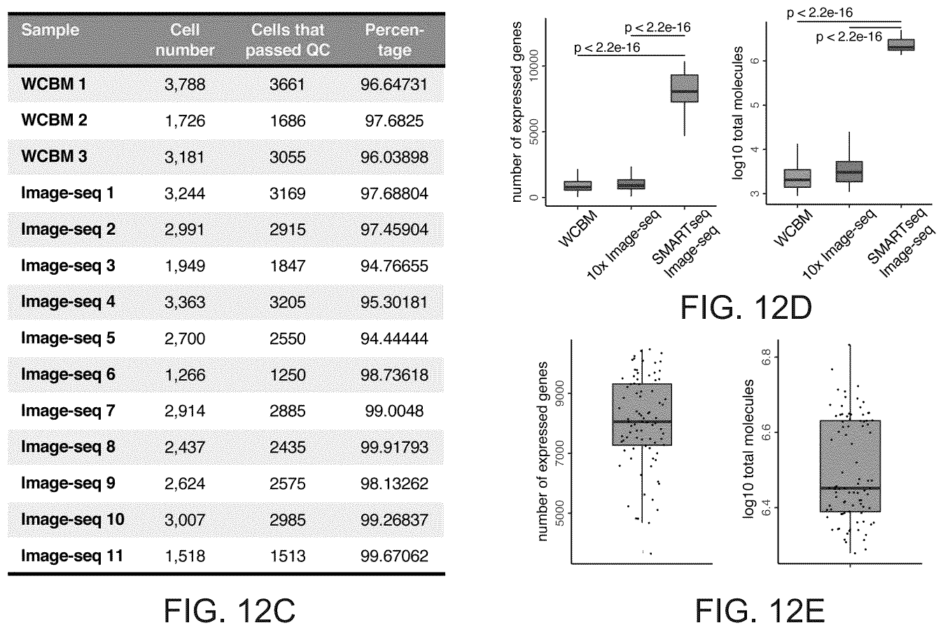

As expected for sampling small volumes from different locations, significant sample-to-sample variations were observed (a detailed breakdown is given in FIG. 11B for a detailed breakdown), no statistically significant differences were observed in the proportion of any cell populations when comparing Image-seq (FIG. 10B) and WCBM (FIG. 10C) preparations in aggregated form (FIG. 11C). The transcriptional similarity of the major cell types from WCBM and Image-seq samples were further examined. Correlation coefficients of average gene expression levels between WCBM and Image-seq (FIG. 10D) samples demonstrate a strong correlation between individual cell populations collected using the two methodologies. Gene ontology analysis of differentially expressed genes between Image-seq and WCBM samples for the most part showed very general terms that were not associated with cell damage or cell stress (FIGS. 11D-11E), although there was a notable downregulation of ribosomal proteins and mitochondrial transcripts in Image-seq samples, which could indicate lower cell stress and could be related to the overall higher number of unique molecular identifiers and genes per cell that were observed in the Image-seq samples (FIG. 10F). Additionally, the percentage of cells that passed quality control were assessed using the two experimental strategies (FIG. 12A) and found no statistically significant differences. The distribution of unique molecular identifiers and genes per cell for each sample collected were plotted (FIG. 12B), along with a table that summarizes the overall number of cells in each sample and the ones that passed quality control (FIG. 12C). Again, although some variations were observed between samples, the data as a whole led to the conclusion that Image-seq produces scRNA-seq data with a quality that is comparable to or exceeds that of conventional cell harvesting protocols.

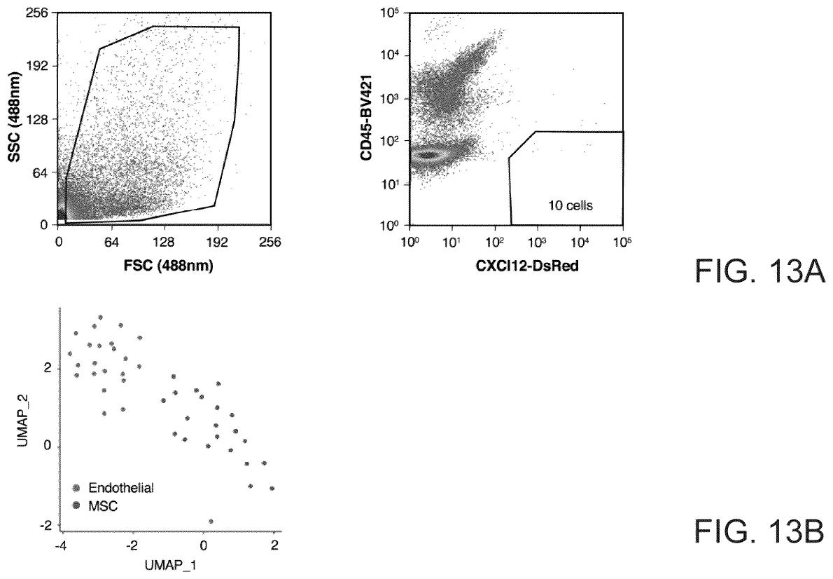

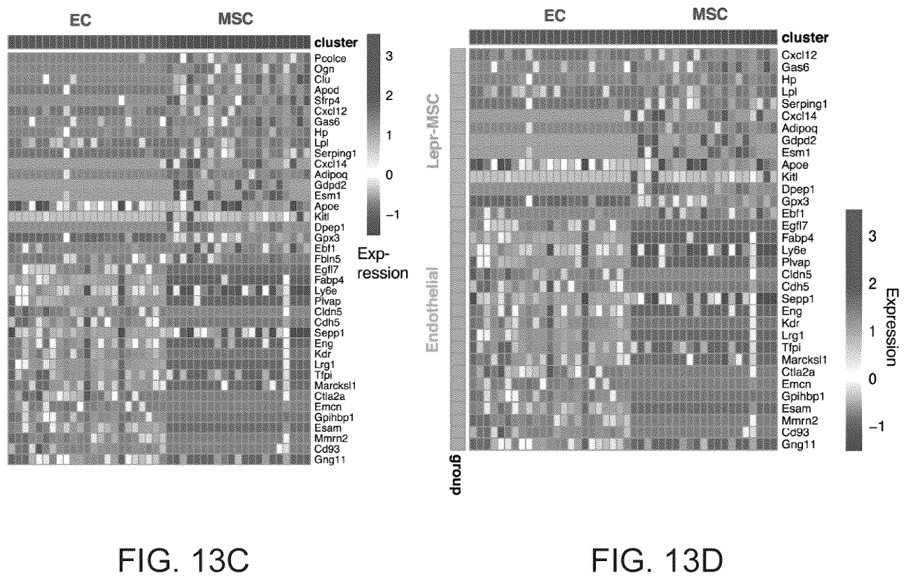

To demonstrate the technology's ability to obtain spatially-resolved single-cell transcriptional data of stromal cells (FIGS. 13A-13D), 43 CD45⁻CXCL12⁺ cells were isolated by micropipette and flow cytometry. After library preparation and sequencing using SMARTseq-v4, a shared nearest neighbors graph and UMAP embedding of these cells were constructed (FIG. 13B), identifying two separate clusters. These were annotated as mesenchymal stromal and endothelial cells (FIG. 13D) based on literature marker genes. A heatmap showing differentially expressed genes within these clusters is shown in FIG. 13C.

To further validate the spatial selectivity of the technology, an MLL-AF9 mouse model of acute myeloid leukemia (AML) was imaged in which leukemia cells express GFP under the ubiquitin promoter at day 10 after transplantation. As shown previously, regions of high leukemic burden were found to be interspersed with regions of low leukemic burden (FIG. 10G). Image-seq was used to isolate cells from a high and low-burden region, and were processed separately for scRNA-seq using the 10× Chromium protocol. UMAP embeddings of both samples demonstrate that the high burden sample was composed primarily of AML cells, whereas the low-burden sample did not include leukemia cells (FIG. 10H). Furthermore, strong levels of Gfp expression were detected in the AML cell cluster from the high burden sample (FIG. 10G), highlighting the technique's spatial selectivity and high sensitivity.

Image-Seq Analysis of Early Leukemia Expansion

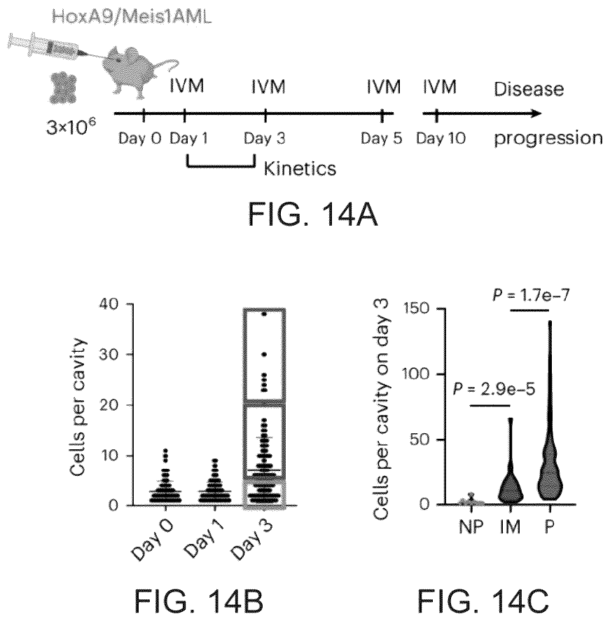

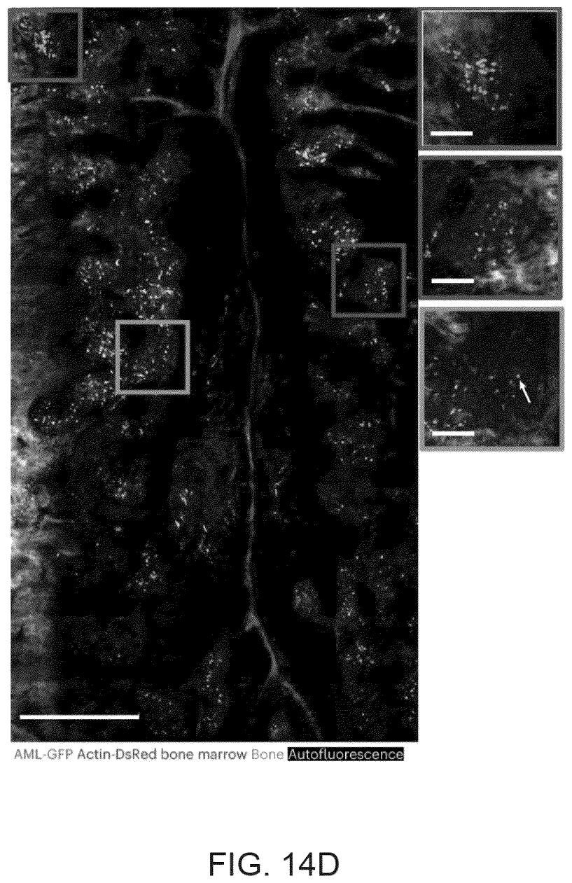

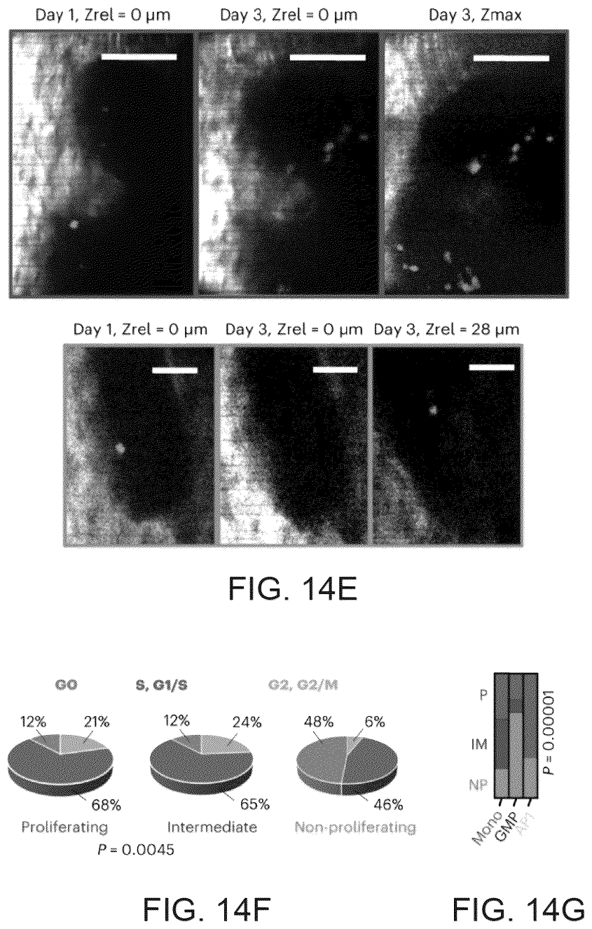

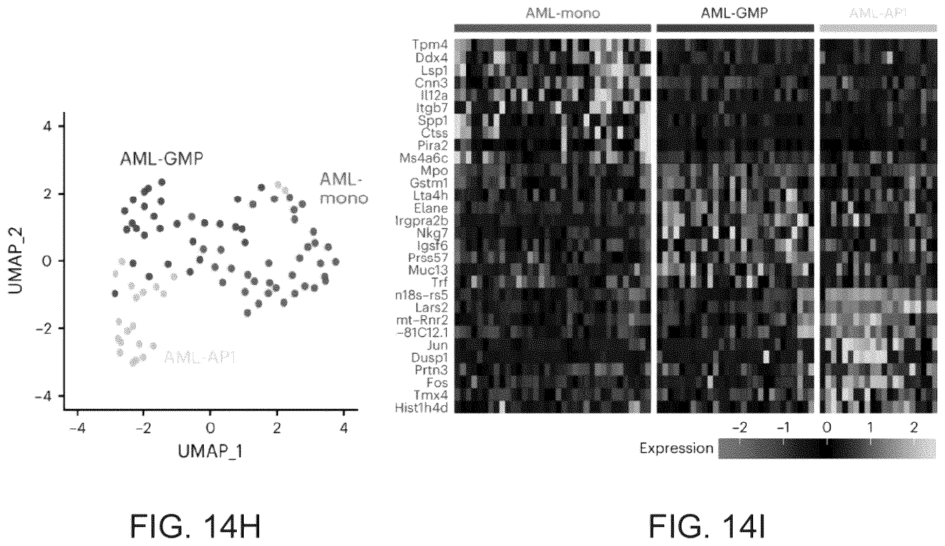

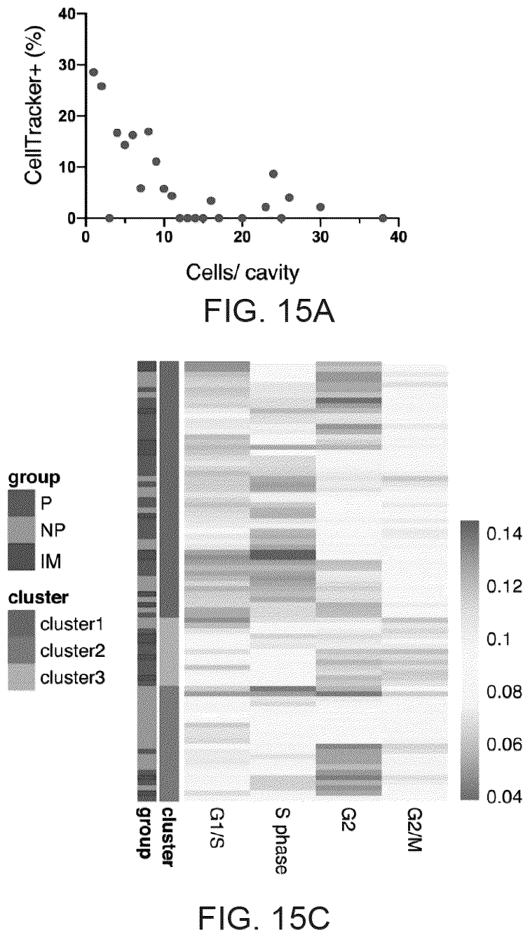

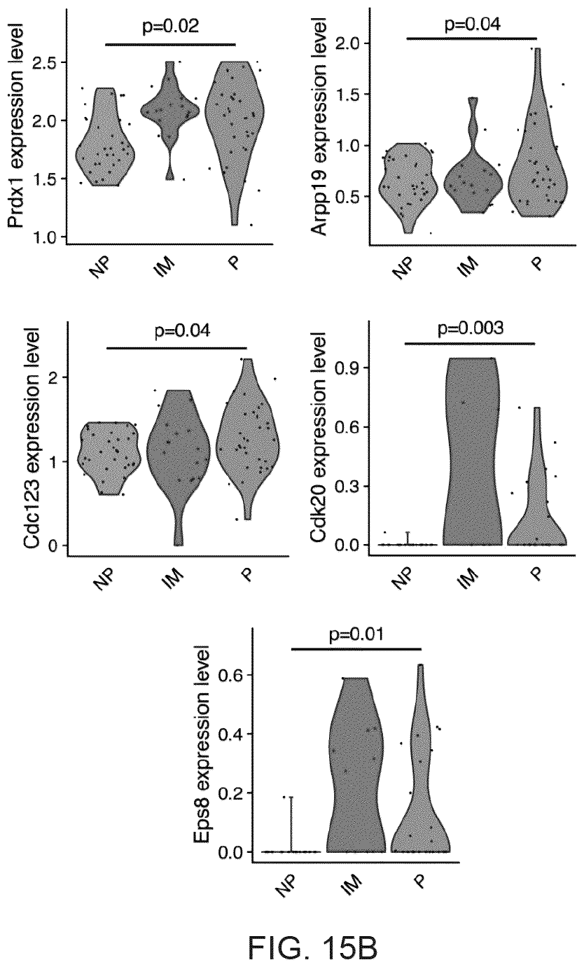

Next, early leukemia progression in a Hoxa9/Meis1-Ubiquitin-c-GFP (HA9M1) mouse model of AML was evaluated by performing intravital imaging of the calvarium bone marrow between 1 and 3 days after transplanting 3×106 cells into non-irradiated recipients (FIG. 14A). Starting from day 3, the number of leukemia cells per bone marrow cavity (concave endosteum) rapidly increases (FIG. 14B). This timepoint was chosen to study the mechanisms of early leukemia expansion. Longitudinal imaging of the same cavities in the same animals was used to quantify proliferation dynamics (see FIG. 14E for examples of longitudinal images) and classify cells as either proliferating (P, average of 30 cells per cavity on day 3), intermediate (IM, average of 13 cells per cavity on day 3) or non-proliferating (NP, average of 2 cells per cavity on day 3), as shown in FIG. 14C, where the median and quartile of the distributions are given. The 99.9% confidence interval of AML cells per cavity on day 3 was used to identify sites for cell extraction and sequencing. Prior to the sequencing experiments label dilution was quantified on day 3 after transplantation with CellTracker-labeled AML cells. The percentage of CellTracker-positive cells per bone marrow cavity decreased as the overall number of cells increased (FIG. 15A), further verifying the imaging-based classification. Surprisingly, these proliferative phenotypes were distributed throughout the calvarium bone marrow in a spatially heterogeneous manner (FIG. 14D), further underlining the need for a spatially resolved analysis by scRNA-seq.

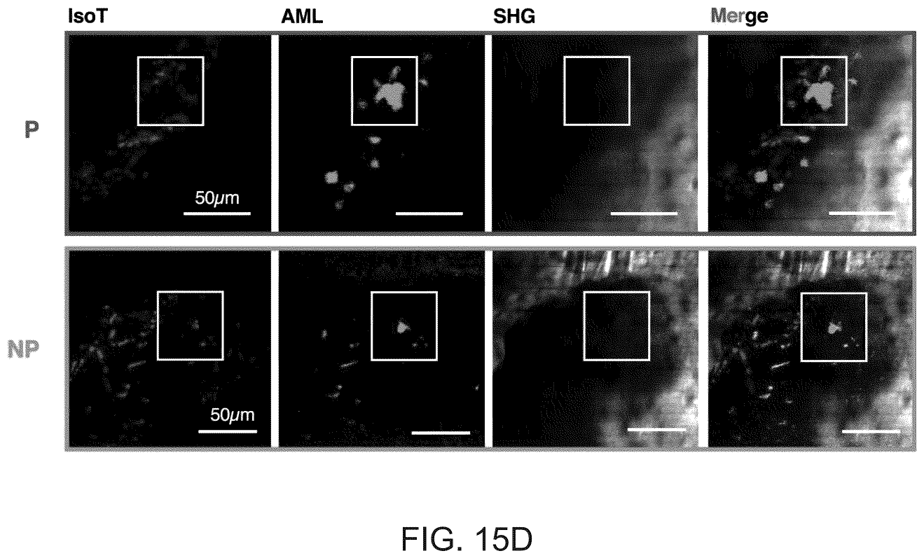

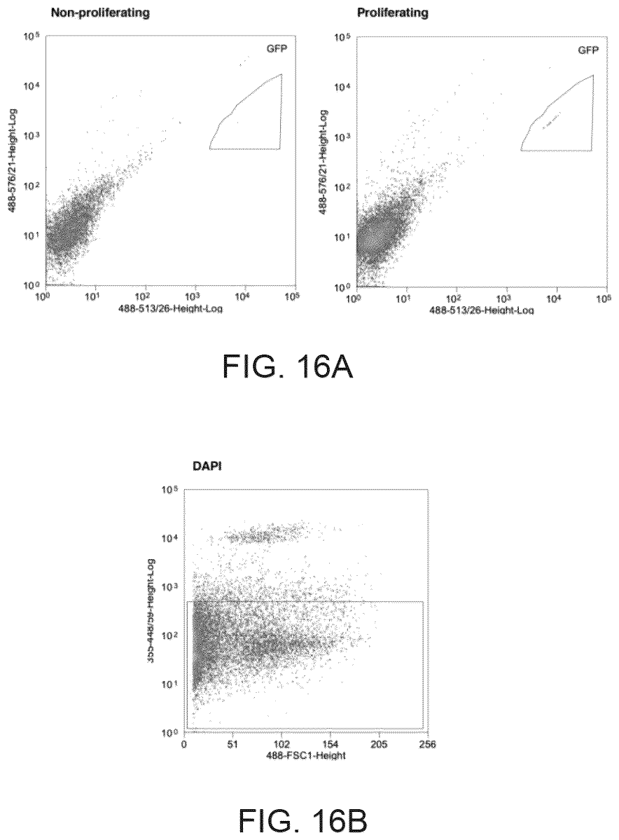

P, NP and IM AML cells were identified by imaging and aspirated by micropipette using the Image-seq platform. Subsequently, the AML cells were separated from the ˜100-400 surrounding hematopoietic cells by sorting them into individual wells of a 96-well plate by flow cytometry and gating for GFP (see FIGS. 16A-16B for examples of the gating strategy used to isolate an NP, as well as several P cells, from a single micropipette sample). By choosing this strategy (shown schematically in FIG. 10A as steps 1, 2, 3, 4 and 5b), the yield of the rare AML cells was optimized and the ˜30-40% cell loss that occurs during cell encapsulation using the 10× Genomics platform was avoided. In total, ˜40 cells were collected from four different locations in the P category, ˜40 cells from ˜30 different locations in the NP category, as well as ˜20 cells from three different locations in the IM category from n=11 mice. Experimental throughput was limited by the extremely rare NP cells, which made up only 0.7% of AML cells on day 3, with the overall leukemic burden already <0.01%. To minimize the issue of dropout that is often observed in scRNA-seq data, the SMARTseq-v4 protocol was used for reverse transcription and library construction. Because of the SMARTseq protocol's unique chemistry, the number of genes and transcripts detected per cell are higher than for any other scRNA-seq technology (FIG. 12D). Used in combination with the SMARTseq-v4 protocol, Image-seq therefore has the highest sensitivity and transcript coverage of any currently available spatial sequencing technology, with an average of 3,314,087 total mapped reads or 8,053 genes per cell. Of the 97 cells that were sequenced, a total of 84 cells passed quality control.

After sequencing and read alignment, cells that were in the G0 phase were identified and hierarchical clustering was performed based on cell cycle genes, identifying three separate clusters (shown in the heatmap of FIG. 15C). Cluster 1 was enriched in genes related to the S and G1/S phases, cluster 3 was enriched in genes related to the G2 and G2/M phases, and cluster 2 lacked expression of cycle genes and was therefore classified as quiescent cells (G0 phase). Interestingly, the distribution of cell cycle in the P and IM populations was very similar, with the largest proportion of cells in the G1/S and S phases, and the second largest proportion in G2 and G2/M (FIG. 14F). For the NP population, however, the major contribution came from the G0 phase, and the second largest from the G1/S and S phases. These results highlight the validity of the imaging-based classification, as well as the technology's ability to distinguish subtle phenotypic differences through imaging.

Next, AML cells were sub-clustered after regressing out cell cycle genes using Seurat, with the resulting UMAP embedding shown in FIG. 14H. Three major clusters were observed, and the heatmap in FIG. 14I shows the top 10 differentially expressed genes in each of them. The dark-green cluster (AML-mono) was enriched in progenitor cell markers of the monocytic lineage such as Ctss, Cnn3, Ms4a6c, Pira2 and Lsp1, as well as Itgb7 and Flt3 (FIG. 14I), which have been associated with stronger leukemia-initiating capacity in Hoxa9/Meis1 AML. AML-GMP expressed granulocyte-monocyte progenitor markers such as Mpo, Elane, Prss57 and Ctsg, along with Gstm1, which has been associated with quiescent, therapy-resistant, leukemia-initiating cells in AML. AML-AP1 expressed Prtn3, which is a marker of the monocytic lineage, along with Jun and Fos, which comprise the AP1 transcription complex, as well as Dusp1, which are all part of the MAP-kinase signaling pathway and are associated with cell proliferation. Cluster AML-GMP consisted primarily of cells that were classified as NP by imaging, AML-AP1 consisted primarily of P cells, and AML-mono consisted primarily of P and IM cells (a detailed breakdown is given in G 14G).

Increased proliferation of DPP4-positive cells in mouse and human AML

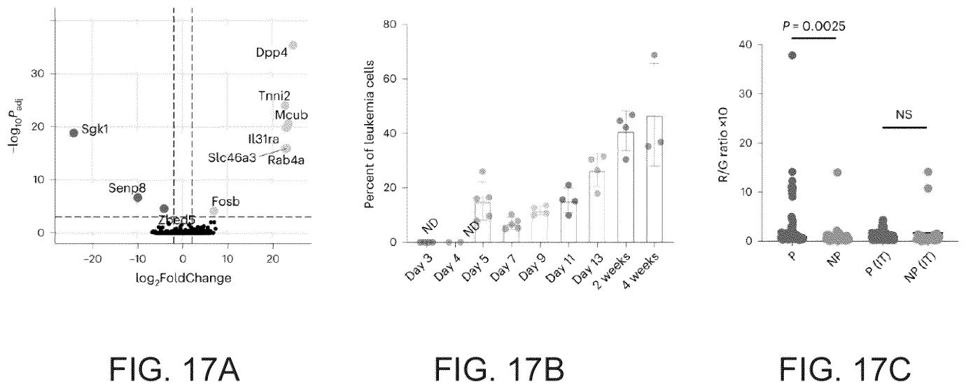

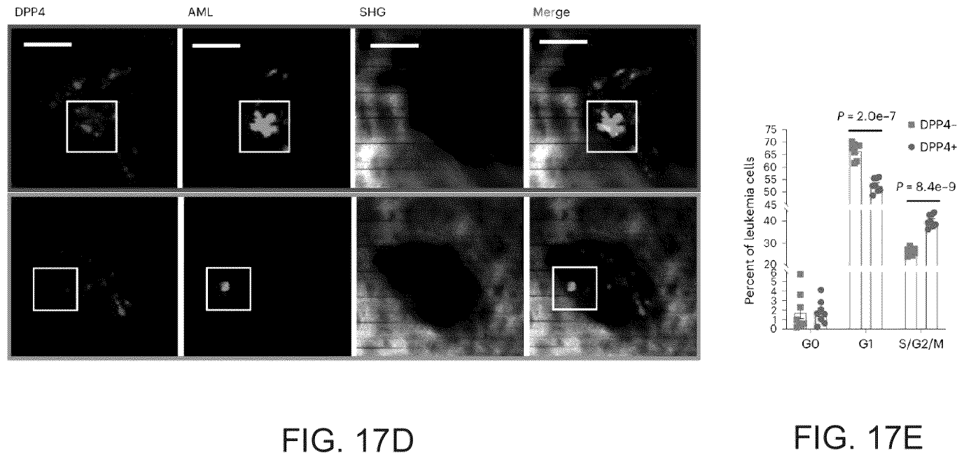

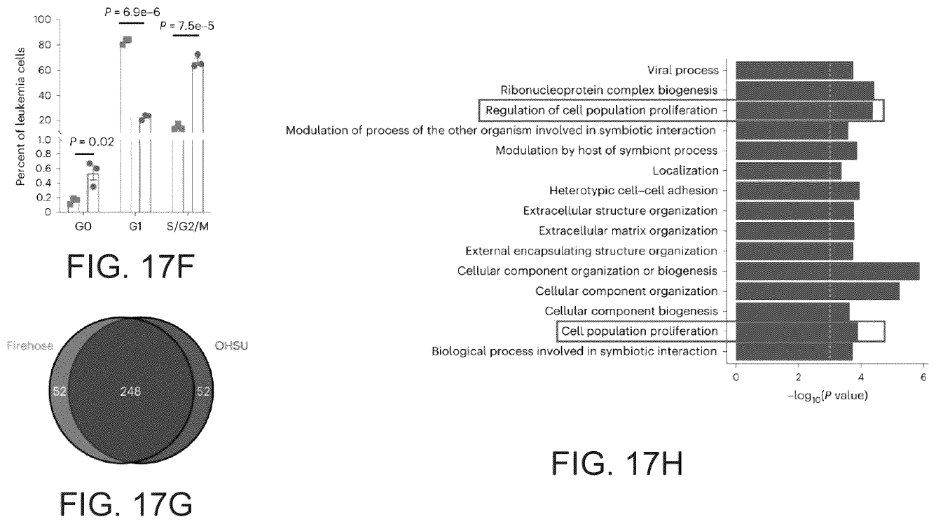

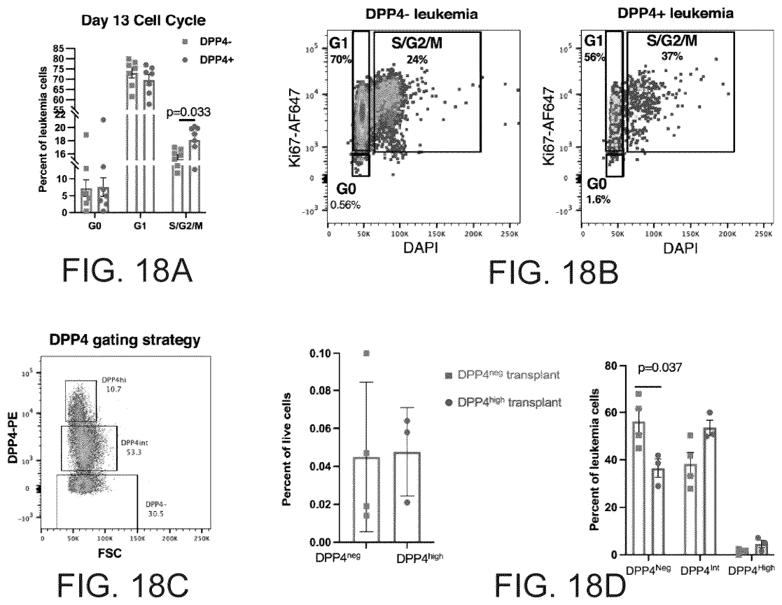

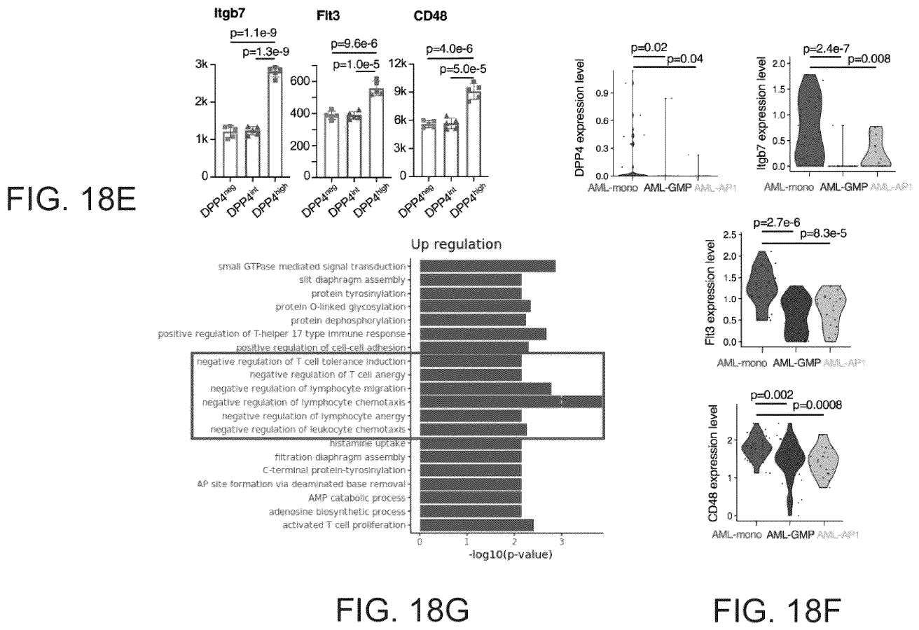

With the goal of identifying signals that trigger the exit of leukemia-initiating cells from the quiescent state, the differential expression of genes between P and NP cells was investigated (FIG. 17A) without prior regression of cell cycle genes. GO analysis identified several terms related to the negative regulation of immune response (FIG. 18G). The most strongly differentially expressed gene, both when comparing P with NP and the entire population of P+IM with NP cells, was Dpp4. DPP4 or CD26 is a transmembrane protein that functions as a serine protease, selectively cleaving the amino-terminal, penultimate proline, or alanine of proteins. Among its many substrates are granulocyte-macrophage colony-stimulating factor, CXCL12, granulocyte colony-stimulating factor, interleukin 3 and erythropoietin, which are known to enhance proliferation, survival, chemotaxis, homing and engraftment of hematopoietic stem and progenitor cells. DPP4 has also been shown to cleave CCL2, CCL3, CXCL6, CXCL9 and CXCL10, which play a role in decreasing the survival and proliferation of hematopoietic stem and progenitor cells. Several studies have implicated DPP4 as a leukemic stem cell marker in chronic myeloid leukemia, and recent studies of human AML have linked increased DPP4 expression to a poorer overall survival and have associated AML-derived DPP4 with the suppression of normal hematopoietic progenitor cell proliferation. However, DPP4 was previously not known to play a role in early AML proliferation.

To validate DPP4 expression its protein expression was assessed using flow cytometry and found that it increases as a function of disease progression, with a maximum stable expression at ˜50% by week 4 (FIG. 17B). Although DPP4 expression was too low to be detected using flow cytometry on day 3, it could be detected using in vivo imaging, a statistically significant difference was found when DPP4 antibody labeling was compared in P versus NP cells whereas this was not observed in the corresponding isotype control (FIGS. 17C-D and FIG. 15D). Next, the cell cycle in DPP4-positive versus DPP4-negative AML cells was analyzed using Ki-67 staining (FIG. 18B for gating strategy), which confirmed that DPP4-positive cells have a higher cycling rate (FIG. 17E and FIG. 18A). To elucidate whether this phenotype is more broadly observed, an MLL-AF9 model of AML was studied and found the decrease in the G1 phase and the concomitant increase in the S, G2 and M phases in DPP4-positive cells to be even more pronounced (FIG. 17F). Note that at day 13 (FIG. 18A) the difference in cycling rate is less pronounced than at day 7, and there is no difference in the proportion of cells in the G1 phase. This is a very aggressive model and even at day 13 more than 80% of the marrow is composed of leukemia cells, suggesting that the cells are running out of space to proliferate, which is likely to reduce the cycling rate. In keeping with this, the overall proportion of DPP4-positive and DPP4-negative cells that are actively cycling is considerably lower on day 13 than on day 7 (P<0.0001), which is likely the reason that the proportion of DPP4-positive cells reaches a maximum stable value of 50%. Additionally, DPP4-correlated genes were analyzed in two published, human AML datasets and observed a high overlap between the top 300 positively correlated genes. GO analysis of the overlapping gene set (FIG. 17G) showed an enrichment of terms related to cell cycle (FIG. 17H), suggesting that the phenotype is relevant to human AML.

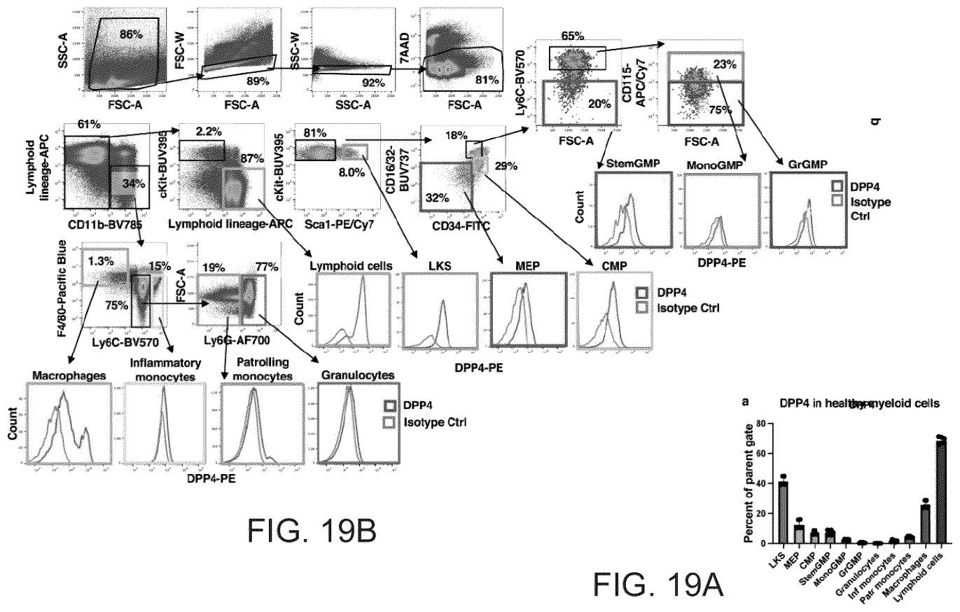

Because DPP4 expression was confined mainly to the AML-mono cluster, which expressed high levels of Itgb7, Flt3 and Cd48 (FIG. 18F), their cell surface expression was analyzed using flow cytometry. A strong increase in Itgb7 expression was found, as well as a moderate increase in Flt3 and CD48 for cells that had high DPP4 expression as compared with negative and intermediate DPP4 expression (FIG. 18C and FIG. 18E), suggesting that this combination of cell surface markers can be used to identify cells belonging to AML-mono. Interestingly, overexpression of Flt3 has been associated with poor prognosis in AML, and combined expression of Itgb7 and Flt3 has been used to identify leukemic stem cells in Hoxa9/Meis1 AML. DPP4 expression in healthy myeloid precursors was investigated, as well as lineage-specified cells, using flow cytometry (FIG. 19A). Higher DPP4 levels were found on cells that are more immature (with the exception of macrophages and lymphoid cells), which supports the notion that DPP4 expression is not due to progression along the myeloid differentiation trajectory in AML cells, or associated with priming towards a specific lineage.

To assess leukemia-initiating capacity, 1,000 DPP4high and DPP4neg AML cells were isolated 3 weeks after transplantation using flow cytometry. They were transplanted into fresh, non-irradiated recipients and compared leukemic burden and the proportion of DPP4-positive and DPP4-negative cells at day 10 (FIG. 18D). Interestingly, a subset of DPP4-negative cells converted to being DPP4 positive and vice versa, suggesting that DPP4 expression could be induced by the microenvironment.

Activation of DPP4 Expression by the Microenvironment

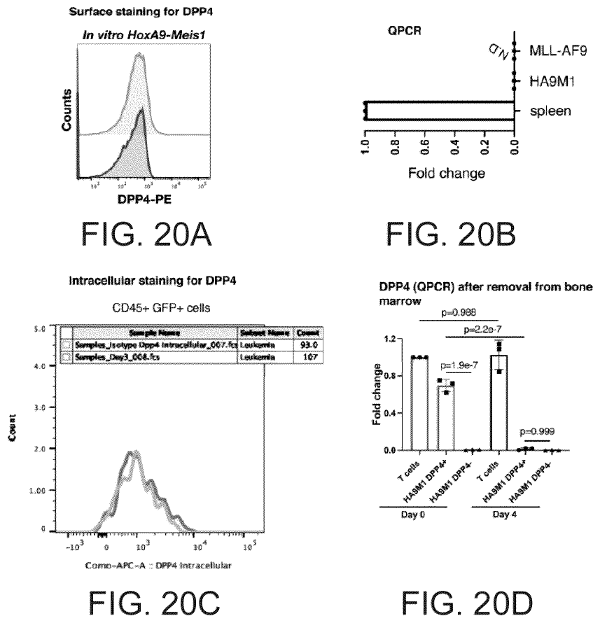

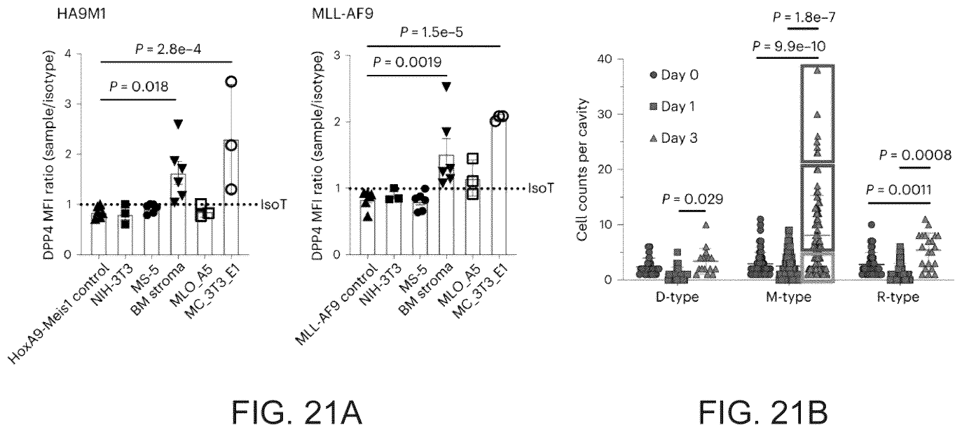

While observing DPP4 cell surface expression to be highly stable and reproducible in vivo, no DPP4 on AML cells cultured in vitro were detected (FIG. 20A). DPP4 by intracellular staining or quantitative polymerase chain reaction (qPCR) was also not detected (FIG. 20B and FIG. 20C). In addition, although DPP4-positive HA9M1 cells expressed Dpp4 at comparable levels to splenic T cells immediately following removal from the bone marrow environment (day 0 qPCR results in FIG. 18D), they subsequently lost all Dpp4 expression at the transcriptional level after 4 days of in vitro cell culture. Remarkably, co-culture of AML cells with bone marrow stromal and osteoblast precursor cells activated DPP4 cell surface expression, while co-culture with other stromal cell lines did not (FIG. 21A). Combined with the observation that DPP4 expression is upregulated in specific bone marrow compartments (FIGS. 17A and 17C), the data suggest that different niches drive the upregulation of DPP4. Given the recent finding that hematopoietic stem cell proliferation is restricted to a subset of bone marrow cavities with mixed bone remodeling activity (M-type cavities) it was reasoned that these niches could drive proliferation of AML cells. Dye labeling was used to distinguish deposition (D-type), resorption (R-type) and mixed (M-type) bone marrow cavities by in vivo imaging, finding that P cells are exclusively located in M-type cavities (FIG. 21B).

### Discussion

AML is an aggressive blood cancer characterized by an accumulation of immature myeloid cells in the bone marrow that are arrested in differentiation and that accumulate as immature and malignant self-renewing progenitors. Despite an initially favorable response to treatment with intensive cytotoxic chemotherapy, ˜75% of patients die within 5 years of diagnosis. Relapse is thought to be driven by the rare pool of leukemia-initiating cells that persist in the bone marrow following chemotherapy. An important approach to the development of new therapeutics is the targeting and exploiting of the supportive interaction as leukemia-initiating cells communicate with and seek refuge within the bone marrow microenvironment. Studying and identifying signals that trigger the exit of leukemia-initiating cells from the quiescent state thereby provides mechanistic insight into disease recurrence. Examining the bone marrow at a stage of very low leukemic burden, comparable to the state of minimal residual disease (when the AML cells are more likely to be surrounded by normal hematopoietic and stromal cells), provides unique insights that cannot be obtained using traditional assays of relatively high leukemic burdens in which the bone marrow is crowded with malignant cells and the microenvironment is severely altered.

The above results show how the combination of intravital microscopy to study the dynamics of AML disease progression, and spatially resolved scRNA-seq, provides new insights into leukemia biology. Moreover, multiphoton microscopy uniquely informs the 3D spatial context of AML cells and can be used to validate the single-cell gene expression data in a spatially resolved manner (FIG. 17C and FIGS. 17D and FIG. 15D), as well as characterize different microenvironments (FIGS. 14B-E and 21B) that can be analyzed for their differential expression (FIG. 17A). It was found that DPP4 marks a more proliferative phenotype both in murine and in human disease, which makes it a promising target for further investigations that could lead to new treatments.

In situ imaging of murine bone marrow has led to numerous insights into the basic biology of hematopoietic stem cells, as well as the spatial organization of bone marrow tissue. In the skull bone marrow, intravital microscopy has been used for the study of hematopoiesis, along with hematopoietic stem cell and leukemia biology. It has elucidated the temporal dynamics of these processes and tracked the association of individual cells with distinct bone marrow components in real time. Imaging alone, however, fails to provide unbiased mechanistic insight into the observed cellular dynamics and spatial organization. It is precisely such information, however, that promises to bring new insights into hematopoietic and leukemia biology and, concomitantly, the development of new therapeutics.

To date, the only spatially resolved transcriptional study of the murine bone marrow, a tissue that remains highly challenging to section, relied on bulk transcriptomic analysis of tissue blocks that were cut from formalin-fixed bone marrow sections using laser micro-dissection. The cellular composition of individual blocks was then inferred computationally using a separate, scRNA-seq dataset. At present there have been no spatially resolved transcriptional studies of the leukemic bone marrow.

Image-seq represents a new experimental approach for integrating spatial and molecular information. Multiple contrast mechanisms can be used to visualize the procedure and reconstruct the 3D spatial position of the extracted cell sample, including autofluorescence, confocal reflectance (FIGS. 6A-6B) or labeling with a fluorescent membrane dye such as Di-8-ANEPPS (used in the experiments in FIGS. 7D and 10A-10H). When combined with intravital microscopy, Image-seq additionally enables transcriptomic analysis on cells for which the position and history have been documented by intravital microscopy. Because of its utility both for high-throughput sequencing and in-depth profiling of rare cells, the Image-seq platform is highly versatile in its applications. Additionally, it is expected that it can be applied to study fixed tissues and tissue sections (for example, using the 10× Genomics protocol for fixed tissues instead of the protocol detailed here), although working with live cell samples is recommended when possible to achieve the highest sensitivities and transcript coverage. Currently there is a trade-off between spatial resolution and throughput. Although it is possible to isolate very few cells and obtain a spatial resolution of ˜30×30×30 μm, or even a single cell if there is only one fluorescent cell in the isolated tissue volume (FIGS. 6A-6B) that can be sorted by flow cytometry, this comes at the cost of lowered throughput. Increasing the number of extracted cells, however, lowers spatial resolution. The Image-seq method and systems will be used to investigate a range of diseases and biological questions involving the bone marrow, along with other tissues that remain difficult to section. Furthermore, based on the technology's ability to isolate viable cells, its applicability to spatially resolved multi-omics studies in a range of biological settings is anticipated.

### Methods

Microscope

Intravital microscopy and plasma-mediated laser ablation were performed using a custom-built multiphoton microscope. The output of a femtosecond, 1,550 nm fiber laser (Calmar Cazadero) operating at a repetition rate of 5 MHz was split into two optical paths: one was frequency doubled with a BiBO crystal (Newlight Photonics) to obtain a wavelength of 775 nm that was used for either imaging or ablation (FIG. 3B). The other was coupled into a large mode-area photonic crystal fiber (LMA-35, NKT Photonics) where, by soliton self-frequency shift, radiation with a frequency tunable between 1,880 nm and 1,960 nm was generated. This was frequency doubled (BiBO, Newlight Photonics) to obtain imaging wavelengths between 940 and 980 nm. The imaging and ablation beams were spatially overlapped using a dichroic mirror (Zt 785 RDC, Chroma) and directed to the scanning optics, which consisted of a revolving polygon (36 facets, 14,400 r.p.m., x scan) conjugated to a scanning galvanometer mirror (15 Hz, y scan), and which were conjugated to the back aperture of the objective lens (Olympus, ×60, numerical aperture (NA) 1.0, water immersion). This served to steer the imaging and ablation beams across the microscope field of view. Typical pulse energies used for imaging were between 1 and 2 nJ and typical pulse energies for ablation were between 10 and 15 nJ. For stable plasma generation a continuous stream of PBS (ThermoFisher Scientific) was flushed across the sample at a rate of 10 ml min⁻¹ to remove any gas or debris generated during the ablation procedure. The sample (mouse) was positioned in the image plane using a micromanipulator (Sutter instruments) and translated along the z dimension to image or ablate a 3D volume of tissue. Fluorescence signals were collected after excitation with 775 nm or 980 nm laser light, using three photomultiplier tubes (PMTs). The typical configuration of the dichroic mirrors was FF705 LP, FF495 LP and FF552 LP, with filters 439/154 (blue PMT R7600-U300, Hamamatsu) for the detection of collagen second-harmonic generation signal from bone, 525/50 (green PMT R7600-U300, Hamamatsu) for the detection of AML cells and tetracycline, and 607/70 (red PMT R7600-U20, Hamamatsu) for the detection of CellTracker Red and Di-8-ANEPPS (ThermoFisher Scientific) signals. An avalanche photodiode was also installed for collection of the confocal reflectance signal.

Procedure for Intravital Imaging

The procedure for intravital imaging of the calvarium bone marrow is described in detail elsewhere 61. Prior to intravital microscopy and ablation, analgesics were given (buprenorphine at 0.05-0.1 mg kg⁻¹ i.p.), mice were anesthetized using vaporized isoflurane (3-4% for induction, 1-2% for maintenance) and depth of anesthesia ensured by toe pinch. Hair around the incision site on the scalp was trimmed, and skin was made aseptic using a betadine scrub. The incision (˜5 mm×7 mm) was made using sterile surgical scissors, and the skin folded back to expose the skull bone, which was hydrated using sterile PBS. Mice were transferred to a mouse holder with integrated heating pad (37° C.), and a continuous stream of isoflurane supplied via a nose cone during in vivo imaging and ablation. Intravital microscopy experiments were carried out using the microscope and dichroic mirror-filter configuration detailed above, and image stacks were acquired with a 2 μm step size from the calvaria surface and by averaging 15 frames to obtain a single image. At the end of each imaging session the mice were either sacrificed or survival surgery was performed. For survival, the exposed skull was extensively irrigated with sterile saline and the scalp closed with surgical sutures (Ethicon 6-0 nylon monofilament, Ethicon). After closure, 0.25% bupivacaine (2 mg per kg animal weight) was administered to the surgical site via percutaneous infiltration to aid with pain management, and triple antibiotic ointment (Curad) was applied on the sutured area. The animal was returned to its cage and monitored until awake. Buprenorphine (0.05-0.1 mg per kg animal weight) was given i.p. or s.c. along with topical antibiotic ointment every 8-12 h for up to 2 days after surgery.

In Vivo Cell Aspiration

The site for cell extraction was identified by intravital imaging of the calvarium bone marrow (procedure detailed above). A volume of bone ˜40 μm×200 μm×300 μm was removed using laser ablation (pulse energy 14 nJ). A circular channel was etched (diameter ˜100 μm, depth 20-30 μm, pulse energy 10 nJ) by placing an iris in the intermediate image plane, and a micropipette (MPB-FP-20, Origio) was inserted through the channel and into the bone marrow using a micromanipulator (Sutter Instruments). The target cell was aspirated by suction with an Air Syringe (Cooper Surgical) and the procedure was visualized using a combination of multiphoton and confocal reflectance signals. The ablation procedure itself was performed at a rate of 0.25 μm per 670 ms along the z dimension, which corresponded to 10 passes per plane using the 15 frame per second imaging rate of the optical system.

Image-Guided Cell Isolation for Flow Cytometry and Sequencing

In Vivo Imaging of Calvarium

Prior to cell isolation the mice underwent intravital imaging, the sites for cell extraction were identified and their spatial position recorded with respect to the bregma and lambda reference points. Mice were retro-orbitally injected with either Di-8-ANEPPS (1.9 mg kg⁻¹) or Brilliant Violet 421 anti-mouse CD31 antibody (1 mg kg⁻¹, BioLegend) to aid in the visualization of the cell isolation procedure. Anesthesia was increased to 4%, the mice were transferred to a dissection tray and were transcardially perfused as follows:
a. For the isolation of hematopoietic cells: first with an ice-cold solution of 5 μM EDTA in PBS (ThermoFisher Scientific, flow rate 5 ml min⁻¹, total volume 10 ml) and then with ice-cold PBS (ThermoFisher Scientific, no Ca or Mg, flow rate 5 ml min⁻¹, total volume 10 ml).
b. For the isolation of stromal cells: first with a 37° C. solution of 5 μM EDTA in PBS (both ThermoFisher Scientific) at a flow rate of 5 ml min⁻¹ and with a total volume of 10 ml, and then with an enzymatic digestion buffer at 37° C. (flow rate 5 ml min⁻¹, total volume 10 ml). The mice were then incubated at 37° C. for 20 min. The enzymatic digestion buffer consisted of 450 U ml⁻¹ Collagenase I (Sigma), 125 U ml⁻¹ Collagenase XI (Sigma), 60 U ml⁻¹ Hyaluronidase (Sigma) and 60 U ml⁻¹ DNase I (Sigma) in 20 ml Medium-199 (Gibco).

Mice were transferred back to the microscope and samples were sequentially isolated from positions marked for cell extraction.

In Situ Imaging of Tibia

Mice were perfused first with an ice-cold solution of 5 μM EDTA in PBS (ThermoFisher Scientific, flow rate 5 ml min⁻¹, total volume 10 ml) and then with ice-cold PBS (ThermoFisher Scientific, no Ca or Mg, flow rate 5 ml min⁻¹, total volume 10 ml). Tibia were then dissected and cleaned, and the tibial bone was thinned to a thickness of ˜50 μm using a razor blade. Bones were mounted onto a microscope slide by fastening a piece of modeling clay to the glass slide and gently pressing the bone onto the modeling clay. The mounted bone was then transferred to the microscope for in situ imaging.

Image-Guided Cell Aspiration (Calvarium and Tibia)

In each location the procedure was as follows: first, a volume of bone ˜40 μm×200 μm×300 μm was removed using a pulse energy of 14 nJ; second, a channel with dimensions ˜30 μm×50 μm×100 μm was created using a pulse energy of 10 nJ; third, the micropipette was inserted through this channel and positioned next to the target cells; and last, the cells were aspirated using a micropipette (MBB-FP-M-20, Origio) and transferred to an Eppendorf tube filled with 5 μl Medium-199 with 2% v/v FBS. Samples were kept on ice until they were either analyzed using flow cytometry (validation experiments), transferred to the 10× Chromium platform, or sorted into individual wells of a 96-well plate by flow cytometry (for library preparation by SMARTseq-v4). The ablation procedure itself was performed at a rate of 0.25 μm per 670 ms along the z dimension, which corresponded to 10 passes per plane using the 15 frame per second imaging rate of the optical system.

Prior to the experiment, micropipettes were coated with Sigmacote (flowed through the micropipette for 2 min at a rate of 200 μl min⁻¹) to prevent cells from adhering to the glass surface, as well as with Qtracker 655 vascular labels (5 μl were pipetted up and down several times) to fluorescently coat the pipette and aid with visualization.

Collection of Whole Bone Marrow Preparations

Calvaria were dissected and cut into smaller pieces. Tibia were dissected and cleaned. To aid in the release of the bone marrow, calvaria bone fragments or whole tibia bones were gently crushed in Medium-199 (Gibco) supplemented with 2% FBS (Gibco). The resulting cell suspension was subsequently passed over a 70 μm cell strainer (BD Falcon).

Cell Lines

Syngeneic Leukemia Model

In brief, the HoxA9/Meis1 AML cell line was generated by retroviral transduction with an MSCV-HoxA9-IRES-Meis1 construct (originally designed by G. Sauvageau) into bone marrow mononuclear cells from a mouse expressing GFP under the control of the ubiquitin, and luciferase under the control of the β-actin promoter. The MLL-AF9 cell line used for the cell cycle experiments was generated by collecting bone marrow from a 5-fluorouracil-treated Cas9-GFP mouse, followed by two consecutive transfections with retroviral MLL-AF9. For both models the cells were transplanted into irradiated recipients, collected from terminally ill animals and re-transplanted into a second set of irradiated recipients, from which GFP-expressing cells were collected close to the disease endpoint. These cells were cultured in RPMI 1640 (Gibco) supplemented with 10% FBS (ThermoFisher Scientific), 100 IU ml⁻¹ penicillin (Corning), 100 mg ml⁻¹ streptomycin (Corning), 5 ng ml⁻¹ interleukin 3 (IL-3, Peprotech), as well as 100 ng ml⁻¹ stem cell factor (SCF, Peprotech) for the HoxA9/Meis1, and 20 ng ml⁻¹ SCF and 10 ng ml⁻¹ IL-6 (both from Peprotech) for the MLL-AF9. Recipient female mice (10-12 weeks old) were injected with 3×106 cells in 200 μl PBS (HoxA9/Meis1) and 1×106 cells in 200 μl PBS (MLL-AF9).

The MLL-AF9 model used for the experiments in FIG. 3B was generated by crossing MLL-AF9 knock-in mice64 with mice expressing GFP under the control of the ubiquitin, and luciferase under the control of the β-actin promoter. The bone marrow from a terminally ill male mouse was collected and cultured in vitro using RPMI 1640 (Gibco) supplemented with 10% FBS (Gibco), 100 IU ml⁻¹ penicillin, 100 mg ml⁻¹ streptomycin (both from Corning), 20 ng ml⁻¹ recombinant mouse SCF (rmSCF), 10 ng ml⁻¹ recombinant mouse IL-3 (rmlL-3) and 10 ng ml⁻¹ rmIL-6 (all from R&D Systems). Recipient male mice (8-10 weeks old) were injected with 1×106 cells in 100 μl saline.

For transplantation of DPP4-negative and DPP4-positive HoxA9/Meis1 leukemia, cells were isolated from the long bones and vertebral column 3 weeks after transplantation. The marrow underwent density gradient centrifugation (Ficoll-Paque Plus, Cytiva Life Sciences) at 400×g for 25 min at room temperature with no brake. The mononuclear layer was isolated and subsequently blocked in PBS with 2% FBS and murine Fc Block (BD Biosciences, dilution 1:50). Following this, the samples were stained with CD45-APC/Cy7 (BD Biosciences, dilution 1:100) and DPP4-PE (Biolegend, dilution 1:20). To exclude dead cells, samples were incubated with 7-aminoactinmycin D (7AAD, 0.25 μg, BD Biosciences) and then the 7AAD−GFP+CD45+DPP4− and 7AAD−GFP+CD45+DPP4+ cells were sorted into separate tubes. A total of 1,000 cells of each phenotype were injected into 10-12-week-old recipient mice. Leukemia burden and DPP4 expression were assessed 10 days post-transplantation.

Simian Virus 40 Immortalized Bone Marrow Stroma

Total bone marrow cells were isolated from the femurs and tibias of B6J.129(B6N)-Gt(ROSA)26Sortm1(CAG-cas9*,−EGFP)Fezh/J mice (Jackson Laboratories). The bones were crushed in PBS (ThermoFisher) with 2% FBS (Gibco) and the released marrow was filtered over a 40 μm strainer. Mononuclear cells were collected by density gradient centrifugation (Ficoll-Paque Plus, Cytiva Life Sciences). A total of 20×106 mononuclear cells were put into culture with Alpha-MEM (Gibco) supplemented with 20% FBS (Gibco) and 1% Penicillin-Streptomycin (Gibco) in 150 mm dishes. Non-adherent cells were discarded around day 5 and the media changed every 5-7 days for approximately 3 weeks. At this point, colonies of large adherent fibroblasts were apparent. The cells were detached from the dishes with Trypsin-EDTA (Gibco), counted, and 50,000 cells seeded into two wells of a six-well plate. The following day, one well of cells was transduced with lentivirus in the presence of 8 μg ml⁻¹ polybrene (Millipore, Sigma) to deliver the simian virus 40 (SV40) small and large T antigen. The plasmid pLenti CMV/TO SV40 small+Large T (w612-1) was a gift from Eric Campeau (Addgene plasmid 22298; http://n2t.net/addgene:22298; RRID: Addgene_22298). Despite the lack of a selectable marker, the transduced cells divided much more rapidly than the non-transduced primary stroma, and over the course of 2-3 passages, established an SV40 immortalized stromal cell line.

Isolation and In Vitro Stimulation of T Cells

Spleens were collected from C57B1/6 J mice and a single-cell suspension was obtained by mechanical dissociation of the tissue over a 70 μm cell strainer (BD Falcon) in RPMI 1640 (ThermoFisher) supplemented with 10% FBS (Gibco) and 1% Penicillin-Streptomycin (Gibco). Red cells were lysed using ACK Lysing Buffer (Quality Biological) followed by removal of non-T-cell splenocytes through magnetic depletion. In brief, the cell suspension was adjusted to 108 cells ml⁻¹ and incubated with biotinylated antibodies directed against B220, CD19, Ter119, NK1.1, Cd11b and Gr1 (all from Biolegend, dilution of 1:100) at a concentration of 5 μg ml⁻¹ for 10 min at room temperature on an orbital shaker. This was followed by the addition of 25 μl ml⁻¹ streptavidin-conjugated Rapidspheres (Stem Cell Technologies) and an additional 5 min of incubation at room temperature on an orbital shaker. The samples were placed in an EasySep magnet (Stem Cell Technologies) for 5 min and the purified T cells were subsequently decanted into a fresh tube. Purity of the isolated T cells was confirmed to be >95% using FACS analysis. To induce T-cell proliferation, the isolated splenic T cells were plated with the T-cell activator Dynabeads CD3/CD28 (ThermoFisher) in a 1:1 ratio in leukemia cell line medium:RPMI 1640 (Gibco) supplemented with 10% FBS (Gibco), 100 IU ml⁻¹ penicillin (Gibco), 100 mg ml⁻¹ streptomycin (Gibco) and 5 ng ml⁻¹ IL-3 (Peprotech), as well as 100 ng ml⁻¹ SCF (Peprotech).

Flow Cytometry

Whole Bone Marrow and Micropipette Samples

Whole bone marrow and micropipette samples from calvarium and tibia were blocked with anti-mouse Fc Block (BD Biosciences, dilution 1:50) for 10 min at 4° C. The cells were thereafter stained with a blood cell lineage cocktail for 30 min at 4° C. For detection of dead cells 7AAD (BD Biosciences, 0.25 μg) was added to the sample prior to analysis. Flow cytometry was performed on a BD FACS Aria III sorter (BD Biosciences) and all data were analyzed using FlowJo (Treestar).

Flow Sorting of AML Cells for SMARTseq-v4

Prior to sorting, 1 ml PBS (ThermoFisher Scientific) was added to each sample tube, along with 0.1 μg DAPI (ThermoFisher Scientific). The sample was incubated for 10 min, gently vortexed and transferred to the flow cytometer (MoFlo Astrios EQ cell sorter). Single, live, GFP-positive AML cells (see FIGS. 16A-16B for examples of gating strategy) were sorted into individual wells of a 96-well PCR plate filled with 2.6 μl Lysis Buffer (Takara Bio). Plates were sealed, spun down, snap-frozen and stored at −80° C. prior to preparation for complementary DNA synthesis using the SMARTseq-v4 assay.

Flow Analysis and Sorting of CXCL12+ Stromal Cells for SMARTseq-v4

Prior to sorting or analysis, samples were cell-surface stained with anti-CD45-BV421 (BioLegend, dilution 1:100) for 30 min at 4° C. in Medium-199 (Gibco) supplemented with 2% FBS. Prior to sorting, 1 ml PBS (ThermoFisher Scientific) was added to each sample tube, along with 0.1 μg DAPI (ThermoFisher Scientific). The sample was incubated for 10 min, gently vortexed and transferred to the flow cytometer (MoFlo Astrios EQ cell sorter). Flow cytometry data were analyzed using FlowJo (Treestar). Single, live, CD45−DsRed+stromal cells (see description of gating strategy disclosed herein) were sorted into individual wells of a 96-well PCR plate filled with 2.6 μl Lysis Buffer (Takara Bio). Plates were sealed, spun down, snap-frozen and stored at −80° C. prior to preparation for cDNA synthesis using the SMARTseq-v4 assay.

Leukemia Burden and DPP4

Leukemic bone marrow was blocked using anti-mouse Fc Block (BD Biosciences, dilution 1:50) for 10 min at 4° C. in Medium-199 supplemented with 2% FBS. Surface staining was thereafter performed with CD45-APC/Cy7 (BD Biosciences, dilution 1:100) and DPP4-PE (BioLegend, dilution 1:20) for 30 min at 4° C. The cells were then washed and resuspended in Medium-199 supplemented with 2% FBS with 0.25 μg 7AAD (BD Biosciences). Flow cytometry was performed on a BD FACS Aria III sorter (BD Biosciences) and all data were analyzed using FlowJo (Treestar). FIG. 18 shows the gating strategy used to distinguish DPP4high, DPP4int and DPP4neg cells. Note that DPP4-positive cells were defined as DPP4high and DPP4int.

Leukemia Cluster Cell Surface Markers

Leukemic bone marrow was blocked using anti-mouse Fc Block (BD Biosciences, dilution 1:50) for 10 min at 4° C. in Medium-199 supplemented with 2% FBS. Surface staining was thereafter performed with a leukemia cluster cocktail for 30 min at 4° C. The cells were then washed and resuspended in Medium-199 supplemented with 2% FBS with 0.25 μg 7AAD (BD Biosciences). Flow cytometry was performed on a BD FACS Aria III sorter (BD Biosciences) and all data were analyzed using FlowJo (Treestar).

Intracellular DPP4 Staining

Bone marrow from leukemia-bearing mice was incubated with anti-mouse Fc Block (BD Biosciences, dilution 1:50) for 10 min at 4° C. followed by surface staining with anti-CD45-APC/Cy7 (BD Biosciences, dilution 1:100). Samples were washed and stained with LIVE/DEAD fixable viability dye (ThermoFisher) in accordance with the manufacturer's instructions. The cells were thereafter fixed with Cytofix/Cytoperm (BD Biosciences) for 20 min at 4° C. 1×Perm/Wash buffer (BD Biosciences) was then used to wash the cells and the cells were incubated with either anti-DPP4 or an isotype control antibody (Biolegend, dilution 1:20 for both) that were both conjugated to Alexa Fluor 647 in-house (Abcam) for 30 min at room temperature. The cells were then washed one last time in Perm/Wash buffer (BD Biosciences) and resuspended in Medium-199 supplemented with 2% FBS for analysis. Flow cytometry was performed on an LSR II instrument (BD Biosciences) and all data were analyzed using FlowJo (Treestar).

Cell Cycle Analysis

Bone marrow isolated from leukemic mice was blocked with anti-mouse Fc Block (BD Biosciences, dilution 1:50) for 10 min at 4° C. Surface staining with anti-CD45-APC/Cy7 (dilution 1:100) and DPP4-PE (dilution 1:20) was performed at 4° C. for 30 min. Following this, the samples were washed in Medium-199 supplemented with 2% FBS and then fixed with Cytofix/Cytoperm (BD Biosciences) for 20 min at 4° C. The fixed cells were thereafter washed with 1×Perm/Wash buffer (BD Biosciences) and resuspended in Perm/Wash buffer containing anti-Ki67-AF647 at a 1:10 dilution for a 30 min incubation. The samples were washed one more time with 1×Perm/Wash buffer (BD Biosciences) and then incubated in 1×Perm/Wash buffer (BD Biosciences) with 2 μg ml⁻¹ DAPI (Biolegend) for 10 min. Finally, the samples were spun down to remove the DAPI-containing buffer and resuspended in Medium-199 supplemented with 2% FBS for analysis. Flow cytometry was performed on a BD FACS Aria III sorter (BD Biosciences) and all data were analyzed using FlowJo (Treestar).

Analysis of DPP4 Expression Following Co-Culture

A total of 250,000 MLL-AF9 or HoxA9-Meis1 leukemia cells were plated at a 1:1 ratio with the following stromal cell lines:NIH-3T3 (American Type Culture Collection, ATCC), MS-5 (RIKEN), MLO-A5 (Kerafast), MC-3T3-E1 (ATCC) and SV40 immortalized bone marrow stroma. The cells were grown in RPMI 1640 (Gibco) supplemented with 1% Penicillin-Streptomycin (Gibco), and 10% FBS (Gibco). MLL-AF9 cultures were supplemented with 20 ng ml⁻¹ SCF, 10 ng ml⁻¹ IL-3 and 10 ng ml⁻¹ IL-6 (all cytokines from Peprotech). HoxA9-Meis1 cell cultures were instead grown in 100 ng ml⁻¹ SCF and 5 ng ml⁻¹ IL-3. The cells were co-cultured for 3 days. For flow cytometry of DPP4, the co-cultures were trypsinized and subsequently blocked with murine Fc Block (dilution 1:50) for 10 min at 4° C. Surface staining with anti-CD45-APC/Cy7 (dilution 1:100) and DPP4-PE (dilution 1:20) was then carried out for 30 min at 4° C. The cells were thereafter washed and resuspended in Medium-199 supplemented with 2% FBS with 0.25 μg 7AAD (BD Biosciences) for analysis. Flow cytometry was performed on an LSR II instrument (BD Biosciences) and all data were analyzed using FlowJo (Treestar). For the analysis of DPP4 mean fluorescence intensity, each sample was normalized to a corresponding isotype control antibody-stained sample.

Myeloid Lineage Markers

Bone marrow collected from C57B1/6J mice was stained with a hematopoietic stem and progenitor cell cocktail for 45 min at 4° C. The cells were then washed and resuspended in Medium-199 supplemented with 2% FBS with 0.25 μg 7AAD (BD Biosciences). Flow cytometry was performed on a BD FACS Aria III sorter (BD Biosciences) and all data were analyzed using FlowJo (Treestar).

Reverse Transcription with Quantitative PCR

RT-qPCR was performed to determine levels of Dpp4 mRNA. A total of 1×106 leukemia cells or T cells were lysed and the RNA was extracted using the RNeasy Plus mini kit isolation kit (Qiagen). RNA was subsequently reverse transcribed into cDNA with the SuperScript IV First-Strand Synthesis System (ThermoFisher). The qPCR analysis was performed using iTaq Universal SYBR Green Supermix (Biorad) with primers specific for Dpp4 (forward, ACCGTGGAAGGTTCTTCTGG (SEQ ID NO: 1); reverse, CACAAAGAGTAGGACTTGACCC (SEQ ID NO: 2)) and Gapdh (forward, TGTGTCCGTCGTGGATCTGA (SEQ ID NO: 3); reverse, TTGCTGTTGAAGTCGCAGGAG (SEQ ID NO: 4)). Threshold values (CT) were estimated using CFX Maestro (Biorad) and transcript levels were normalized by subtracting the corresponding Gapdh values. The relative amount of RNA is presented as 2^{-ΔΔCt}.

Droplet-Based Single-Cell RNA Sequencing

WCBM and Image-seq samples were counted in a hemocytometer and encapsulated for a maximum output of 8,000 cells into emulsion droplets using the Chromium Controller (10× Genomics). scRNA sequencing libraries were subsequently prepared using Chromium Single Cell 3 v2 Reagent kits (10× Genomics). Reverse transcription and library preparations were done on a Biorad T100 Thermo Cycler (Biorad). cDNA libraries and final libraries were quantified on an Agilent BioAnalyzer (Agilent Technologies) using a High Sensitivity DNA kit (Agilent Technologies). Libraries were diluted to 4 nM and pooled before sequencing on the NextSeq 500 Sequencing system (Illumina). Pools were sequenced with 75 cycle run kits (26 bp Read1, 8 bp Index1 and 55 bp Read2) to a saturation level of ˜70-80%.

SMARTseq-v4 Library Preparation and Sequencing

Libraries were prepared using the MANTIS Liquid Handler (Formulatrix) and the Biomek FXP Single Arm System with Span-8 Pipettor (Beckman Coulter). Full-length cDNA was prepared using the SMARTseq-v4 Ultra Low Input RNA Kit for Sequencing (Takara Bio) and sequencing libraries prepared using the Nextera XT DNA library preparation kit (Illumina).

The SMARTseq-v4 assay utilizes the SMART technology switching mechanism at the 5′ end of the RNA template to generate full-length cDNA from as little as 10 pg total RNA. The cDNA was assessed for concentration using the Quant-iT Picogreen dsDNA assay kit (Invitrogen, P7589) on the SpectraMax i3 Multi-Mode Detection Platform (Molecular Devices) and normalized to 0.2 ng μl⁻¹ prior to library preparation. Full-length cDNA was fragmented using the Nextera technology in which DNA is simultaneously tagged and fragmented. Tagmented samples were enriched and indexed using 18 cycles of amplification with PCR primers, which included dual 8 bp index sequences to allow for multiplexing (Nextera XT Index Kit). Excess PCR reagents were removed through magnetic bead-based cleanup using PCRClean DX beads (Aline Biosciences) on a Biomek FXP Single Arm System with Span-8 Pipettor (Beckman Coulter). The resulting libraries were assessed using a 4200 TapeStation (Agilent Technologies) and quantified using qPCR (Roche Sequencing). Libraries were pooled and sequenced on a NextSeq Mid Output flow cell using paired, 75 bp reads (Illumina).

10× scRNA-Seq Data Processing

For the 10× scRNA-Seq data, fastq files were obtained using bc12fastq (v1.8.4). Reads were aligned to the mm10 mouse reference genome using the Cellranger pipeline (v3.0.2, 10× Genomics) with default parameters. The obtained read count matrices were further filtered based on two quality metrics: the number of total UMI counts per cell (>800); and the mitochondrial transcript ratio per cell (<0.2). Conos (v1.4.1) 21 was used to integrate multiple scRNA-seq datasets. Each individual dataset was first normalized using the basicP2proc function in pagoda2 (v1.0.10) using default parameters (https://github.com/kharchenkolab/pagoda2/releases/tag/v1.0.10). Different samples were then aligned using Conos with default parameter settings (PCA space with 30 components, angular distance, mNN matching, k=15, k.self=5), and UMAP embedding was estimated using default parameter settings. Leiden clustering (conos::findCommunities) was used to determine joint cell clusters across the entire dataset collection. Quality parameters for the 10× scRNA-seq data are listed in FIGS. 12A-12E.

Differential Expression

For differential expression analysis between cell types in the 10× scRNA-seq data, a Wilcoxon rank sum test, implemented by the getDifferentialGenes( ) function from Conos R, was used to identify statistically significant marker genes that were expressed in each cell cluster. The genes were considered differentially expressed if the P value-determined Z score was greater than 3. For differential expression analysis between Image-seq and WCBM (for example, Image-seq monocytes versus WCBM monocytes), the getPerCellTypeDE( ) function in Conos was utilized.

Cell Annotation

Annotation of the cluster communities was done using marker gene expression. Initial annotations were obtained by entering the top 100 differentially expressed genes in each cluster (ordered by logFoldChange) into the CellKb database65 and further refined by consulting the primary literature referenced therein along with other relevant publications. Specificity of selected markers was additionally confirmed by evaluating expression in the Haemopedia66 and CellMarker67 bone marrow datasets. Markers to classify ten major cell types were identified: B cell (Ms4a1, Ly6d, Cd79a), pre/pro-B cell (Vpreb1, Vpreb3, Dntt), basophil (Mcpt8, Prss34, Ms4a2), dendritic cell (Bst2, Irf8, Siglech, Cox6a2), erythroblast (Cart, Hemgn, Ctse, Cpox, Atpif), monocyte (Lyz2, Ctss, s100a4), monocyte progenitor (Ly6c2, S100a10), diverse progenitors (Cd34, Prtn3, Mpo, Elane, Mpl), granulocyte progenitor (S100a8, S100a9, Cebpe, Fcnb) and neutrophil (Mmp8, Ifitm6, S100a11, S100a8, S100a9).

SMARTseq-v4 Sequencing Data Analysis

SMARTseq-v4 sequencing data were aligned with hisat2 (v4.8.2), using the genome reference mm10. FeatureCounts69 (v1.6.4) was used to calculate read counts. The quality of cells was then assessed based on the number of total counts per cell (library size), requiring at least 500,000 reads per cell. A total of 84 AML and 43 stroma cells were retained for downstream analysis. Seurat was used to analyze the SMARTseq-v4 data, and the quality parameters are listed in FIGS. 12D-12E. In brief, data normalization was first performed using the NormalizeData function with default settings. The top 30 principal components were used to construct shared nearest neighbors graphs and UMAP embeddings. The FindAllMarkers function from Seurat was used to identify differentially expressed (marker) genes for clusters or subtypes. Mesenchymal stromal and endothelial cells were annotated using well-established marker genes: for mesenchymal stromal cells the marker genes were Cxcl12, Pcolce, Ogn and Adipoq; and for the endothelial cells the markers were Eng, Kdr, Plvap and Egfl7 (see also the heatmap in FIG. 13D).

Cell Cycle Signature Score

To assess cell states in different cell subsets and conditions a gene set signature score was used to measure the relative difference of cell cycle states. The signature scores were calculated as average expression values of the genes in a given set. The signature gene list was downloaded from Whitfield et al.70. Hierarchical clustering of cell cycle signature score was used to group AML cells. A two-sided Student's t-test was used to assess differential expression of selected cell cycle genes between proliferating (P) and non-proliferating (NP) cells.

Regressing Out Cell Cycle Genes

Seurat71 (v4.0.6) was used to regress out cell cycle genes. First, each cell was assigned a score, based on its expression of G2/M and S phase markers with the CellCycleScoring function. Then the ScaleData function was applied to regress out the cell cycle genes. The scaled residuals of this model represent a ‘corrected’ expression matrix that can be used downstream for dimensionality reduction. UMAP embedding and graph-based shared nearest neighbor clustering were used with five principal components, and Seurat::FindClusters( ) was used to identify AML cell sub-clusters. Seurat::FindAllMarkers, which utilizes a two-sided Wilcoxon rank sum test to assess statistical significance, was used to find the differentially expressed genes within each sub-cluster.

Analysis of Differentially Expressed Genes

DESeq2 was used to analyze differentially expressed genes between P and NP cells (FIG. 12A), as well as between P+IM (that is, intermediate) and NP cells.

GO Term Enrichment

To test for enriched GO biological processes in gene sets, the ClusterProfiler72 (v4.0.0) package was used to evaluate the enrichment of GO categories in the sets of upregulated and downregulated genes and rank them by adjusted P value (FIG. 17H and FIGS. 12D-12E and 18G). The set of all expressed genes was used as a background.

Analysis of Human AML Data

A regular gene expression correlation analysis was applied to two published, bulk RNA-seq datasets (531 patients for the Oregon Health and Science University, OHSU dataset48, and 188 patients for The Cancer Genome Atlas, Firehose dataset49). Spearman correlation coefficients for each gene with DPP4 were calculated. The top 300 positively correlated genes (based on strength of the correlation coefficient) were determined for each of the two datasets (Firehose and OHSU). Interestingly, a high degree of overlap was observed between the top 300 positively correlated genes from the two datasets. The overlapping gene set was then used for GO analysis.

Multiphoton Imaging Experiments

Image Acquisition

To image the AML cell distribution in different cavities, as well as CellTracker CMTPX retention, two-photon excitation at 900 nm was used, and the emission was collected at 340-460 nm to detect the second-harmonic generation signal of collagen (bone), while 500-550 nm was used to detect the AML-GFP signal. The bone front staining and CMTPX were excited at 775 nm and the resulting fluorescence collected using 525/50 nm (tetracycline) and 617/73 nm (Alizarin Red, CMTPX) filters. All image stacks were acquired using a previously described microscope51,61, with a 2 μm step size from the calvaria surface, and 20 frames from the live scanning microscope (30 frames per second) were averaged to acquire a single image.

For imaging the stroma, β-actin-GFP mice, DPP4 expression in vivo, as well as for quantifying AML proliferation, the home-built microscope described above was used, with an imaging wavelength of 980 nm. Emission signals were collected with filter 439/154 for the second-harmonic generation signal, filter 525/50 for GFP and filter 607/70 for the AF568 signal with a combination of FF705 LP, FF495 LP and FF552LP dichroic mirrors. For imaging the CXCL12-DsRed stroma, two-photon excitation at 980 nm was used, and emission signals were collected using the filter 439/154 for the second-harmonic generation signal (blue channel, shown as gray in the Figures) and 585/40 for DsRed (red channel) with the same dichroic mirrors as listed above. Image stacks were acquired with a step size of 2 μm, as well as a 15-frame average.

For displaying the data, some images were background-subtracted with the mode of the stack histogram (corresponding to the noise pixel intensity) and subsequently filtered using the 3D Fast Filters (median) function in FIJI with an x, y and z radius unit of 1. The brightness and contrast of images in the figures were adjusted, but in all cases the image analysis was performed using the raw data.

Image Stitching and Maximum Intensity Projections

Large area images were obtained by stitching together images from individual microscope fields of view sequentially for each z plane using the Grid/Collection stitching plugin in Fiji and using an overlap of 30%. Maximum intensity projection images were obtained using the Z Project function (Fiji).

Characterization of P, NP and IM Cells

The same bone marrow cavities in the same animals were imaged both on day 1 and day 3 after transplantation of AML cells. Identical cavities on day 3 were found using the recorded coordinates with respect to the bregma and lambda reference points, and comparison of the signal distribution and specific landmarks in the second-harmonic generation channel. The number of AML cells was quantified at both timepoints, and the fold-change in AML cells between day 1 and day 3 was calculated for each cavity:Fold-change=(no. of cellsday 3−no. of cellsday 1)/(no. of cellsday 1).

Cells were then grouped based on their fold-change as either proliferating (P, fold-change >2), intermediate (IM, fold-change>0 and ≤2), or non-proliferating (NP, fold-change≤0), which corresponded to average cell numbers of 29.6 (with a 99.9% confidence interval of 21.7 to 37.4 cells), 13.2 (with a 99.9% confidence interval of 5.5 to 20.8 cells), and 2.1 (with a 99.9% confidence interval of −1.2 to 5.4 cells), respectively, on day 3.

Analysis of Cell Tracker Labeling

AML cells were labeled with CellTracker Red (CMTPX, 10 μM, ThermoFisher Scientific) before transplantation. In brief, the cell suspension (in Ca2+/Mg2+ free PBS containing 10 μM CMTPX) was incubated at 37° C. for 45 min. Cells were then pelleted to remove the staining solution and resuspended in 300 μl PBS for retro-orbital injection. The mean CellTracker intensity was measured on day 3 after transplantation and measured at the brightest plane of the cell. The cells were considered positive for CMTPX when the measured signal was greater than 12.5 (the background noise in the marrow cavity). The number of CMTPX-positive cells was then divided by the total cell number sampled from the bone marrow cavities harboring the same cell counts (n=7 mice).

Analysis of DPP4 Expression In Vivo

DPP4 antibody (BioLegend) and Isotype control antibody (BioLegend) were conjugated to AF568 using the Lightning Link kit (Abcam). Antibody and isotype were injected retro-orbitally 1 h prior to the imaging session at a dosage of 1 mg kg⁻¹. Images were collected with an excitation wavelength of 980 nm (1.6 nJ pulse energy, filter set detailed above), using a z-step of 2 μm with a 15-frame average on the custom-built microscope described above.

AML cells were segmented based on the GFP signal in the obtained images. For this, seeds were generated using the interactive watershed tool (Fiji) and used as input for the 3D-Watershed segmentation plugin (Fiji). The images were thresholded to generate a mask. This was used to calculate the total AF568 signal, as well as the total GFP signal, in each cell using the red and green channels of the images, respectively, as input for the 3D Object counter plugin (Fiji). The ratio of red to green fluorescence (multiplied by 10) for each cell was plotted in FIG. 17C both for the DPP4 and isotype control cells.

Distribution of AML Cells in D-, M- and R-Type Cavities

The protocols to determine the bone remodeling status have been described previously. The first calcium-chelating reagent dye 1 (Tetracycline, Sigma, 35 mg kg⁻¹) was given i.p. 48 h prior to imaging to label the bone fronts and track the bone resorption activities over the course of 2 days. The second calcium-chelating reagent dye 2 (Alizarin Red, 40 mg kg⁻¹) was injected on the day of imaging to label all of the bone fronts. The bone remodeling status was then defined based on the ratio of dye 1 to dye 2 in a single bone marrow cavity (the concave endosteum), and therefore the bone marrow cavities were classified as: deposition type (D-type; dye 1:dye 2 ratio>75%); resorption type (R-type; dye 1:dye 2 ratio<25%), or mixed type (M-type; dye 1:dye 2 ratio 25-75%. The distributions of seeding and expansion of AML cells were then mapped with respect to the D-, M- and R-type cavities on day 0 (3 h after transplantation), day 1 and day 3 after transplantation. The same mouse was followed up longitudinally on day 0 and day 1 and the cavity type was defined on day 0. A separate cohort of animals was used for day 3 to avoid unwanted inflammatory responses from the survival surgical procedures.

Animal Handling

Male and female 8-week-old C57B16/J mice (cat. no. 000664) or male and female CXCL12-DsRed mice (cat. no. 022458) were ordered from the Jackson Laboratory, housed in an animal facility for at least 2 weeks and used for experiments at between 10 and 14 weeks of age. The 8-week-old female Fezh/J mice B6J.129(Cg)-Gt(ROSA)26Sortm1.1(CAG-cas9*,−EGFP)Fezh/J were ordered from the Jackson Laboratory (cat. no. 026179) and used in experiments. Male and female β-actin-GFP mice (Jackson Laboratory, cat. no. 006567) were bred in-house and used between 10 and 16 weeks of age. Male and female β-actin-DsRed mice (Jackson Laboratory, cat. no. 006051) were bred in-house and used between 10 and 16 weeks of age. The β-actin luciferase (βact) mice from Taconic (cat. no. 11977) were bred with ubiquitin-c-GFP (UcGFP) mice from the Jackson Laboratory (cat. no. 004353) to generate βact-UcGFP transgenic mice. An 8-week-old female βact-UcGFP mouse was then used to generate the HA9M1 cell line. All mice were housed in the pathogen-free Massachusetts General Hospital (MGH) Animal Facilities, which were equipped with ventilated micro-isolator cages. Sentinel programs and veterinary oversight were in place. Mice were given standard chow and drinking water ad libitum. An automated 12 h dark-12 h light cycle was observed and mice were housed at a fixed temperature (21° C.) and humidity (66%). The MGH Animal Facility is under the supervision of the MGH Center for Comparative Medicine. All facilities are fully accredited by the Association for Assessment and Accreditation of Laboratory Animal Care International (000809) and meet the National Institutes of Health standards as set forth in the Guide for Care and Use of Laboratory Animals by the Department of Health and Human Services. All procedures involving animals were carried out in agreement with protocols 2012N000190, 2007N000148 or 2016N000085 approved by the Institutional Animal Care and Use Committee of Massachusetts General Hospital.

Statistics and Reproducibility

P<0.05 was considered significant unless specified otherwise.

### Example 2

System Requirements

Imaging Modality

Either a confocal or multiphoton microscope can be used to visualize the 3D spatial organization of bone marrow (BM) tissue.

Ablation Modality

Requires a scanning device that steers the laser ablation beam across the microscope field of view. This has similar characteristics as the scanning optics used in some multiphoton microscopes (in fact, in this system, the same scanning optics are used for imaging and laser ablation). The ablation laser should fulfill the following requirements: repetition frequency between 100 kHz to 5 MHz (the latter is used on our system) and a pulse energy of ˜50-100 nJ. Note that while we typically use between 10-20 nJ pulse energy for the Image-seq procedure, the laser itself should have higher pulse energies because typical optical systems incur energy losses as the beam propagates to the image plane of the microscope. If the ablation geometry can't be controlled by arbitrarily positioning the scanning angle of the device, a variable aperture should be inserted into the intermediate image plane to control the ablation geometry.

Flushing System

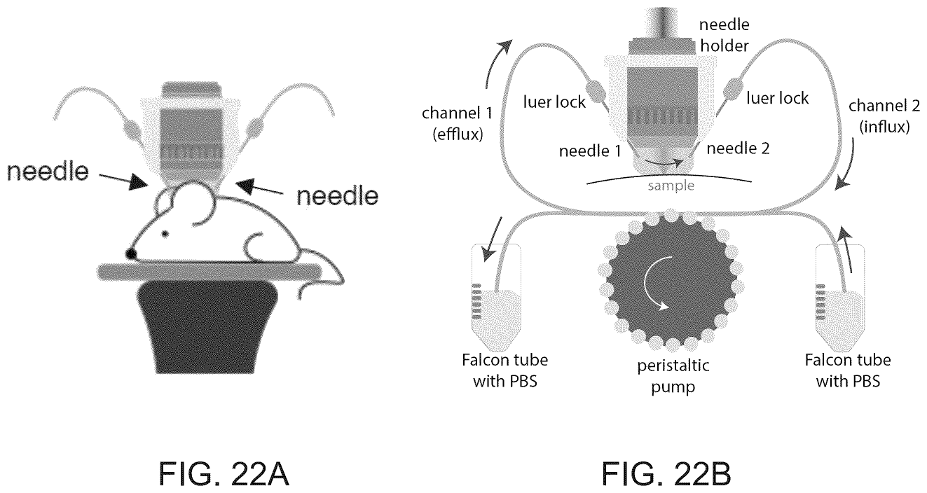

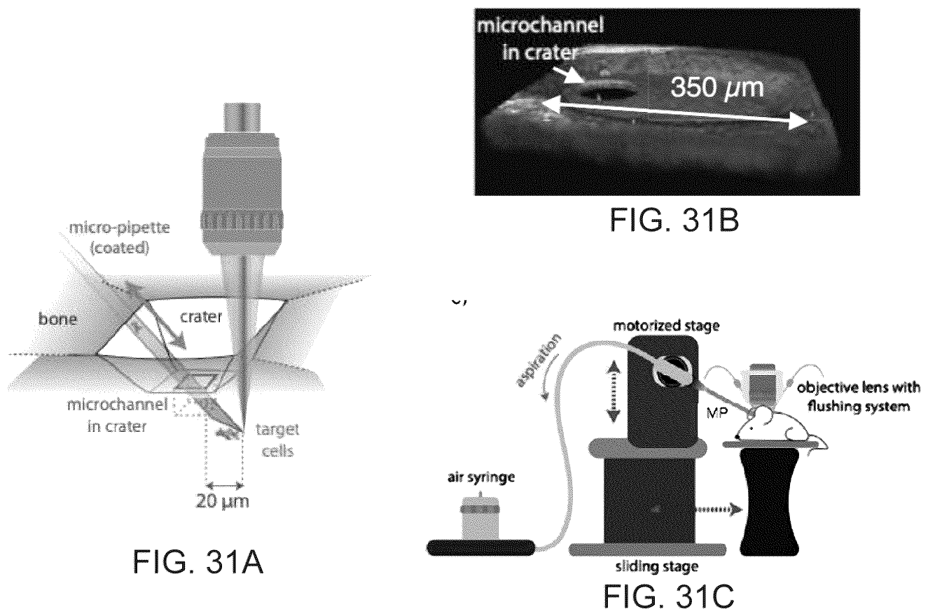

A flushing system should be installed on the objective lens of the multiphoton/confocal microscope, as shown in FIG. 22B. The holder secures two blunt-tip needles (22G, Grainger scientific, 5FVC4) across from one another (efflux from one needle is the influx to the other needle, see FIGS. 22 and 31C) so that a steady stream of PBS or saline can flow across the sample during the ablation procedure and remove any gas and debris that is generated. Flow is achieved using a dual channel peristaltic pump (Cole-Parmer, EW-78001-58) with tubing (ISMATEC, SC0330), needles are attached to one end of the tubing.

Micropipette Assembly for Micropipette

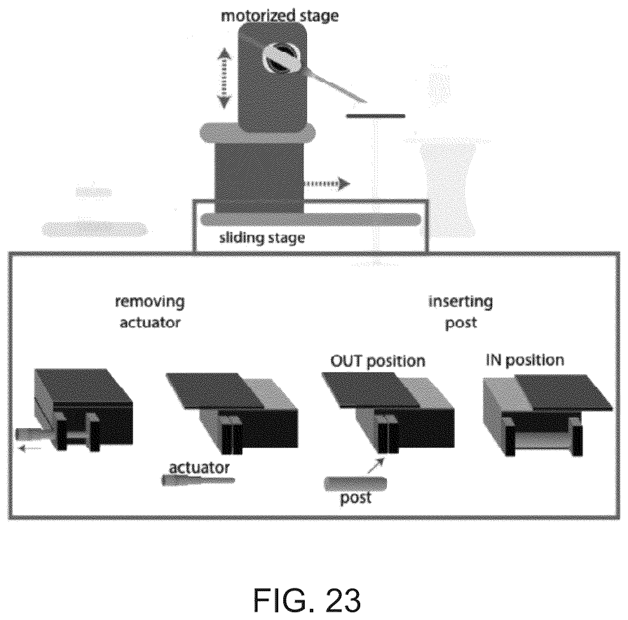

The micropipette holder should be mounted onto a motorized micromanipulator (Sutter Instrument, MPC385), and connected to an air syringe (Cooper Surgical, 6-34-520) with polythene tubing (Cooper Surgical, 6-34-536). To aid with quickly translating the micropipette to the bone marrow and pulling it back out to eject the collected sample into a tube, the micromanipulator should be mounted onto a sliding stage. A sliding stage can be made by removing the actuator from a translational stage and sliding in a post (L=50 mm) as a spacer, thereby achieving an IN (post in) or OUT (post out) position (see FIG. 23). We recommend using a 50 mm travel linear translation stage (Thorlabs, XR50P). It should be positioned so that the micropipette appears in the center of the field-of-view when the micromanipulator is moved to the “IN” position.

Visualizing the Tissue for Image-Seq

Multiple contrast mechanisms can be used to visualize the procedure and reconstruct the 3D spatial position of the extracted cell sample, including autofluorescence, confocal reflectance or labeling with fluorescent membrane dyes such as DiD/DiR/DiI/Di8-ANEPPS or a fluorescent antibody. If a single cell or a specific cell population is to be isolated, then either transgenic expression or fluorescent antibody labeling of this subpopulation is necessary so that after aspiration of the target cell(s) and 100-400 surrounding cells, the target cells can be isolated by flow cytometric sorting. For transplantation models, cells can be labeled prior to injection. If not working with a fluorescent reporter mouse or using confocal reflectance to visualize the tissue, we recommend injecting Di8-ANEPPS (1.9 mg/kg animal weight) prior to the Image-seq procedure.

IACUC Approval

Prior to performing any of the experiments detailed below, an animal protocol describing these procedures should be submitted to and approved by the institutional animal care and use committee (IACUC). Experiments should be carried out in accordance with the guidelines set forth for the procedures on rodents at the home institution. Administer analgesics in accordance with an institution's IACUC policy and consult an OAR/IACUC veterinarian.

In Situ Imaging of Long Bones

Materials:
Standard surgical kit (for example Fisher Scientific, 50-822-920)
Kimtech wipes (Fisher Scientific, 06-666)
Microscope slide (Fisher Scientific, 12-550-15)
Modelling clay (Amazon, B0025Z8H7Q)
Tibia (or femur) bone
Razor blade (ThermoFisher, S17302)

A. Euthanize the animal and dissect the tibia (or femur bone, or any other type of bone) from the animal. Use Kimtech wipes to remove excess muscle tissue from the isolated bone.

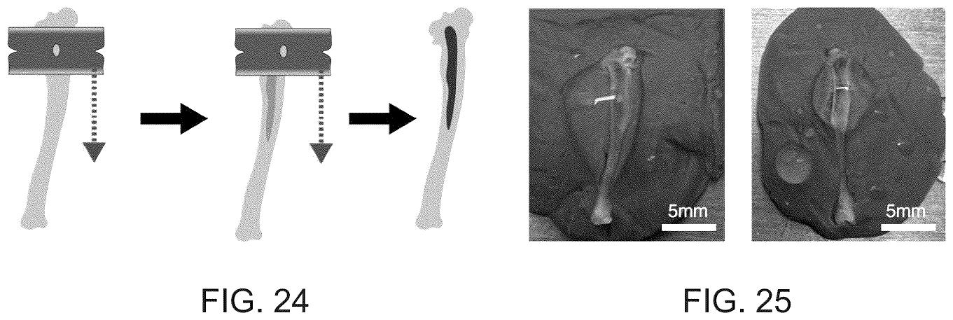

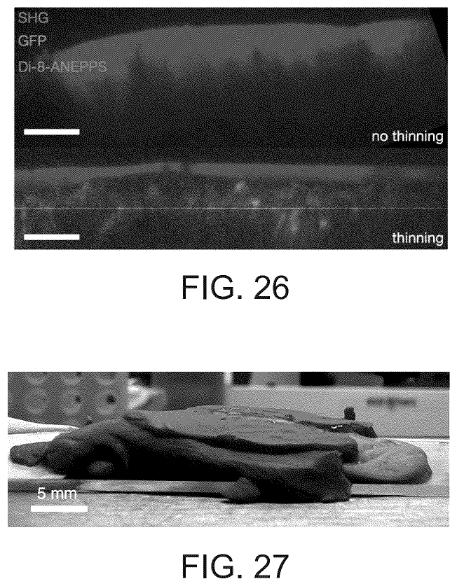

B. Carefully scrape off superficial bone tissue from the bone surface with a razor blade (or a micro drill) until the bone marrow becomes visible (see FIGS. 24 and 25), this typically occurs at a bone thickness of ˜50 μm. Hydrate the bone using a drop of PBS to maintain cell viability. Note:—Make sure the bone marrow is not damaged during this step.—Aim for a bone tissue thickness of ≈50 μm or less (see FIG. 26)

C. Mount bone sample on a microscope slide by gently pressing the bone into the modelling clay (blue in FIG. 4 above). Deform the modelling clay so that the area to be imaged is parallel to the image plane of the microscope and the glass slide (see FIG. 27). Note: Alternatively, instead of modelling clay, paraffin can be used as a mounting material. However, once the paraffin has hardened it is more challenging to deform.

In Vivo Imaging of the Calvarium

Materials:
Standard surgical kit (for example Fisher Scientific, 50-822-920)
Anesthetics and analgesics in accordance with an institution's IACUC
Mouse holder with integrated heating pad (drawing available upon request)
Razor blade (ThermoFisher, S17302)
Ethilon 6-0 nylon monofilament sterile sutures (Zogo Medical, ETH-1956G)
Curad antibiotic ointment (ATC medical, PF11102)
Betadine swabsticks (Fisher Scientific, 19-061617)
Sterile cotton swabs (Fisher Scientific, 18-366-472)
Phosphate-buffered saline, PBS (ThermoFisher Scientific, 10010002)

Procedure

1. Prior to beginning the experiment, install the correct dichroic mirrors and filters for visualizing and separating the emission spectra of the fluorophores of interest. Tools like ThermoFisher's Fluorescence spectra viewer or BioLegend's spectra analyzer can be used to find optimal choices.

2. Administer analgesics ˜30-60 min prior to imaging (we use buprenorphine IP at 0.05-0.1 mg/kg animal weight). Anesthetize the mouse (we use vaporized isoflurane at 3-4% for induction and 1-2% for maintenance), ensuring depth of anesthesia by toe pinch. Hydrate the mouse's eyes.

3. Transfer the mouse to a holder with integrated heating pad and anesthesia supply.

4. Trim the hair around the incision site using a sterile razor blade or surgical scissors and scrub with betadine.

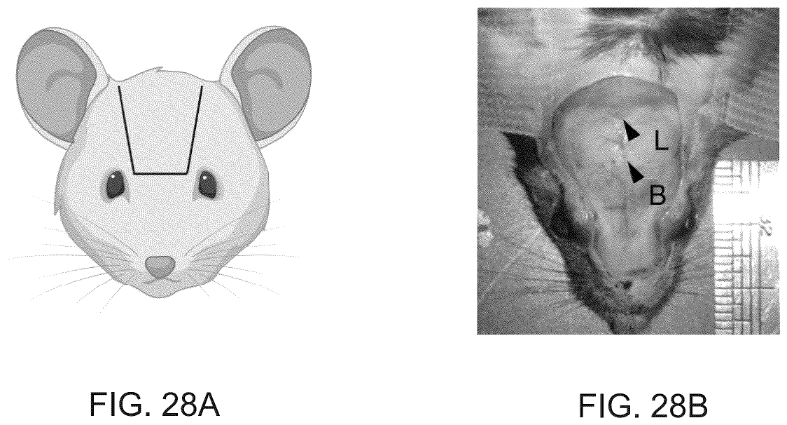

5. Make a ˜5 mm×7 mm incision into the skin (see FIG. 28A) using sterile surgical scissors, taking care to point their tip upwards to avoid damaging the skull or eyes.

6. Fold back the skin flap, secure it to the back of the skull using a drop of antibiotic ointment and expose the skull bone. Hydrate it with a drop of sterile PBS, taking care to keep the skin flap hydrated as well.

7. Remove the periosteum by gently rubbing two sterile cotton swabs across the skull in a concerted motion, starting from the interfrontal/sagittal suture and moving the swabs outwards.

8. Transfer the mouse to the multiphoton/confocal microscope. Secure in such a manner that the skull surface is mounted parallel to the image plane of the microscope.

9. Record the position of lambda and bregma (see FIG. 28B): they will serve as reference points for longitudinal imaging experiments.

10. Perform IVM experiments and record the position of the imaging site with respect to Bregma and Lambda. We often record z-stacks with a 2 μm step size and 15-30 frame average. To study short-term dynamics, it is helpful to perform time-lapse microscopy and record images or short z-stacks with a time interval of a few seconds or minutes, or up to a few hours. If this is a terminal experiment, sacrifice the animal after finishing the imaging session.

For Survival Experiments:

11. Once the imaging session is completed, flush the skull extensively with sterile saline. Close the scalp with surgical sutures and infiltrate 0.25% Bupivacaine (2 mg/kg) into the surgical site to aid with pain management. Finally, apply triple antibiotic ointment (Curad) on the sutured area. Return the animal to its cage and monitor it until awake. Administer buprenorphine (0.05-0.1 mg/kg animal weight) either IP or SQ along with topical antibiotic ointment every 8-12 hours for up to two days after the surgery.

Image-Seq Procedure

Equipment:
Peristaltic pump (Cole-Parmer, EW-78001-58)
Confocal/Multiphoton microscope
Ablation modality (see section 1)
Air syringe (Coopersurgical, 6-34-520)
Polythene tubing (Cooper Surgical, 6-34-536)
Micromanipulator (Sutter Instrument, MPC-385)
Sliding stage (see section 1)

Materials:
Sigmacote (Sigma-Aldrich, SL2-100ML)
Non-stick, RNAse free microfuge tubes (ThermoFisher Scientific, AM12450)
Micropipette: 20°-angled blastomere biopsy pipette, 32-37 μm ID (Origio/Cooper Surgical, MBB-FP-M20)
DNAse/RNAse-free deionized water (ThermoFisher Scientific, 10977015)
70 kDa MW fluorescent dextran, for example Tetramethylrhodamine (ThermoFisher Scientific, D1819)
Falcon tubes (Fisher Scientific, 14-432-22 and 05-527-90)
Medium 199 (Gibco, 11150059)
Fetal bovine serum (FBS, Gibco, A3160501)
RNAse away (Fisher Scientific, 21-236-21)
Di8-ANEPPS (ThermoFisher Scientific, D3167)
Phosphate-buffered saline, PBS (ThermoFisher Scientific, 10010002)
Sterilized (autoclaved) surgical equipment and perfusion tubing (ISMATEC, SC0330)
Collagenase I (Sigma, C0130)
Collagenase XI (Sigma, C7657)
Hyaluronidase (Sigma, H3506)
DNase I (Sigma, D5319)
EDTA (ThermoFisher Scientific, AM9260G)
25G Hypodermic Needles, (Fisher Scientific, 14-826AA)

Permanent Marker

Prior to Executing the Image-Seq Cell Isolation

A. Coat the micropipette to prevent cells from sticking to the glass surface of the micropipette it is necessary to coat it with a siliconizing agent that prevents protein adhesion. It is also recommended to fluorescently coat the pipette for visualization by multiphoton/confocal imaging. This can be achieved as follows:

Install a fresh set of tubing onto both channels of the peristaltic pump described above, attaching the micropipette to one end. Hold the micropipette into a Falcon tube filled with 1-5 mL of Sigmacote. Allow the Sigmacote to flow into the micropipette through its tip, setting the flow rate to ˜200 μl/min, and allowing it to flow continuously for 2 min. The waste can be collected by inserting the other end of the tubing into an empty Falcon tube.

Transfer the micropipette to the other channel and flush it with sterile, deionized (RNAse-free) water using the same flow speed and direction. We typically coat many micropipettes at once and store them for future use.

Prior to experiment, install the micropipette on multiphoton microscope, and flow a fluorescent dextran with a color of choice into the micropipette using the air syringe. Pipette the dextran up and down for 2-3 min before expelling the liquid and gently drying the tip with a piece of lens tissue. The micropipette is now ready for use. Note that we do this final step directly before the experiment.

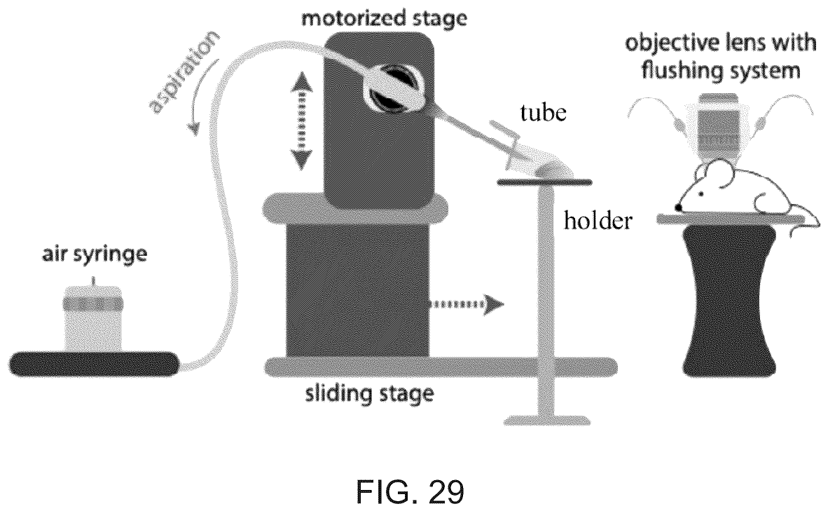

B. Prepare microfuge tubes for ejecting the samples: Clean the workspace with RNAse Away. Pipette 5 μl of Medium-199 supplemented with 2% FBS into enough microfuge tubes to collect all micropipette samples. Store them on ice. Clean the microfuge holder (FIG. 29) with RNAse Away.

C. Visualization: If not working with a fluorescent reporter mouse/fluorescent transplant model, it is necessary to inject either Di8-ANEPPS (1.9 mg/kg animal weight) or a fluorescent antibody to aid in the visualization of the Image-seq procedure and/or label the target cell population. Wait at least 30 min after injection to ensure proper labeling.

D. Perfusion: Unless doing in vivo cell isolation, mice should be perfused prior to sample isolation. This minimizes the RBC content and further processing steps. General procedures for rodent perfusion have been described in detail elsewhere, along with relevant videos. We detail our adaption below:

Prepare 40 ml of 5 mM EDTA/PBS in a Falcon tube, along with 40 ml of regular PBS (w/o Ca/Mg).

Store both solutions on ice.

Set the flow rate of the peristaltic pump to 5 ml/min and attach a 25G needle to one end of the tubing

4. Use a permanent marker to mark the needle ˜5 mm from its tip (note that the exact distance depends on the size, and therefore the age and gender, of the mouse heart). This is to ensure that the needle is not injected too deeply into the mouse heart during the perfusion procedure.

Transfer mouse to a dissection tray and set the anesthesia to 4-5% vaporized isoflurane.

Shave the chest.

Place one end of the peristaltic tubing into the solution of EDTA/PBS, turn on the pump and wait until liquid is flowing out of the syringe. Turn off the pump.

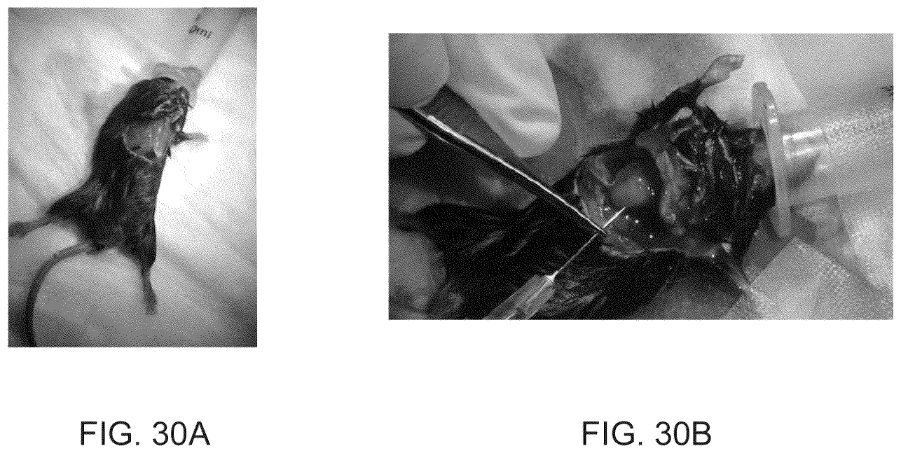

Scrub the chest with betadine, make an incision and cut off the skin in the region surrounding the ribcage (FIG. 30A). This is to aid in the visualization of the procedure.

Cut open the ribcage parallel to the sternum, at a distance of ˜10 mm on either side. Fold up the ribcage towards the mouse head and gently move the beating heart with a pair of tweezers to expose the right atrium. Make an incision into the right atrium and ensure that blood is flowing into the chest cavity.

Use a pair of tweezers to gently reposition the heart and facilitate insertion of the needle into the apex of the left ventricle. Turn on the pump and hold the needle in place with a pair of tweezers (FIG. 30B).

Perfuse with 10 ml of ice-cold PBS/EDTA followed by 10 ml of ice-cold PBS.

Mount the mouse in a holder or dissect the calvarium/tibia and mount it onto a glass slide as described in the section for in situ imaging of long bones described above.

E. Perfusion for the isolation of stromal cells: This procedure is nearly identical to the one described above. However, instead of perfusing with ice cold EDTA/PBS, the mouse is first perfused with 10 ml EDTA/PBS at 37° C., then with 10 ml of an enzymatic digestion buffer at 37° C. This buffer consists of 450 U/mL Collagenase I (Sigma), 125 U/ml Collagenase XI (Sigma), 60 U/ml Hyaluronidase (Sigma), 60 U/ml DNase I (Sigma) in 20 ml of Medium199 (Gibco). The mouse is then incubated at 37° C. for 20 min and the calvarium or long bone dissected and mounted.

Laser Ablation and Cell Aspiration by Micropipette

After positioning the sample (this can be either a live mouse or a mounted calvarium/long bone) in the image plane of the microscope and finding the target location, turn on the peristaltic pump and flow PBS across the sample at a flow rate of 10 ml/min.

A. Check the thickness of the bone by translating the sample along the z-dimension, using the second harmonic generation signal (SHG signal, collected at half the wavelength of the multiphoton laser) to visualize the bone.

B. Set the laser pulse energy to 14 nJ at the sample and thin a 200×300 μm area of bone, leaving a layer that has a minimum thickness of 20 μm, thereby ensuring minimal damage to the bone marrow located beneath (see FIG. 31B for a 3D representation of what the generated ablation crater typically looks like). The ablation procedure itself is performed at a rate of 0.25 μm/670 ms along the z-dimension, which corresponds to 10 passes per plane using the 15 frame per second imaging rate of our optical system.

C. Set the laser pulse energy to 11 nJ and ablate a ˜60×100 μm microchannel (FIG. 31A-31B) that creates access to the bone marrow tissue, taking care to remove all bone but to avoid ablating bone marrow tissue. In doing so, make sure to position the top of the ablation crater at a distance of 20-30 μm from the target cell(s) (see FIG. 31A).

D. Move the sample down (z-dimension) and slide in the micropipette (“IN” position), visualizing its tip in the microscope field-of-view.

E. Move the sample (=mouse in FIG. 31C) back up and position the micropipette exactly above the microchannel but without touching it. Using the micromanipulator, insert the micropipette through the microchannel and position it next to the target cell(s) (see FIG. 31A). Note that the micropipette has a ˜20° angle with respect to the sample plane (see FIG. 31A and FIG. 31C).

F. Aspirate the target cells while recording a video by gently suctioning with the air syringe (FIG. 31C). The amount by which the air syringe is rotated controls the overall volume that is displaced and therefore the total number of cells that are aspirated.

G. Move the sample down and slide out the micropipette (“OUT” position).

H. Place a microfuge tube filled with Medium-199/FBS onto a holder and insert the micropipette into the tube liquid. Expel the aspirated cell sample, directly generating a single-cell suspension (see FIG. 29).

I. Remove the tube and place it on ice.

J. Repeat the procedure to collect more Image-seq samples. To ensure high-quality single-cell data, ensure that the entire procedure (from mouse perfusion to multiple sample pickings) does not take longer than ˜2.5 h. In this time-frame we typically collect 4-6 samples.

In general, the number of Image-seq samples that are required to obtain statistical significance is given by the size of the biological effect and the observed standard deviation. If there is no sequencing data available in the literature to estimate the size of the biological effect, we recommend running a small pilot with N=3 Image-seq samples to quantify it along with the standard deviation, and, if necessary, to collect additional samples until statistical significance is achieved. Ideally samples should be collected from at least 3 different mice. Depending on the biological question, use either the 10× genomics platform for cell encapsulation and library preparation or the SMARTseq-v4 protocol

10× Procedure

Materials:
Image-seq sample(s) isolated in section 6
Medium 199 (Gibco, 11150059)
Ambion UltraPure Bovine serum albumin (Fisher Scientific, AM2616)
Gibco Trypan Blue solution, 0.4% (Fisher Scientific, 15-250-061)
Hemocytometer (iNCYTO, DHC-B02)
Low binding pipette tips, p20 (Rainin, 30389226)
10× Chromium Single Cell analysis kit of choice

### Procedure

A. All of the droplet-based sequencing data generated for the Image-seq paper used 10× Genomics v2 kits that are no longer available for purchase. Depending on the sequencing needs consult with local sequencing core or 10× Genomics representative regarding what type of 10× Chromium Single Cell analysis kit needed.

B. Check the final volume in each microfuge tube from the Image-seq cell isolation procedure described above. At this stage there are two considerations. Firstly, the sample should be in a volume that is compatible with the 10× Chromium Single-Cell analysis kit of choice. Secondly, the overall cell number needs to be determined. For counting cells in the disposable hemocytometer, the minimal required volume is 5 μl. Adjust the volume of the samples so that 5 μl can be safely removed without depleting the sample for the downstream sequencing step. If sample size is not a limiting factor, a larger volume of the sample can be used to perform the cell count.

C. Mix the cell suspension in a 1:1 ratio with 0.4% trypan blue.

D. Load the cells onto the hemocytometer (iNCYTO) and count live and dead cells. Also, take note if there are cell aggregates. If there is extensive aggregation, additional dissociation of the sample could be required to avoid clogging the 10× Chromium Chip.

E. Consult the cell loading table in the Chromium Single Cell 3′ user guide. Using 0.4 mg/ml ultra-pure BSA in Medium 199 adjust the cell concentration accordingly.

F. Place the cells on ice until ready to load on the 10× Chromium Chip.

G. For preparation of the scRNA-seq samples, follow the instructions provided by 10× Genomics kit.

H. In order to prepare scRNA-seq libraries for sequencing, consult with sequencing core or provider to determine preferred submission format.

SMARTseq-v4 Procedure

If SMARTseq-v4 library preparation is chosen for processing the samples, it is necessary to first sort single cells into individual wells of a 96-well plate using a flow cytometer or microfluidic sorting device. To aid with drawing the correct gates, it is recommended to collect whole bone marrow preparations from one of the bones that were not processed by Image-seq. For our experiments, the Bauer Core at Harvard University performed library preparation and sequencing using the SMARTseq-v4 Ultra Low Input RNA kit. It is recommended that using a core facility or a company to perform library preparation and sequencing since the procedure can be time-consuming and tedious.

Equipment:

Flow Cytometer

Any equipment detailed in the SMART-Seq® v4 Ultra® Low Input RNA Kit for Sequencing User Manual.

Materials:
Image-seq sample(s) isolated in the section describing the Image-Seq procedure above
Optional: antibody for staining a subpopulation of cells
Medium-199 (Gibco, 11150059)
FBS (Gibco, A3160501)
DAPI (BioLegend, 422801)
Phosphate buffered saline, PBS (ThermoFisher Scientific, 10010002)
SMARTseq-v4 Ultra Low Input RNA Kit for Sequencing (Takara Bio, 634891)

Any further reagents listed in the SMART-Seq® v4 Ultra® Low Input RNA Kit for Sequencing User Manual.

Sort

A. Pipette sample up and down several times to generate a single-cell suspension.

B. Stain sample with antibody for 30 minutes at 4° C. in Medium 199 supplemented with 2% FBS, using the antibody concentration recommended by its manufacturer.

C. Add 1 ml of PBS to the sample, as well as 0.1 μg of DAPI and incubate the sample for 10 min.

D. Gently vortex the cell sample and transfer it to the flow cytometer (MoFlo Astrios EQ cell sorter for our experiments).

E. Use a whole bone marrow preparation processed in the same manner to draw initial gates for flow cytometric sorting.

F. Sort single, live cells into individual wells of a 96-well PCR plate filled with 2.6 μl of lysis buffer (Takara Bio USA, Inc.).

G. After completing the sort, seal plates, spin them down and snap freeze them

H. Store plates at −80° C. prior to preparation for cDNA synthesis using the SMARTseq-v4 assay. Note that it is possible to bank samples until a sufficient number of cells have been collected and perform library preparation on all of the collected cells simultaneously.

Library Preparation

A detailed description of the cell-lysis, reverse transcription, library preparation and indexing steps can be found in the SMART-Seq® v4 Ultra® Low Input RNA Kit for Sequencing User Manual by Takara Bio.

Sequencing

In order to prepare scRNAseq libraries for sequencing, consult with sequencing core or provider to determine preferred submission format.

Data Analysis

The 10× scRNA-seq data and SMARTseq-v4 data described in the Image-seq manuscript have been deposited into the Gene Expression Omnibus (GEO) database under GSE188902.

Operating System

This protocol assumes users have a Unix-like operating system (i.e., Linux or MacOS X), with a bash shell or similar. All commands given here are meant to be run in a terminal window. The R introductory message will start with a ‘>’ prompt.

Align the scRNA-Seq Reads to the Genome

For the 10× scRNA-Seq data, Reads were aligned to the mm10 reference genome using the Cellranger pipeline (version 3.0.2, 10× Genomics). For SMARTseq-v4 data, reads were aligned with hisat219 and featureCounts20 was used to calculate read counts.

A. Map the 10× scRNA-seq reads for each sample to the reference genome and read counts quantification:

FQ = =${INPUTDIR}
cellranger count --id $FQ \
--fastqs=$FQ / \
--transcriptome=refdata-cellranger-mm10-1.2.0 \
--localcores=50 \
--localmem=250

B. Map the SMARTseq-v4 sequencing data for each cell to the reference genome and read counts quantification:

OUTPUT=${SAMPLE}.genome
FQ1=${INPUTDIR}/${SAMPLE}_R1_001.fastq.gz
FQ2=${INPUTDIR}/${SAMPLE}_R2_001.fastq.gz
hisat2 -t \
-x ${REF} \
-1 ${FQ1} \
-2 ${FQ2} \
--rg-id=${SAMPLE} --rg SM:${SAMPLE} --rg LB:${SAMPLE} \
--rg PL:ILLUMINA --rg PU:${SAMPLE} \
--new-summary --summary-file ${OUTPUT}.log \
--met-file ${OUTPUT}.hisat2.met.txt --met 5 \
-k 10 \
--secondary \
--seed 12345 \
-p 4 -S ${OUTPUT}.bam
featureCounts -T 5 -p -t exon -g gene_name -a $gtf -o ${SAMPLE}
${INPUT_DIR}/${SAMPLE}.genome.bam
10X scRNA-seq data integration

We used Conos21 (1.4.6) to integrate multiple scRNA-seq datasets together. Each individual dataset was first normalized using basicP2proc function in pagoda2²² with default parameters. Different samples were then aligned using Conos with default parameter settings.

```
  > library(pagoda2)
  > library(conos)
  > raw <- readRDS(‘raw.rds’) # read list object of count matrices
  > p2lis2 <- lapply(raw,function(x) basicP2proc(x,n.cores = 10,min.transcripts.per.cell
=0))
  > con <- Conos$new(datlp2, n.cores = 10)
  > con$buildGraph( )
  > con$findCommunities( )
  > con$embedGraph(method = ″UMAP″, spread = 7)
  > def <- con$getDatasetPerCell( ) # get differentially expressed genes
  SMARTseq-v4 data integration
  Seurat (Version 4.0.6) was used to analyze the SMARTseq-v4 data.
  > library(Seurat)
  > marrow <- CreateSeuratObject(counts = cds)
  > marrow <- NormalizeData(marrow)
  > marrow <- FindVariableFeatures(marrow, selection.method = ″vst″)
  > marrow <- ScaleData(marrow, features = rownames(marrow))
  > marrow <- RunPCA(marrow, features = VariableFeatures(marrow), ndims.print = 6:10,
nfeatures.print = 10)
  > marrow <- FindNeighbors(marrow, dims = 1:30)
  > marrow <- FindClusters(marrow, resolution = 1)
  > marrow <- RunUMAP(marrow, dims = 1:30)
  > DimPlot(marrow, reduction = ″umap″)
```

Regressing out cell cycle genes Seurat (version 3) was used to regress out cell cycle genes. First, we assigned each cell a score, based on its expression of G2/M and S phase markers with the CellCycleScoring function. Then we applied the ScaleData function to regress out cell cycle genes.

```
  > library(Seurat)
  > exp.count <- readRDS(‘exp.count.rds’)
  > marrow <- CreateSeuratObject(counts = exp.count)
  > s.genes <- cc.genes$s.genes
  > g2m.genes <- cc.genes$g2m.genes
  > marrow <- NormalizeData(marrow))
  > marrow <- FindVariableFeatures(marrow, selection.method = ″vst″)
  > marrow <- ScaleData(marrow, features = rownames(marrow)
  > marrow <- CellCycleScoring(marrow, s.features = s.genes, g2m.features = g2m.genes,
set.ident = TRUE)
  > marrow <- ScaleData(marrow, vars.to.regress = c(″S.Score″, ″G2M.Score″), features =
rownames(marrow))
  > marrow <- RunPCA(marrow, features = VariableFeatures(marrow), nfeatures.print =
10)
  > marrow <- FindNeighbors(marrow, dims = 1:10)
  > marrow <- FindClusters(marrow, resolution = 1)
  > marrow <- RunUMAP(marrow, dims = 1:5)
  Differential expression
```

DESeq2 (pubmed: 25516281) was used for analyzing differentially expressed genes between proliferating (P) and non-proliferating (NP) cells

```
  > counts <- readRDS(‘count.rds’) ## read raw counts
  > group <- group[group %in% c(′P′,′NP′)]
  > cm <- as.matrix(counts[,names(group)])
  > meta <- data.frame(sample.id = colnames(cm), group =group)
  > dds1 <- DESeq2::DESeqDataSetFromMatrix(round(cm,0), meta, design = ~group)
  > meta$group <- relevel(meta$group, ref = ′NoPro′)
  > dds1 <- DESeq2::DESeq(dds1)
  > res <- DESeq2::results(dds1, cooksCutoff = FALSE, independentFiltering = FALSE)
  > res <- as.data.frame(res)
  > res <- res1[order(res $padj, decreasing = FALSE), ]
  Visualization of differentially expressed genes
  > library(EnhancedVolcano)
  ># create custom key-value pairs for ′high′, ′low′, ′mid′ expression by fold-change
  > keyvalssize <- ifelse(
  >res$log2FoldChange <= -2 & res$padj <0.01, 3,
  > ifelse(res$log2FoldChange >= 2 & res$padj <0.01, 3,
  > 1))
  > EnhancedVolcano(res, lab = rownames(res), x = ′log2FoldChange′, y = ′padj′,
title=NULL,
  > subtitle=NULL, caption=NULL,
  > selectLab = rownames(res)[which(names(keyvals) %in% c(′high′, ′low′))],
  > xlab = bquote(~Log[2]~ ′fold change′),
  > ylab = bquote(~Log[10]~ ′padj′),
  > pCutoff = 0.001, > FCcutoff = 2,
  > pointSize = keyvalssize, #3.5,
  > labSize = 4.5,
  > #shape = c(6, 4, 2, 11),
  > colCustom = keyvals,
  > colAlpha = 1,
  > legendPosition = ′none′,
  > legendLabSize = 15,
  > legendIconSize = 5.0,
  > drawConnectors = TRUE,
  > widthConnectors =1.0,
  > colConnectors = ′black′,
  > arrowheads = FALSE,
  > gridlines.major = TRUE,
  > gridlines.minor = FALSE,
  > border = ′partial′,
  > borderWidth = 1.5,
  > borderColour = ′black′)
  Clustering of cell cycle signature score
```

To assess cell states in different cell subsets and conditions, we used a gene set signature score to measure the relative difference of cell cycle states. The signature scores were calculated as average expression values of the genes in a given set. The signature gene list was downloaded from Whitfield et al (Identification of Genes Periodically Expressed in the Human Cell Cycle and Their Expression in Tumors. Mol Biol Cell 13, 1977-2000 (2002)). Hierarchical clustering of the cell cycle signature score was used to group AML, cells.

> df <- read.csv(′ CellCycleGeneList.txt′,sep=′\t′,header=T)
> allscore=NULL
> gl <- list( ) #gene list
> for (i in c(″M/G1″ , ″G2″ , ″G2/M″ , ″G1/S″ , ″S phase″ )){
> tmp1 <- df[df$PHASE == i,]
> gs <- apply(tmp1,1,function(x) strsplit(x[′NAME′],′ ′)[[1]][1])
> gs <- gs[gs !=″]
> Gsano <- readRDS(′gene.ano.rds′) # human and mouse homolog genes
> index 12 <- (gs %in% gsano$Gene_Name)
> index12 <- match(gs[index12],gsano$Gene_Name)
> gs2 <- Toch(gsano[index12,′mouse_homolog_gene′])
> gl[[i]]=gs2
> allscore <- cbind(allscore,rowMeans(as.matrix(exp[gs2,])))
> }
> Annot <- data.frame(′cluster′=tr3,′fraction′=dtype2[names(tr3)],row.names=names(tr3))
> colnames(allscore) <- c(″M/G1″ , ″G2″ , ″G2/M″ , ″G1/S″ , ″S phase″ )
> a <- pheatmap::pheatmap(allscore,annotation_row = annot)
> cluster <- cutree(a$tree_row,k=3)

What is claimed is:

1. A method for image-guided cell isolation from a region of interest of a subject, the method comprising the steps of:
imaging the subject using optical microscopy to identify the region of interest in a target anatomy;
inserting a micropipette into the region of interest under guidance of the optical microscopy;
aspirating at least one cell of a target population of cells in the region of interest under guidance of the optical microscopy; and
analyzing the at least one cell aspirated from the region of interest.

2. The method of claim 1, wherein imaging the subject using optical microscopy further comprises a one-photon imaging mode or a multi-photon imaging mode.

3. The method of claim 1, wherein the target population of cells further comprises a label configured to be identified using the optical microscopy.

4. The method of claim 3, wherein the label further comprises a fluorescently tagged molecule that is expressed by or selectively binds to the target population of cells.

5. The method of claim 1, wherein the target anatomy comprises a bone.

6. The method of claim 5, wherein the region of interest comprises a marrow cavity of the bone.

7. The method of claim 6, further comprising:
creating an opening in the bone configured to receive the micropipette.

8. The method of claim 7, wherein creating the opening further comprises:
creating the opening by directing an ablation laser beam operating in an ablation modality.

9. The method of claim 1, wherein the micropipette includes a coating comprising a fluorescent molecule.

10. The method of claim 1, wherein analyzing the target population of cells comprises single-cell RNA sequencing.

11. The method of claim 1, wherein analyzing the target population of cells comprises multi-omic analysis, cell transplantation, or colony formation assays.

12. The method of claim 1, further comprising dissecting the target anatomy from the subject before inserting the micropipette into the region of interest.

13. A method for image-guided tissue ablation from a region of interest of a subject, the method comprising the steps of:
imaging the subject using optical microscopy to identify a tissue in a region of interest of a target anatomy;
ablating the tissue in the region of interest using an ablation laser under guidance of the optical microscopy; and
confirming a spatial location of the ablated tissue using the optical microscopy.

14. The method of claim 13, wherein imaging the subject using optical microscopy further comprises a one-photon imaging mode or a multi-photon imaging mode.

15. The method of claim 13, wherein the tissue ablation is performed to provide access to a target population of cells in the tissue, and wherein the target cell population comprises a label configured to be identified using the optical microscopy.

16. The method of claim 15, wherein the label further comprises a fluorescently tagged molecule that is expressed by or selectively binds to the target population of cells.

17. The method of claim 13, wherein the target anatomy comprises a bone.

18. The method of claim 17, wherein the region of interest comprises a marrow cavity of the bone.

19. The method of claim 18, further comprising:
creating an opening in the bone.

20. The method of claim 19, wherein creating the opening further comprises:
creating the opening by directing an ablation laser operating in an ablation modality.

21. The method of claim 13, further comprising dissecting the target anatomy from the subject before ablating the tissue.

22. A system for image-guided cell isolation from a region of interest of a subject, the system comprising:
an optical microscope;
a micropipette assembly; and
a processor in communication with the optical microscope and the micropipette assembly, the processor configured to:
image the subject using the optical microscope to identify the region of interest in a target anatomy,
identify from one or more images generated of the subject at least one cell of the target population of cells in the region of interest; and
control the micropipette assembly to aspirate at least one cell of a target population of cells in the region of interest under guidance of the optical microscope.

23. The system of claim 22, further comprising an ablation laser, wherein the processor is further configured to control the ablation laser to transmit an ablation beam towards the target anatomy.

24. The system of claim 23, wherein the ablation laser is integrated with the optical microscope.

25. The system of claim 23, wherein the ablation laser emits the ablation beam at a repetition frequency in a range of 1 kHz to 10 MHz.

26. The system of claim 23, wherein the ablation laser emits the ablation beam at a pulse energy in a range of about 5 nJ to 1 μJ.

27. The system of claim 23, wherein the ablation laser emits the ablation beam at a pulse duration of less than 1 ps.

28. The system of claim 22, further comprising a flushing system, wherein the processor is further configured to control the flushing system to deliver and remove a flushing fluid across the target anatomy.

29. The system of claim 22, further comprising a microcontroller either in communication with the processor or operated independently, wherein the microcontroller is configured to move the micropipette under the guidance of the optical microscope.

30. The system of claim 29, further comprising a sliding stage either in communication with the processor or operated independently, wherein the sliding stage is configured to translate in a coordinate frame of reference relative to a sample stage.

31. The system of claim 22, wherein the optical microscope comprises one-photon or multi-photon imaging modes.
